(19) United States
(12) Patent Application Publication
(10) Pub. No.: US 2023/0407412 A1
(43) Pub. Date: Dec. 21, 2023

(54) MULTIPLEX DETECTION OF BACTERIAL RESPIRATORY PATHOGENS

(71) Applicant: Becton, Dickinson and Company, Franklin Lakes, NJ (US)

(72) Inventors: Qiufeng Zhang, Suzhou (CN); Manliang FU, Suzhou (CN); Chuanhui Zhang, Suzhou (CN)

(21) Appl. No.: 18/251,528

(22) PCT Filed: Nov. 4, 2021
(86) PCT No.: PCT/CN2021/128667

§ 371 Date: May 2, 2023

(30) Foreign Application Priority Data
Nov. 5, 2020 (WO) PCT/CN2020/126728

(51) Int. Cl. C12Q 1/689; C12Q 1/686

(52) U.S. Cl. CPC C12Q 1/689; C12Q 1/686; C12Q 2600/16

(57) ABSTRACT

Methods and compositions for detection of common bacterial pathogens causing respiratory infections are disclosed herein. In some embodiments, the presence or absence of Streptococcus pneumoniae, Haemophilus influenzae, Staphylococcus aureus, Moraxella (Branhamella) catarrhalis, Neisseria meningitides, and/or Klebsiella pneumoniae in a sample is determined using multiplex nucleic acid-based testing methods.

### RELATED APPLICATIONS

This application claims the benefit of PCT Application Serial No. PCT/CN2020/126728, filed Nov. 5, 2020, the content of this related application is incorporated herein by reference in its entirety.

### REFERENCE TO SEQUENCE LISTING

The present application is being filed along with a Sequence Listing in electronic format. The Sequence Listing is provided as a file entitled 68EB-298736-WO2, created Nov. 2, 2021, which is 28 kb in size. The information in the electronic format of the Sequence Listing is incorporated herein by reference in its entirety.

### BACKGROUND

### Field

The present disclosure relates to methods and compositions for the detection and of bacteria, such as Streptococcus pneumoniae, Haemophilus influenzae, Staphylococcus aureus, Moraxella (Branhamella) catarrhalis, Neisseria meningitides, and Klebsiella pneumoniae, in samples. More specifically, the present disclosure relates to the detection of one or more of Streptococcus pneumoniae, Haemophilus influenzae, Staphylococcus aureus, Moraxella (Branhamella) catarrhalis, Neisseria meningitides, and Klebsiella pneumoniae in samples, such as blood samples or respiratory samples (or cultures thereof), by nucleic acid-based testing methods.

### Description of the Related Art

Streptococcus pneumoniae, Haemophilus influenzae, Staphylococcus aureus, Moraxella (Branhamella) catarrhalis, Neisseria meningitides, and Klebsiella pneumoniae are six common pathogens associated with respiratory tract infections. Acute bacterial respiratory tract infections are among the most common human ailments. Symptoms range in severity from mild upper respiratory tract infections (URTI) to serious lower respiratory tract infections (LRTI), many of which are associated with significant morbidity and mortality particularly in children, the elderly and those who are immunocompromised or have underlying comorbidities such as congenital heart defect or chronic respiratory disease. Additionally, Streptococcus pneumoniae, Neisseria meningitides, and Haemophilus influenzae are the major pathogens frequently associated with bacterial meningitis worldwide with more than 1.2 million cases each year. K. pneumoniae has been identified as a major nosocomial pathogen, which can cause pneumonia, bronchitis, urinary tract and wound infections. Moreover, antibiotic-resistant K. pneumoniae emerged in recent years has become a serious problem in clinics. M. catarrhalis is one of the major bacterial pathogens causing otitis media in children. It also causes sinusitis, laryngitis, tracheitis, pneumonia, and other respiratory diseases in children and adults. Also, the mortality rate of S. aureus infections and the complications thereof is as high as 20%, methicillin-resistant Staphylococcus aureus (MRSA) has become one of the pathogens with the highest hospital infection rate, such as ICU infection, post-operation infection. Timely identification of all these six pathogens from infected patients in fever is important for antimicrobial stewardship and disease control.

Current standard diagnostic methods for respiratory bacteria are culture-based and typically take 24-72 hours. Culture also has low sensitivity, for example a positive microbiological diagnosis may only be made in approximately 30% of patients with community-acquired pneumonia. Additionally, errors in phenotypic identification may occur, even when matrix-assisted laser desorption/ionization time-of-flight mass spectrometry (MALDI-TOF MS) technology is used. Indeed, Cunningham et al. recently reported the misidentification of N. polysaccharea strains as N. meningitidis using MALDI-TOF MS with high identification scores ranging from 2.068 to 2.241. Such misidentification is expected because these two species are closely related, as demonstrated by their high percentage of 16S rRNA identity (99.3%).

As a wide range of pathogens can cause respiratory tract infection, it is common to request a mixture of different diagnostic tests, including culture, antigen testing, and serology, on a number of different specimen types. In contrast, the use of PCR, for example multiplex PCR assays, can enable screening for a large number of respiratory pathogens in a single respiratory specimen within the working day. Besides, the PCR method has many other advantages: it is highly sensitive and specific, shorter time to result, less labor-intensive and the ability to identify pathogens that are slow-growing or difficult to culture. Accordingly, there is a need for developing more efficient and faster methods for detecting bacterial respiratory pathogens, for example a method allowing detecting of two or more of Streptococcus pneumoniae, Haemophilus influenzae, Staphylococcus aureus, Moraxella (Branhamella) catarrhalis, Neisseria meningitides, and Klebsiella pneumoniae in a single assay, in order to effectively deliver proper treatments to patients. In particular, there is a need for multiplex real-time PCR methods capable of simultaneously detecting the above-mentioned respiratory pathogens with more specific and inclusive gene markers.

### SUMMARY

Disclosed herein include methods of detecting S. pneumoniae and N. meningitidis in a sample. In some embodiments, the method comprises: contacting said sample with a plurality of pairs of primers, wherein the plurality of pairs of primers comprises: at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity to any one of the sequences of SEQ ID NOs: 1-10; at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity to any one of the sequences of SEQ ID NOs: 16-25; at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity to any one of the sequences of SEQ ID NOs: 31-39; and at least one pair of primers capable of hybridizing to the crtA gene of N. meningitidis, wherein each primer in said at least one pair of primers comprises any one of the sequences of SEQ ID NOs: 45-54, or a sequence that exhibits at least about 85% identity to any one of the sequences of SEQ ID NOs: 45-54. The method can comprise: generating amplicons of the lytA gene sequence of S. pneumoniae, amplicons of the cpsA gene sequence of S. pneumoniae, amplicons of the sodC gene sequence of N. meningitidis, amplicons of the crtA gene sequence of N. meningitidis from said sample, or any combination thereof, if said sample comprises one or both of S. pneumoniae and N. meningitidis. The method can comprise: determining the presence or amount of one or more amplicons as an indication of the presence of one or both of S. pneumoniae and N. meningitidis in said sample. The method can comprise: contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132, and generating amplicons of the IAC from said sample; and determining the presence or amount of the amplicons of the IAC as an indication of the performance of the assay with the sample. In some embodiments, the sample is contacted with a composition comprising the plurality of pairs of primers and the at least one pair of control primers capable of hybridizing to the IAC.

In some embodiments, the sample is a biological sample or an environmental sample. In some embodiments, the environmental sample is obtained from a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. In some embodiments, the biological sample is obtained from a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof. In some embodiments, the biological sample comprises a blood sample, a respiratory sample, and/or cultures thereof.

In some embodiments, the plurality of pairs of primers comprises a first primer comprising the sequence of SEQ ID NOs: 1, 3, 5, 7, or 9, a second primer comprising the sequence of SEQ ID NOs: 2, 4, 6, 8, or 10, a third primer comprising the sequence of SEQ ID NOs: 16, 18, 20, 22, or 24, a fourth primer comprising the sequence of SEQ ID NOs: 17, 19, 21, 23, or 25, a fifth primer comprising the sequence of SEQ ID NOs: 31, 33, 35, 36, or 38, a sixth primer comprising the sequence of SEQ ID NOs: 32, 34, 37, or 39, a seventh primer comprising the sequence of SEQ ID NOs: 45, 47, 49, 51, or 53, and an eighth primer comprising the sequence of SEQ ID NOs: 46, 48, 50, 52, or 54. In some embodiments, the plurality of pairs of primers comprises an ninth primer comprising the sequence of SEQ ID NOs: 60, 62, 64, 66, 68, or 131, and a tenth primer comprising the sequence of SEQ ID NOs: 61, 63, 65, 67, 69, 130, or 132.

In some embodiments, the pair of primers capable of hybridizing to the lytA gene of S. pneumoniae is SEQ ID NOs: 1 and 2, SEQ ID NOs: 3 and 4, SEQ ID NOs: 5 and 6, SEQ ID NOs: 7 and 8, or SEQ ID NOs: 9 and 10; the pair of primers capable of hybridizing to the cpsA gene of S. pneumoniae is SEQ ID NOs: 16 and 17, SEQ ID NOs: 18 and 19, SEQ ID NOs: 20 and 21, SEQ ID NOs: 22 and 23, or SEQ ID NOs: 24 and 25; the pair of primers capable of hybridizing to the sodC gene of N. meningitidis is SEQ ID NOs: 31 and 32, SEQ ID NOs: 33 and 34, SEQ ID NOs: 35 and 32, SEQ ID NOs: 36 and 37, or SEQ ID NOs: 38 and 39; and the pair of primers capable of hybridizing to the crtA gene of N. meningitidisis is SEQ ID NOs: 45 and 46, SEQ ID NOs: 47 and 48, SEQ ID NOs: 49 and 50, SEQ ID NOs: 51 and 52, or SEQ ID NOs: 53 and 54. In some embodiments, the pair of control primers capable of hybridizing to the IAC is SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 61 and 130, or SEQ ID NOs: 131 and 132.

In some embodiments, said amplification is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA). In some embodiments, said PCR is real-time PCR. In some embodiments, said PCR is quantitative real-time PCR (QRT-PCR). In some embodiments, each primer comprises exogenous nucleotide sequence.

In some embodiments, determining the presence or amount of one or more amplicons comprises contacting the amplicons with a plurality of oligonucleotide probes, wherein each of the plurality of oligonucleotide probes comprises a sequence selected from the group consisting of SEQ ID NOs: 11-15, 26-30, 40-44, 55-59, 70-74, and 133-134, or a sequence that exhibits at least about 85% identity to a sequence selected from the group consisting of SEQ ID NOs: 11-15, 26-30, 40-44, 55-59, 70-74, and 133-134. In some embodiments, each of the plurality of oligonucleotide probes comprises a sequence selected from the group consisting of SEQ ID NOs: 11-15, 26-30, 40-44, 55-59, 70-74, and 133-134. In some embodiments, each of the plurality of oligonucleotide probes consists of a sequence selected from the group consisting of SEQ ID NOs: 11-15, 26-30, 40-44, 55-59, 70-74, and 133-134. In some embodiments, each probe is flanked by complementary sequences at the 5′ end and 3′ end. In some embodiments, one of the complementary sequences comprises a fluorescence emitter moiety and the other complementary sequence comprises a fluorescence quencher moiety. In some embodiments, at least one of the plurality of oligonucleotide probes comprises a fluorescence emitter moiety and a fluorescence quencher moiety.

Disclosed herein include compositions for detecting S. pneumoniae and N. meningitidis in a sample. In some embodiments, the composition comprises: at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity to any one of the sequences of SEQ ID NOs: 1-10; at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity to any one of the sequences of SEQ ID NOs: 16-25; at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity to any one of the sequences of SEQ ID NOs: 31-39; and at least one pair of primers capable of hybridizing to the crtA gene of N. meningitidis, wherein each primer in said at least one pair of primers comprises any one of the sequences of SEQ ID NOs: 45-54, or sequence that exhibits at least about 85% identity to any one of the sequences of SEQ ID NOs: 45-54. The composition can comprise: at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132.

In some embodiments, the at least one pair of primers capable of hybridizing to the lytA gene of S. pneumoniae comprises a primer comprising the sequence of SEQ ID NOs: 1, 3, 5, 7, or 9 and a primer comprising the sequence of SEQ ID NOs: 2, 4, 6, 8, or 10; the at least one pair of primers capable of hybridizing to the cpsA gene of S. pneumoniae comprises a primer comprising the sequence of SEQ ID NOs: 16, 18, 20, 22, or 24 and a primer comprising the sequence of SEQ ID NOs: 17, 19, 21, 23, or 25; the at least one pair of primers capable of hybridizing to the sodC gene of N. meningitidis comprises a primer comprising the sequence of SEQ ID NOs: 31, 33, 35, 36, or 38 and a primer comprising the sequence of SEQ ID NOs: 32, 34, 37, or 39; and the at least one pair of primers capable of hybridizing to the crtA gene of N. meningitidis comprises a primer comprising the sequence of SEQ ID NOs: 45, 47, 49, 51, or 53 and a primer comprising the sequence of SEQ ID NOs: 46, 48, 50, 52, or 54. In some embodiments, the at least one pair of control primers capable of hybridizing to the IAC comprises a primer comprising the sequence of SEQ ID NOs: 60, 62, 64, 66, 68, or 131 and a primer comprising the sequence of SEQ ID NOs: 61, 63, 65, 67, 69, 130, or 132.

The composition can comprise: a plurality of oligonucleotide probes, wherein each of the plurality of oligonucleotide probes comprises a sequence selected from the group consisting of SEQ ID NOs: 11-15, 26-30, 40-44, 55-59, 70-74, and 133-134, or a sequence that exhibits at least about 85% identity to a sequence selected from the group consisting of SEQ ID NOs: 11-15, 26-30, 40-44, 55-59, 70-74, and 133-134. In some embodiments, each of the plurality of oligonucleotide probes comprises a sequence selected from the group consisting of SEQ ID NOs: 11-15, 26-30, 40-44, 55-59, 70-74, and 133-134. In some embodiments, each of the plurality of oligonucleotide probes consists of a sequence selected from the group consisting of SEQ ID NOs: 11-15, 26-30, 40-44, 55-59, 70-74, and 133-134. In some embodiments, at least one of the plurality of probes comprises a fluorescence emitter moiety and a fluorescence quencher moiety.

Disclosed herein include methods of detecting S. aureus, H. influenzae, M. catarrhalis, and K. pneumoniae in a sample. In some embodiments, the method comprises: contacting said sample with a plurality of pairs of primers, wherein the plurality of pairs of primers comprises: at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity to any one of the sequences of SEQ ID NOs: 75-83; at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity to any one of the sequences of SEQ ID NOs: 88-95; at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity to any one of the sequences of SEQ ID NOs: 101-110; and at least one pair of primers capable of hybridizing to the gltA gene of K. pneumoniae, wherein each primer in said at least one pair of primers comprises any one of the sequences of SEQ ID NOs: 116-124, or a sequence that exhibits at least about 85% identity to any one of the sequences of SEQ ID NOs: 116-124. The method can comprise: generating amplicons of the nuc gene sequence of S. aureus, amplicons of the fucK gene sequence of H. influenzae, amplicons of the gene sequence of the copB gene sequence of M. catarrhalis, amplicons of the gltA gene sequence of K. pneumoniae from said sample, or any combination thereof, if said sample comprises one or more of S. aureus, H. influenzae, M. catarrhalis, and K. pneumoniae. The method can comprise: determining the presence or amount of one or more amplicons as an indication of the presence of one or more of S. aureus, H influenzae, M. catarrhalis, and K. pneumoniae in said sample. The method can comprise: contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132, and generating amplicons of the IAC from said sample; and determining the presence or amount the amplicons of the IAC as an indication of the performance of the assay with the sample. In some embodiments, the sample is contacted with a composition comprising the plurality of pairs of primers and the at least one pair of control primers capable of hybridizing to the IAC.

In some embodiments, the sample is a biological sample or an environmental sample. In some embodiments, the environmental sample is obtained from a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. In some embodiments, the biological sample is obtained from a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof. In some embodiments, the biological sample comprises a blood sample, a respiratory sample, and/or cultures thereof.

In some embodiments, the plurality of pairs of primers comprises a first primer comprising the sequence of SEQ ID NOs: 75, 77, 79, 81, or 83, a second primer comprising the sequence of SEQ ID NOs: 76, 78, 80, or 82, a third primer comprising the sequence of SEQ ID NOs: 88, 90, 92, 93, or 94, a fourth primer comprising the sequence of SEQ ID NOs: 89, 91, or 95, a fifth primer comprising the sequence of SEQ ID NOs: 101, 103, 105, 107, or 109, a sixth primer comprising the sequence of SEQ ID NOs: 102, 104, 106, 108, or 110, a seventh primer comprising the sequence of SEQ ID NOs: 116, 118, 120, or 122, and an eighth primer comprising the sequence of SEQ ID NOs: 117, 119, 121, 123, or 124. In some embodiments, the plurality of pairs of primers comprises an ninth primer comprising the sequence of SEQ ID NOs: 60, 62, 64, 66, 68, or 131, and a tenth primer comprising the sequence of SEQ ID NOs: 61, 63, 65, 67, 69, 130, or 132.

In some embodiments, the pair of primers capable of hybridizing to the nuc gene of S. aureus is SEQ ID NOs: 75 and 76, SEQ ID NOs: 77 and 78, SEQ ID NOs: 79 and 80, SEQ ID NOs: 81 and 82, or SEQ ID NOs: 83 and 78; the pair of primers capable of hybridizing to the fucK gene of H. influenzae is SEQ ID NOs: 88 and 89, SEQ ID NOs: 90 and 91, SEQ ID NOs: 92 and 89, SEQ ID NOs: 93 and 89, or SEQ ID NOs: 94 and 95; the pair of primers capable of hybridizing to the copB gene of M. catarrhalis is SEQ ID NOs: 101 and 102, SEQ ID NOs: 103 and 104, SEQ ID NOs: 105 and 106, SEQ ID NOs: 107 and 108, or SEQ ID NOs: 109 and 110; and the pair of primers capable of hybridizing to the gltA gene of K. pneumoniae is SEQ ID NOs: 116 and 117, SEQ ID NOs: 118 and 119, SEQ ID NOs: 120 and 121, SEQ ID NOs: 122 and 123, or SEQ ID NOs: 116 and 124. In some embodiments, the pair of control primers capable of hybridizing to the IAC is SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 61 and 130, or SEQ ID NOs: 131 and 132.

In some embodiments, said amplification is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA). In some embodiments, said PCR is real-time PCR. In some embodiments, said PCR is quantitative real-time PCR (QRT-PCR). In some embodiments, each primer comprises exogenous nucleotide sequence.

In some embodiments, determining the presence or amount of one or more amplicons comprises contacting the amplicons with a plurality of oligonucleotide probes, wherein each of the plurality of oligonucleotide probes comprises a sequence selected from the group consisting of SEQ ID NOs: 84-87, 96-100, 111-115, 125-129, 70-74, and 133-134, or a sequence that exhibits at least about 85% identity to a sequence selected from the group consisting of SEQ ID NOs: 84-87, 96-100, 111-115, 125-129, 70-74, and 133-134. In some embodiments, each of the plurality of oligonucleotide probes comprises a sequence selected from the group consisting of SEQ ID NOs: 84-87, 96-100, 111-115, 125-129, 70-74, and 133-134. In some embodiments, each of the plurality of oligonucleotide probes consists of a sequence selected from the group consisting of SEQ ID NOs: 84-87, 96-100, 111-115, 125-129, 70-74, and 133-134. In some embodiments, each probe is flanked by complementary sequences at the 5′ end and 3′ end. In some embodiments, one of the complementary sequences comprises a fluorescence emitter moiety and the other complementary sequence comprises a fluorescence quencher moiety. In some embodiments, at least one of the plurality of oligonucleotide probes comprises a fluorescence emitter moiety and a fluorescence quencher moiety.

Disclosed herein include compositions for detecting S. aureus, H. influenzae, M. catarrhalis, and K. pneumoniae in a sample. In some embodiments, the composition comprises: at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity to any one of the sequences of SEQ ID NOs: 75-83; at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity to any one of the sequences of SEQ ID NOs: 88-95; at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity to any one of the sequences of SEQ ID NOs: 101-110; and at least one pair of primers capable of hybridizing to the gltA gene of K. pneumoniae, wherein each primer in said at least one pair of primers comprises any one of the sequences of SEQ ID NOs: 116-124, or sequence that exhibits at least about 85% identity to any one of the sequences of SEQ ID NOs: 116-124. The composition can comprise: at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132.

In some embodiments, the at least one pair of primers capable of hybridizing to the nuc gene of S. aureus comprises a primer comprising the sequence of SEQ ID NOs: 75, 77, 79, 81, or 83 and a primer comprising the sequence of SEQ ID NOs: 76, 78, 80, or 82; the at least one pair of primers capable of hybridizing to the fucK gene of H. influenzae comprises a primer comprising the sequence of SEQ ID NOs: 88, 90, 92, 93, or 94 and a primer comprising the sequence of SEQ ID NOs: 89, 91, or 95; the at least one pair of primers capable of hybridizing to the copB gene of M. catarrhalis comprises a primer comprising the sequence of SEQ ID NOs: 101, 103, 105, 107, or 109 and a primer comprising the sequence of SEQ ID NOs: 102, 104, 106, 108, or 110; and the at least one pair of primers capable of hybridizing to the gltA gene of K. pneumoniae comprises a primer comprising the sequence of SEQ ID NOs: 116, 118, 120, or 122 and a primer comprising the sequence of SEQ ID NOs: 117, 119, 121, 123, or 124. In some embodiments, the at least one pair of control primers capable of hybridizing to the IAC comprises a primer comprising the sequence of SEQ ID NOs: 60, 62, 64, 66, 68, or 131 and a primer comprising the sequence of SEQ ID NOs: 61, 63, 65, 67, 69, 130, or 132.

The composition can comprise: a plurality of oligonucleotide probes, wherein each of the plurality of oligonucleotide probes comprises a sequence selected from the group consisting of SEQ ID NOs: 84-87, 96-100, 111-115, 125-129, 70-74, and 133-134, or a sequence that exhibits at least about 85% identity to a sequence selected from the group consisting of SEQ ID NOs: 84-87, 96-100, 111-115, 125-129, 70-74, and 133-134. In some embodiments, each of the plurality of oligonucleotide probes comprises a sequence selected from the group consisting of SEQ ID NOs: 84-87, 96-100, 111-115, 125-129, 70-74, and 133-134. In some embodiments, each of the plurality of oligonucleotide probes consists of a sequence selected from the group consisting of SEQ ID NOs: 84-87, 96-100, 111-115, 125-129, 70-74, and 133-134. In some embodiments, at least one of the plurality of probes comprises a fluorescence emitter moiety and a fluorescence quencher moiety.

Disclosed herein include probes or primers up to about 100 nucleotides in length which is capable of hybridizing to the lytA gene of S. pneumoniae. In some embodiments, the probe or primer comprises: a sequence selected from the group consisting of SEQ ID NOs: 1-15, or sequence that exhibits at least about 85% identity to a sequence selected from the group consisting of SEQ ID NOs: 1-15. In some embodiments, said probe or primer consists of a sequence selected from the group consisting of SEQ ID NOs: 1-15, or sequence that exhibits at least about 85% identity to a sequence selected from the group consisting of SEQ ID NOs: 1-15. In some embodiments, said probe or primer comprises a sequence selected from the group consisting of SEQ ID NOs: 1-15. In some embodiments, said probe or primer consists of a sequence selected from the group consisting of SEQ ID NOs: 1-15.

Disclosed herein include probes or primers up to about 100 nucleotides in length which is capable of hybridizing to the cpsA gene of S. pneumoniae. In some embodiments, the probe or primer comprises: a sequence selected from the group consisting of SEQ ID NOs: 16-30, or sequence that exhibits at least about 85% identity to a sequence selected from the group consisting of SEQ ID NOs: 16-30. In some embodiments, said probe or primer consists of a sequence selected from the group consisting of SEQ ID NOs: 16-30, or sequence that exhibits at least about 85% identity to a sequence selected from the group consisting of SEQ ID NOs: 16-30. In some embodiments, said probe or primer comprises a sequence selected from the group consisting of SEQ ID NOs: 16-30. In some embodiments, said probe or primer consists of a sequence selected from the group consisting of SEQ ID NOs: 16-30.

Disclosed herein include probes or primers up to about 100 nucleotides in length which is capable of hybridizing to the sodC gene of N. meningitidis. In some embodiments, the probe or primer comprises: a sequence selected from the group consisting of SEQ ID NOs: 31-44, or sequence that exhibits at least about 85% identity to a sequence selected from the group consisting of SEQ ID NOs: 31-44. In some embodiments, said probe or primer consists of a sequence selected from the group consisting of SEQ ID NOs: 31-44, or sequence that exhibits at least about 85% identity to a sequence selected from the group consisting of SEQ ID NOs: 31-44. In some embodiments, said probe or primer comprises a sequence selected from the group consisting of SEQ ID NOs: 31-44. In some embodiments, said probe or primer consists of a sequence selected from the group consisting of SEQ ID NOs: 31-44.

Disclosed herein include probes or primers up to about 100 nucleotides in length which is capable of hybridizing to the crtA gene of N. meningitidis. In some embodiments, the probe or primer comprises: a sequence selected from the group consisting of SEQ ID NOs: 45-59, or sequence that exhibits at least about 85% identity to a sequence selected from the group consisting of SEQ ID NOs: 45-59. In some embodiments, said probe or primer consists of a sequence selected from the group consisting of SEQ ID NOs: 45-59, or sequence that exhibits at least about 85% identity to a sequence selected from the group consisting of SEQ ID NOs: 45-59. In some embodiments, said probe or primer comprises a sequence selected from the group consisting of SEQ ID NOs: 45-59. In some embodiments, said probe or primer consists of a sequence selected from the group consisting of SEQ ID NOs: 45-59.

Disclosed herein include probes or primers up to about 100 nucleotides in length which is capable of hybridizing to an internal amplification control (IAC). In some embodiments, the probe or primer comprises: a sequence selected from the group consisting of SEQ ID NOs: 60-74 and 130-134, or sequence that exhibits at least about 85% identity to a sequence selected from the group consisting of SEQ ID NOs: 60-74 and 130-134. In some embodiments, said probe or primer consists of a sequence selected from the group consisting of SEQ ID NOs: 60-74 and 130-134, or sequence that exhibits at least about 85% identity to a sequence selected from the group consisting of SEQ ID NOs: 60-74 and 130-134. In some embodiments, said probe or primer comprises a sequence selected from the group consisting of SEQ ID NOs: 60-74 and 130-134. In some embodiments, said probe or primer consists of a sequence selected from the group consisting of SEQ ID NOs: 60-74 and 130-134.

Disclosed herein include probes or primers up to about 100 nucleotides in length which is capable of hybridizing to the nuc gene of S. aureus. In some embodiments, the probe or primer comprises: a sequence selected from the group consisting of SEQ ID NOs: 75-87, or sequence that exhibits at least about 85% identity to a sequence selected from the group consisting of SEQ ID NOs: 75-87. In some embodiments, said probe or primer consists of a sequence selected from the group consisting of SEQ ID NOs: 75-87, or sequence that exhibits at least about 85% identity to a sequence selected from the group consisting of SEQ ID NOs: 75-87. In some embodiments, said probe or primer comprises a sequence selected from the group consisting of SEQ ID NOs: 75-87. In some embodiments, said probe or primer consists of a sequence selected from the group consisting of SEQ ID NOs: 75-87.

Disclosed herein include probes or primers up to about 100 nucleotides in length which is capable of hybridizing to the fucK gene of H. influenzae. In some embodiments, the probe or primer comprises: a sequence selected from the group consisting of SEQ ID NOs: 88-100, or sequence that exhibits at least about 85% identity to a sequence selected from the group consisting of SEQ ID NOs: 88-100. In some embodiments, said probe or primer consists of a sequence selected from the group consisting of SEQ ID NOs: 88-100, or sequence that exhibits at least about 85% identity to a sequence selected from the group consisting of SEQ ID NOs: 88-100. In some embodiments, said probe or primer comprises a sequence selected from the group consisting of SEQ ID NOs: 88-100. In some embodiments, said probe or primer consists of a sequence selected from the group consisting of SEQ ID NOs: 88-100.

Disclosed herein include probes or primers up to about 100 nucleotides in length which is capable of hybridizing to the copB gene of M. catarrhalis. In some embodiments, the probe or primer comprises: a sequence selected from the group consisting of SEQ ID NOs: 101-115, or sequence that exhibits at least about 85% identity to a sequence selected from the group consisting of SEQ ID NOs: 101-115. In some embodiments, said probe or primer consists of a sequence selected from the group consisting of SEQ ID NOs: 101-115, or sequence that exhibits at least about 85% identity to a sequence selected from the group consisting of SEQ ID NOs: 101-115. In some embodiments, said probe or primer comprises a sequence selected from the group consisting of SEQ ID NOs: 101-115. In some embodiments, said probe or primer consists of a sequence selected from the group consisting of SEQ ID NOs: 101-115.

Disclosed herein include probes or primers up to about 100 nucleotides in length which is capable of hybridizing to the gltA gene of K. pneumoniae. In some embodiments, the probe or primer comprises: a sequence selected from the group consisting of SEQ ID NOs: 116-129, or sequence that exhibits at least about 85% identity to a sequence selected from the group consisting of SEQ ID NOs: 116-129. In some embodiments, said probe or primer consists of a sequence selected from the group consisting of SEQ ID NOs: 116-129, or sequence that exhibits at least about 85% identity to a sequence selected from the group consisting of SEQ ID NOs: 116-129. In some embodiments, said probe or primer comprises a sequence selected from the group consisting of SEQ ID NOs: 116-129. In some embodiments, said probe or primer consists of a sequence selected from the group consisting of SEQ ID NOs: 116-129.

Disclosed herein include compositions. In some embodiments, the composition comprises two or more of the oligonucleotide probes and/or primers disclosed herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

As a wide range of pathogens can cause respiratory tract infection, it is common to request a mixture of different diagnostic tests, including culture, antigen testing, and serology, on a number of different specimen types. In contrast, the use of PCR especially multiplex PCR assays can enable screening for a large number of respiratory pathogens in a single respiratory specimen within the working day. Besides, the PCR method has many other advantages: it is highly sensitive and specific, shorter time to result, less labor-intensive and the ability to identify pathogens that are slow-growing or difficult to culture.

Prior arts adapted PCR method for some common respiratory bacteria have been described, but seldom assay covers the wide range of Gram-positive and Gram-negative pathogens required for respiratory tract infection diagnosis. Ouattara, M et al. (Diagnostic microbiology and infectious disease 93.3 2019: 188-190.) described a Tagman probe real-time PCR method, which can only detect S. pneumoniae, N. meningitidis, and H. influenzae. Hasan, M. R et al. (Journal of virological methods 265 2019: 42-48) described a set of singleplex real-time PCR for the detection of S. pneumoniae, Bordetella pertussis, Mycoplasma pneumoniae, Chlamydia pneumoniae and other respiratory viruses using two sets of 8-tube strips. Furthermore, some assays have a lack of sensitivity and/or specificity for organisms such as N. meningitides, S. pneumoniae, and H. influenzae, owing to the use of suboptimal gene targets. Several RT-PCR assays for N. meningitidis target the capsule transport gene, ctrA. However, over 16% of meningococcal carriage isolates lack ctrA, rendering this target gene ineffective at the identification of this sub-population of meningococcal isolates. The sodC gene may offer an alternative as it is also specific for N. meningitidis. However, sodC PCR was suggested to be less sensitive than that of ctrA. Target genes for the detection of S. pneumoniae include the pneumolysin (ply), autolysin (lytA), and 16S rRNA gene. However, false-positive results with ply-based PCR have been reported when applied to upper respiratory tract specimens, because the respiratory flora (e.g., Streptococcus mitis group and Streptococcus oralis) sometimes contain a ply gene. In addition, S. pneumoniae shares over 99% 16S rRNA gene identity with S. mitis and S. oralis. The commonly used protein D-encoding gene hpd target for H. influenzae was known for the cross-reaction with Haemophilus aphrophilus. For the detection of K. pneumoniae, the inclusivity is a key influencing factor considering the existence of three distinct phylogroups called KpI, KpII, and KpIII, with KpI being the most prevalent. KpIII and KpII respectively were identified, which corresponded with two new bacterial species, K. variicola, and K. quasipneumoniae. These bacterial species, like K. pneumoniae, are commonly multidrug-resistant and can harbor cephalosporinase or carbapenemase genes. To achieve better care delivery and clinical utility, the target gene should conserve among all these three pathogens. However, the commonly used marker gapA gene can only be detected in KPI and KPIII.

Accordingly, there is a need for developing more efficient and faster methods for detecting respiratory pathogens, for example a method allowing detecting of two or more of Streptococcus pneumoniae, Haemophilus influenzae, Staphylococcus aureus, Moraxella (Branhamella) catarrhalis, Neisseria meningitides, and Klebsiella pneumoniae in a single assay, in order to effectively deliver proper treatments to patients. In particular, there is a need for multiplex real-time PCR methods capable of simultaneously detecting the above-mentioned respiratory pathogens with more specific and inclusive gene markers.

Provided herein are methods and compositions for the detection of bacterial respiratory pathogens. For example, primers and probes that can bind to specific genes of Streptococcus pneumoniae, Haemophilus influenzae, Staphylococcus aureus, Moraxella (Branhamella) catarrhalis, Neisseria meningitides, and Klebsiella pneumoniae are provided to determine the presence or absence of Streptococcus pneumoniae, Haemophilus influenzae, Staphylococcus aureus, Moraxella (Branhamella) catarrhalis, Neisseria meningitides, and Klebsiella pneumoniae in a sample, such as a biological sample. In some embodiments, multiplex nucleic acid amplification can be performed to allow the detection of S. pneumoniae and N. meningitidis in a single assay and/or detection of S. aureus, H. influenzae, M. catarrhalis, and K. pneumoniae in a single assay.

Disclosed herein include methods of detecting S. pneumoniae and N. meningitidis in a sample. In some embodiments, the method comprises: contacting said sample with a plurality of pairs of primers, wherein the plurality of pairs of primers comprises: at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity to any one of the sequences of SEQ ID NOs: 1-10; at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity to any one of the sequences of SEQ ID NOs: 16-25; at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity to any one of the sequences of SEQ ID NOs: 31-39; and at least one pair of primers capable of hybridizing to the crtA gene of N. meningitidis, wherein each primer in said at least one pair of primers comprises any one of the sequences of SEQ ID NOs: 45-54, or a sequence that exhibits at least about 85% identity to any one of the sequences of SEQ ID NOs: 45-54. The method can comprise: generating amplicons of the lytA gene sequence of S. pneumoniae, amplicons of the cpsA gene sequence of S. pneumoniae, amplicons of the sodC gene sequence of N. meningitidis, amplicons of the crtA gene sequence of N. meningitidis from said sample, or any combination thereof, if said sample comprises one or both of S. pneumoniae and N. meningitidis. The method can comprise: determining the presence or amount of one or more amplicons as an indication of the presence of one or both of S. pneumoniae and N. meningitidis in said sample. The method can comprise: contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132, and generating amplicons of the IAC from said sample; and determining the presence or amount of the amplicons of the IAC as an indication of the performance of the assay with the sample. In some embodiments, the sample is contacted with a composition comprising the plurality of pairs of primers and the at least one pair of control primers capable of hybridizing to the IAC.

Disclosed herein include compositions for detecting S. pneumoniae and N. meningitidis in a sample. In some embodiments, the composition comprises: at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity to any one of the sequences of SEQ ID NOs: 1-10; at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity to any one of the sequences of SEQ ID NOs: 16-25; at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity to any one of the sequences of SEQ ID NOs: 31-39; and at least one pair of primers capable of hybridizing to the crtA gene of N. meningitidis, wherein each primer in said at least one pair of primers comprises any one of the sequences of SEQ ID NOs: 45-54, or sequence that exhibits at least about 85% identity to any one of the sequences of SEQ ID NOs: 45-54. The composition can comprise: at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132. The composition can comprise: a plurality of oligonucleotide probes, wherein each of the plurality of oligonucleotide probes comprises a sequence selected from the group consisting of SEQ ID NOs: 11-15, 26-30, 40-44, 55-59, 70-74, and 133-134, or a sequence that exhibits at least about 85% identity to a sequence selected from the group consisting of SEQ ID NOs: 11-15, 26-30, 40-44, 55-59, 70-74, and 133-134.

Disclosed herein include methods of detecting S. aureus, H. influenzae, M. catarrhalis, and K. pneumoniae in a sample. In some embodiments, the method comprises: contacting said sample with a plurality of pairs of primers, wherein the plurality of pairs of primers comprises: at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity to any one of the sequences of SEQ ID NOs: 75-83; at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity to any one of the sequences of SEQ ID NOs: 88-95; at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity to any one of the sequences of SEQ ID NOs: 101-110; and at least one pair of primers capable of hybridizing to the gltA gene of K. pneumoniae, wherein each primer in said at least one pair of primers comprises any one of the sequences of SEQ ID NOs: 116-124, or a sequence that exhibits at least about 85% identity to any one of the sequences of SEQ ID NOs: 116-124. The method can comprise: generating amplicons of the nuc gene sequence of S. aureus, amplicons of the fucK gene sequence of H. influenzae, amplicons of the gene sequence of the copB gene sequence of M. catarrhalis, amplicons of the gltA gene sequence of K. pneumoniae from said sample, or any combination thereof, if said sample comprises one or more of S. aureus, H. influenzae, M. catarrhalis, and K. pneumoniae. The method can comprise: determining the presence or amount of one or more amplicons as an indication of the presence of one or more of S. aureus, H. influenzae, M. catarrhalis, and K. pneumoniae in said sample. The method can comprise: contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132, and generating amplicons of the IAC from said sample; and determining the presence or amount the amplicons of the IAC as an indication of the performance of the assay with the sample. In some embodiments, the sample is contacted with a composition comprising the plurality of pairs of primers and the at least one pair of control primers capable of hybridizing to the IAC.

Disclosed herein include compositions for detecting S. aureus, H. influenzae, M. catarrhalis, and K. pneumoniae in a sample. In some embodiments, the composition comprises: at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity to any one of the sequences of SEQ ID NOs: 75-83; at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity to any one of the sequences of SEQ ID NOs: 88-95; at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity to any one of the sequences of SEQ ID NOs: 101-110; and at least one pair of primers capable of hybridizing to the gltA gene of K. pneumoniae, wherein each primer in said at least one pair of primers comprises any one of the sequences of SEQ ID NOs: 116-124, or sequence that exhibits at least about 85% identity to any one of the sequences of SEQ ID NOs: 116-124. The composition can comprise: at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132. The composition can comprise: a plurality of oligonucleotide probes, wherein each of the plurality of oligonucleotide probes comprises a sequence selected from the group consisting of SEQ ID NOs: 84-87, 96-100, 111-115, 125-129, 70-74, and 133-134, or a sequence that exhibits at least about 85% identity to a sequence selected from the group consisting of SEQ ID NOs: 84-87, 96-100, 111-115, 125-129, 70-74, and 133-134.

Disclosed herein include probes or primers up to about 100 nucleotides in length which is capable of hybridizing to the lytA gene of S. pneumoniae. In some embodiments, the probe or primer comprises: a sequence selected from the group consisting of SEQ ID NOs: 1-15, or sequence that exhibits at least about 85% identity to a sequence selected from the group consisting of SEQ ID NOs: 1-15.

Disclosed herein include probes or primers up to about 100 nucleotides in length which is capable of hybridizing to the cpsA gene of S. pneumoniae. In some embodiments, the probe or primer comprises: a sequence selected from the group consisting of SEQ ID NOs: 16-30, or sequence that exhibits at least about 85% identity to a sequence selected from the group consisting of SEQ ID NOs: 16-30.

Disclosed herein include probes or primers up to about 100 nucleotides in length which is capable of hybridizing to the sodC gene of N. meningitidis. In some embodiments, the probe or primer comprises: a sequence selected from the group consisting of SEQ ID NOs: 31-44, or sequence that exhibits at least about 85% identity to a sequence selected from the group consisting of SEQ ID NOs: 31-44.

Disclosed herein include probes or primers up to about 100 nucleotides in length which is capable of hybridizing to the crtA gene of N. meningitidis. In some embodiments, the probe or primer comprises: a sequence selected from the group consisting of SEQ ID NOs: 45-59, or sequence that exhibits at least about 85% identity to a sequence selected from the group consisting of SEQ ID NOs: 45-59.

Disclosed herein include probes or primers up to about 100 nucleotides in length which is capable of hybridizing to an internal amplification control (IAC). In some embodiments, the probe or primer comprises: a sequence selected from the group consisting of SEQ ID NOs: 60-74 and 130-134, or sequence that exhibits at least about 85% identity to a sequence selected from the group consisting of SEQ ID NOs: 60-74 and 130-134.

Disclosed herein include probes or primers up to about 100 nucleotides in length which is capable of hybridizing to the nuc gene of S. aureus. In some embodiments, the probe or primer comprises: a sequence selected from the group consisting of SEQ ID NOs: 75-87, or sequence that exhibits at least about 85% identity to a sequence selected from the group consisting of SEQ ID NOs: 75-87.

Disclosed herein include probes or primers up to about 100 nucleotides in length which is capable of hybridizing to the fucK gene of H. influenzae. In some embodiments, the probe or primer comprises: a sequence selected from the group consisting of SEQ ID NOs: 88-100, or sequence that exhibits at least about 85% identity to a sequence selected from the group consisting of SEQ ID NOs: 88-100.

Disclosed herein include probes or primers up to about 100 nucleotides in length which is capable of hybridizing to the copB gene of M. catarrhalis. In some embodiments, the probe or primer comprises: a sequence selected from the group consisting of SEQ ID NOs: 101-115, or sequence that exhibits at least about 85% identity to a sequence selected from the group consisting of SEQ ID NOs: 101-115.

Disclosed herein include probes or primers up to about 100 nucleotides in length which is capable of hybridizing to the gltA gene of K. pneumoniae. In some embodiments, the probe or primer comprises: a sequence selected from the group consisting of SEQ ID NOs: 116-129, or sequence that exhibits at least about 85% identity to a sequence selected from the group consisting of SEQ ID NOs: 116-129.

Disclosed herein include compositions. In some embodiments, the composition comprises one or more, or two or more of the oligonucleotide probes and primers disclosed herein.

### Definitions

As used herein, the term “nucleic acid” can refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, “target nucleic acid”, and “target sequence” can be used interchangeably. As used herein, a “nucleic acid” can refer to a polymeric compound comprising nucleosides or nucleoside analogs which have nitrogenous heterocyclic bases, or base analogs, linked together by nucleic acid backbone linkages (e.g., phosphodiester bonds) to form a polynucleotide. Non-limiting examples of nucleic acid include RNA, DNA, and analogs thereof. The nucleic acid backbone can include a variety of linkages, for example, one or more of sugar-phosphodiester linkages, peptide-nucleic acid bonds, phosphorothioate or methylphosphonate linkages or mixtures of such linkages in a single oligonucleotide. Sugar moieties in the nucleic acid can be either ribose or deoxyribose, or similar compounds with known substitutions. Conventional nitrogenous bases (e.g., A, G, C, T, U), known base analogs (e.g., inosine), derivatives of purine or pyrimidine bases and “abasic” residues (i.e., no nitrogenous base for one or more backbone positions) are included in the term nucleic acid. That is, a nucleic acid can include only conventional sugars, bases and linkages found in RNA and DNA, or include both conventional components and substitutions (e.g., conventional bases and analogs linked via a methoxy backbone, or conventional bases and one or more base analogs linked via an RNA or DNA backbone).

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one).

As used herein, the term “isolate nucleic acids” can refer to the purification of nucleic acids from one or more cellular components. One of skill in the art will appreciate that samples processed to “isolate nucleic acids” therefrom can include components and impurities other than nucleic acids. Samples that comprise isolated nucleic acids can be prepared from specimens using any acceptable method known in the art. For example, cells can be lysed using known lysis agents, and nucleic acids can be purified or partially purified from other cellular components. Suitable reagents and protocols for DNA and RNA extractions can be found in, for example, U.S. Patent Application Publication Nos. US 2010-0009351, and US 2009-0131650, respectively (each of which is incorporated herein by reference in its entirety). In nucleic acid testing (e.g., amplification and hybridization methods discussed in further detail below), the extracted nucleic acid solution can be added directly to a reagents (e.g., either in liquid, bound to a substrate, in lyophilized form, or the like, as discussed in further detail below), required to perform a test according to the embodiments disclosed herein.

As used herein, “template” can refer to all or part of a polynucleotide containing at least one target nucleotide sequence.

As used herein, a “primer” can refer to a polynucleotide that can serve to initiate a nucleic acid chain extension reaction. The length of a primer can vary, for example, from about 5 to about 100 nucleotides, from about 10 to about 50 nucleotides, from about 15 to about 40 nucleotides, or from about 20 to about 30 nucleotides. The length of a primer can be about 10 nucleotides, about 20 nucleotides, about 25 nucleotides, about 30 nucleotides, about 35 nucleotides, about 40 nucleotides, about 50 nucleotides, about 75 nucleotides, about 100 nucleotides, or a range between any two of these values. In some embodiments, the primer has a length of 10 to about 50 nucleotides, i.e., 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, 41, 42, 43, 44, 45, 46, 47, 48, 49, 50, or more nucleotides. In some embodiments, the primer has a length of 18 to 32 nucleotides.

As used herein, a “probe” can refer to an polynucleotide that can hybridizes (e.g., specifically) to a target sequence in a nucleic acid, under conditions that allow hybridization, thereby allowing detection of the target sequence or amplified nucleic acid. A probe's “target” generally refers to a sequence within or a subset of an amplified nucleic acid sequence which hybridizes specifically to at least a portion of a probe oligomer by standard hydrogen bonding (i.e., base pairing). A probe may comprise target-specific sequences and other sequences that contribute to three-dimensional conformation of the probe. Sequences are “sufficiently complementary” if they allow stable hybridization in appropriate hybridization conditions of a probe oligomer to a target sequence that is not completely complementary to the probe's target-specific sequence. The length of a probe can vary, for example, from about 5 to about 100 nucleotides, from about 10 to about 50 nucleotides, from about 15 to about 40 nucleotides, or from about 20 to about 30 nucleotides. The length of a probe can be about 10 nucleotides, about 20 nucleotides, about 25 nucleotides, about 30 nucleotides, about 35 nucleotides, about 40 nucleotides, about 50 nucleotides, about 100 nucleotides, or a range between any two of these values. In some embodiments, the probe has a length of 10 to about 50 nucleotides. For example, the primers and or probes can be at least 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, 41, 42, 43, 44, 45, 46, 47, 48, 49, 50, or more nucleotides. In some embodiments, the probe can be non-sequence specific.

Preferably, the primers and/or probes can be between 8 and 45 nucleotides in length. For example, the primers and or probes can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. The primer and probe can be modified to contain additional nucleotides at the 5′ or the 3′ terminus, or both. One of skill in the art will appreciate that additional bases to the 3′ terminus of amplification primers (not necessarily probes) are generally complementary to the template sequence. The primer and probe sequences can also be modified to remove nucleotides at the 5′ or the 3′ terminus. One of skill in the art will appreciate that in order to function for amplification, the primers or probes will be of a minimum length and annealing temperature as disclosed herein.

Primers and probes can bind to their targets at an annealing temperature, which is a temperature less than the melting temperature (Tₘ). As used herein, “Tₘ” and “melting temperature” are interchangeable terms which refer to the temperature at which 50% of a population of double-stranded polynucleotide molecules becomes dissociated into single strands. The formulae for calculating the Tₘ of polynucleotides are well known in the art. For example, the Tₘ may be calculated by the following equation: Tₘ=69.3+0.41× (G+C)%-6-50/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.]. See, e.g., C. R. Newton et al. PCR, 2nd ed., Springer-Verlag (New York: 1997), p.24 (incorporated by reference in its entirety, herein). Other more sophisticated computations exist in the art, which take structural as well as sequence characteristics into account for the calculation of Tₘ. The melting temperature of an oligonucleotide can depend on complementarity between the oligonucleotide primer or probe and the binding sequence, and on salt conditions. In some embodiments, an oligonucleotide primer or probe provided herein has a Tₘ of less than about 90° C. in 50 mM KCl, 10 mM Tris-HCl buffer, for example about 89° C., 88, 87, 86, 85, 84, 83, 82, 81, 80 79, 78, 77, 76, 75, 74, 73, 72, 71, 70, 69, 68, 67, 66, 65, 64, 63, 62, 61, 60, 59, 58, 57, 56, 55, 54, 53, 52, 50, 49, 48, 47, 46, 45, 44, 43, 42, 41, 40, 39° C., or less, including ranges between any two of the listed values.

In some embodiments, the primers disclosed herein, e.g., amplification primers, can be provided as an amplification primer pair, e.g., comprising a forward primer and a reverse primer (first amplification primer and second amplification primer). Preferably, the forward and reverse primers have Tₘ's that do not differ by more than 10° C., e.g., that differ by less than 10° C., less than 9° C., less than 8° C., less than 7° C., less than 6° C., less than 5° C., less than 4° C., less than 3° C., less than 2° C., or less than 1C.

The primer and probe sequences may be modified by having nucleotide substitutions (relative to the target sequence) within the oligonucleotide sequence, provided that the oligonucleotide contains enough complementarity to hybridize specifically to the target nucleic acid sequence. In this manner, at least 1, 2, 3, 4, or up to about 5 nucleotides can be substituted. As used herein, the term “complementary” can refer to sequence complementarity between regions of two polynucleotide strands or between two regions of the same polynucleotide strand. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Fully complementary” can refer to a first polynucleotide that is 100% or “fully” complementary to a second polynucleotide and thus forms a base pair at every nucleotide position. “Partially complementary” also can refer to a first polynucleotide that is not 100% complementary (e.g., 90%, or 80% or 70% complementary) and contains mismatched nucleotides at one or more nucleotide positions. In some embodiments, an oligonucleotide includes a universal base.

As used herein, an “exogenous nucleotide sequence” can refer to a sequence introduced by primers or probes used for amplification, such that amplification products will contain exogenous nucleotide sequence and target nucleotide sequence in an arrangement not found in the original template from which the target nucleotide sequence was copied.

As used herein, “sequence identity” or “percent identical” as applied to nucleic acid molecules can refer to the percentage of nucleic acid residues in a candidate nucleic acid molecule sequence that are identical with a subject nucleic acid molecule sequence, after aligning the sequences to achieve the maximum percent identity, and not considering any nucleic acid residue substitutions as part of the sequence identity. Nucleic acid sequence identity can be determined using any method known in the art, for example CLUSTALW, T-COFFEE, BLASTN.

As used herein, the term “sufficiently complementary” can refer to a contiguous nucleic acid base sequence that is capable of hybridizing to another base sequence by hydrogen bonding between a series of complementary bases. Complementary base sequences can be complementary at each position in the oligomer sequence by using standard base pairing (e.g., G:C, A:T or A:U) or can contain one or more residues that are not complementary (including abasic positions), but in which the entire complementary base sequence is capable of specifically hybridizing with another base sequence in appropriate hybridization conditions. Contiguous bases can be at least about 80%, at least about 85%, at least about 90%, at least about 95%, at least about 99%, or 100% complementary to a sequence to which an oligomer is intended to hybridize. Substantially complementary sequences can refer to sequences ranging in percent identity from 100, 99, 98, 97, 96, 95, 94, 93, 92, 91, 90, 89, 88, 87, 86, 85, 84, 83, 82, 81, 80, 75, 70 or less, or any number in between, compared to the reference sequence. A skilled artisan can readily choose appropriate hybridization conditions which can be predicted based on base sequence composition, or be determined by using routine testing (see e.g., Green and Sambrook, Molecular Cloning, A Laboratory Manual, 4th ed. (Cold Spring Harbor Laboratory Press, Cold Spring Harbor, N.Y., 2012)).

As used herein, the term “multiplex PCR” refers to a type of PCR where more than one set of primers is included in a reaction allowing one single target, or two or more different targets, to be amplified in a single reaction vessel (e.g., tube). The multiplex PCR can be, for example, a real-time PCR.

### Oligonucleotides and Compositions Containing Thereof

As described herein, nucleic acid amplifications can be performed to determine the presence, absence, type, and/or level of bacterial respiratory pathogens in a sample, such as, for example, Streptococcus pneumoniae, Haemophilus influenzae, Staphylococcus aureus, Moraxella (Branhamella) catarrhalis, Neisseria meningitides, and Klebsiella pneumoniae. In some embodiments, the presence, absence and/or level of S. aureus, H. influenzae, M. catarrhalis, and K. pneumoniae and/or the presence, absence and/or level of S. pneumoniae and N. meningitidis is determined by detecting one or more target genes of each of the target organisms using methods known in the art, such as DNA amplifications. In some embodiments, a multiplex PCR can be performed to detect the presence, absence or level for each of S. aureus, H. influenzae, M. catarrhalis, and K. pneumoniae. In some embodiments, a multiplex PCR is performed to detect the presence, absence and/or level for each of S. pneumoniae and N. meningitidis.

In some embodiments, each of the target pathogenic bacteria disclosed herein can be detected using separate channels in DNA amplifications. In some embodiments, it can be desirable to use a single fluorescence channel for detecting the presence, absence, and/or level of two or more of the bacterial respiratory pathogens. For example, a single fluorescence channel can be used to detect the presence, absence, and/or level of two or more bacterial respiratory pathogens. Such combination may, in some embodiments, reduce the amount of reagent needed to conduct the experiment as well as provide an accurate qualitative metric upon which a respiratory tract infection determination can be assessed. Without being bound any particular theory, it is believed that the use of combined markers may increase the sensitivity and specificity of the assay.

There are provided, in some embodiments, two multiplex real-time PCR assays for the sensitive detection of S. aureus, H. influenzae, M. catarrhalis, and K. pneumoniae, and for the sensitive detection of S. pneumoniae and N. meningitidis, respectively. Disclosed herein include compositions and methods for multiplex real-time PCR assays enabling the rapid detection of common bacterial pathogens causing respiratory infection. In some embodiments, there are provided methods and reagents utilizing fluorogenic sequence-specific hybridization probes which provide a rapid and economical solution to: (1) identification of S. pneumoniae and N. meningitides (e.g., multiplex assay 1 in the Examples); (2) identification of H influenzae, S. aureus, M. catarrhalis and K. pneumoniae (e.g., multiplex assay 2 in the Examples); and (3) quality control of the DNA extraction and real-time PCR processes.

The disclosed methods can comprise: subjecting the DNA from a blood sample or culture suspected of containing above-mentioned 6 pathogens to a multiplex polymerase chain reaction amplification utilizing 2 groups of 5 sets concentration optimized primer pairs and probes; treating the reaction mixture under the optimum thermal condition, and detecting amplified DNA targets by monitor fluorescence signals of the hydrolysis (TaqMan®) probes at each cycle and interpreting the data at the end of the program to report the final results. The multiplex PCR primers and probes disclosed herein were designed and screened by using primer design software Primer 3 and Beacon Designer. Rapid and highly sensitive detection and discrimination the above-mentioned respiratory pathogens is achieved by the methodology herein provided.

The compositions and methods provided herein possess a number of advantages over currently available methods. First, S. pneumoniae, H. influenzae, S. aureus, M. catarrhalis, N. meningitides, and K. pneumoniae, which are the common pathogens in respiratory tract infection disease, can be detected simultaneously with only one sample. Second, the gene targets provided herein are optimized with higher sensitivity and inclusivity: (a) the two genes employed as targets for detection of N. meningitidis in species-specific assays, ctrA and sodC, enable the methods disclosed herein to achieve extraordinary performance by combining the high sensitivity of ctrA and the high inclusivity of sodC; (b) lytA is employed as a target for S. pneumoniae as it is superior to other targets, such as ply which is commonly used, and the disclosure adds the other well-characterized marker cpsA to realize better identification of S. pneumoniae; (c) for the identification of H. influenzae, the fucK target is employed instead of hpd gene because it has better sensitivity for the detection of H. influenzae and discrimination from H. haemolyticus in a recent reference laboratory-based study; and (d) the gltA gene is conserved in all three K. pneumoniae phylogroups called KpI, KpII, and KpIII, and therefore the disclosed methods can avoid missing detection of strains with important clinical significance. Third, the internal control disclosed herein can indicate false-negative results that are mainly caused by the PCR inhibitors, instrument or reagent failure, and the Ct value of IAC can be stable and not be influenced by the sample volume. Fourth, the primer/probe combinations and multiplex real-time PCR methods provided herein can achieve high sensitivity, inclusivity, and specificity, as demonstrated in the Examples. Moreover, the disclosed methods are both fast and easy to perform.

Oligonucleotides (for example amplification primers and probes) that are capable of specifically hybridizing (e.g., under standard nucleic acid amplification conditions, e.g., standard PCR conditions, and/or stringent hybridization conditions) to a target gene region, or complement thereof, in Streptococcus pneumoniae, Haemophilus influenzae, Staphylococcus aureus, Moraxella (Branhamella) catarrhalis, Neisseria meningitides, and Klebsiella pneumoniae are provided. Amplification of the target gene region of an organism in a sample (e.g., a blood or respiratory sample) can, in some embodiments, be indicative of the presence, absence, and/or level of the organism in the sample.

The target gene region can vary. In some embodiments, lytA is used as a marker for detecting S. pneumonia. The autolysin gene lytA is highly conserved within the species and has been shown that this assay best separates S. pneumoniae from the genotypically similar species S. mitis, S. oralis, and S. pseudopneumoniae. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding lytA in S. pneumoniae are provided. In some embodiments, lytA gene is used as the target gene for the DNA amplification to detect the presence, absence and/or level of S. pneumoniae in the sample. In some embodiments, primers and probes that can specifically bind to the lytA gene region of S. pneumoniae are used in detection of the presence, absence and/or level of S. pneumoniae in a biological sample. Examples of oligonucleotides capable of specifically hybridizing to the lytA gene region in S. pneumoniae include, but are not limited, SEQ ID NOs: 1-15 as provided in Table 1 and sequences that exhibits at least about 85% identity to a sequence selected from the group consisting of SEQ ID NOs: 1-15.

In some embodiments, cpsA is used as a marker for detecting S. pneumoniae. The capsular biosynthesis gene, cpsA, which can discriminate S. pneumoniae from the closely related viridans group streptococci as well as other pneumococcus-like streptococci such as S. pseudopneumoniae. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding cpsA in S. pneumoniae are provided. In some embodiments, cpsA gene is used as the target gene for the DNA amplification to detect the presence, absence and/or level of S. pneumoniae in the sample. In some embodiments, primers and probes that can specifically bind to the cpsA gene region of S. pneumoniae are used in detection of the presence, absence and/or level of S. pneumoniae in a biological sample. Examples of oligonucleotides capable of specifically hybridizing to the cpsA gene region in S. pneumoniae include, but are not limited, SEQ ID NOs: 16-30 as provided in Table 1 and sequences that exhibits at least about 85% identity to a sequence selected from the group consisting of SEQ ID NOs: 16-30.

In some embodiments, sodC is used as a marker for detecting N. meningitidis. The Cu—Zn superoxide dismutase gene sodC is found in N. meningitidis but not in other Neisseria species In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding sodC in N. meningitidis are provided. In some embodiments, sodC gene is used as the target gene for the DNA amplification to detect the presence, absence and/or level of N. meningitidis in the sample. In some embodiments, primers and probes that can specifically bind to the sodC gene region of N. meningitidis are used in detection of the presence, absence and/or level of N. meningitidis in a biological sample. Examples of oligonucleotides capable of specifically hybridizing to the sodC gene region in N. meningitidis include, but are not limited, SEQ ID NOs: 31-44 as provided in Table 1 and sequences that exhibits at least about 85% identity to a sequence selected from the group consisting of SEQ ID NOs: 31-44.

In some embodiments, ctrA is used as a marker for detecting N. meningitidis. ctrA encodes an outer membrane protein-encoding gene and is highly conserved among isolates responsible for invasive meningococcal infections. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding ctrA in N. meningitidis are provided. In some embodiments, ctrA gene is used as the target gene for the DNA amplification to detect the presence, absence and/or level of N. meningitidis in the sample. In some embodiments, primers and probes that can specifically bind to the ctrA gene region of N. meningitidis are used in detection of the presence, absence and/or level of N. meningitidis in a biological sample. Examples of oligonucleotides capable of specifically hybridizing to the ctrA gene region in N. meningitidis include, but are not limited, SEQ ID NOs: 45-59 as provided in Table 1 and sequences that exhibits at least about 85% identity to a sequence selected from the group consisting of SEQ ID NOs: 45-59.

In some embodiments, nuc is used as a marker for detecting S. aureus. The thermostable nuclease (nuc) has been successfully used to distinguish S. aureus from other Staphylococcus spp. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding nuc in S. aureus are provided. In some embodiments, nuc gene is used as the target gene for the DNA amplification to detect the presence, absence and/or level of S. aureus in the sample. In some embodiments, primers and probes that can specifically bind to the nuc gene region of S. aureus are used in detection of the presence, absence and/or level of S. aureus in a biological sample. Examples of oligonucleotides capable of specifically hybridizing to the nuc gene region in S. aureus include, but are not limited, SEQ ID NOs: 75-87 as provided in Table 3 and sequences that exhibits at least about 85% identity to a sequence selected from the group consisting of SEQ ID NOs: 75-87.

In some embodiments, fucK is used as a marker for detecting H. influenzae. The fuculokinase gene (fucK) codes for an enzyme involved in the metabolism of fucose and is the most highly conserved of the 7 housekeeping genes utilized in the multilocus sequence typing (MLST) scheme of H. influenzae. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding fucK in H. influenzae are provided. In some embodiments, fucK is used as the target gene for the DNA amplification to detect the presence, absence and/or level of H. influenzae in the sample. In some embodiments, primers and probes that can specifically bind to the fucK gene region of H. influenzae are used in detection of the presence, absence and/or level of H. influenzae in a biological sample. Examples of oligonucleotides capable of specifically hybridizing to the fucK gene region in H. influenzae include, but are not limited, SEQ ID NOs: 88-100 as provided in Table 3 and sequences that exhibits at least about 85% identity to a sequence selected from the group consisting of SEQ ID NOs: 88-100.

In some embodiments, copB is used as a marker for detecting M. catarrhalis. The copB (outer membrane protein) gene target is relatively conserved and present in every isolate examined and can be employed for the detection of M. catarrhalis. In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding copB in M. catarrhalis are provided. In some embodiments, copB is used as the target gene for the DNA amplification to detect the presence, absence and/or level of M. catarrhalis in the sample. In some embodiments, primers and probes that can specifically bind to the copB gene region of M. catarrhalis are used in detection of the presence, absence and/or level of M. catarrhalis in a biological sample. Examples of oligonucleotides capable of specifically hybridizing to the copB gene region in M. catarrhalis include, but are not limited, SEQ ID NOs: 101-115 as provided in Table 3 and sequences that exhibits at least about 85% identity to a sequence selected from the group consisting of SEQ ID NOs: 101-115.

In some embodiments, gltA is used as a marker for detecting K. pneumoniae. gltA is present in all three K. pneumoniae phylogroups called KpI, KpII (K. variicola) and KpIII (K. quasipneumoniae). In some embodiments, oligonucleotides (e.g., amplification primers and probes) that are capable of specifically hybridizing (e.g., under standard nucleic acid amplification conditions, e.g., standard PCR conditions, and/or stringent hybridization conditions) to a gene region encoding gltA in K. pneumoniae are provided. In some embodiments, gltA gene is used as the target gene for the DNA amplification to detect the presence, absence and/or level of K. pneumoniae in the sample. In some embodiments, primers and probes that can specifically bind to the gltA gene region of K. pneumoniae are used in detection of the presence, absence and/or level of K. pneumoniae in a biological sample. Examples of oligonucleotides capable of specifically hybridizing to the gltA gene region in K. pneumoniae include, but are not limited, SEQ ID NOs: 116-129 as provided in Table 3 and sequences that exhibits at least about 85% identity to a sequence selected from the group consisting of SEQ ID NOs: 116-129.

There is provided, in some embodiments, an internal amplification control (IAC). The IAC can be a sequence of no significant nucleotide identity to any known in the NCBI database and chosen as the marker of internal control. This internal control is used to indicate false-negative results that are mainly caused by the PCR inhibitors, instrument or reagent failure. The IAC can be used in nucleic acid amplification assays disclosed herein to determine if the test conditions are permissible for amplification and/or detection of a target nucleic acid sequence. The IAC may be incorporated into the methods and compositions provided herein to verify negative results and to identify potential inhibitory specimens or to facilitate quantification of organism load in a sample, such as but not limited to viruses, bacteria, and fungi. For diagnostic applications, simultaneous amplification and detection of two different DNA sequences, e.g., a pathogenic bacterium target sequence and the IAC target sequence, enable the use of an IAC. The primers and probes capable of hybridizing to the IAC provided herein can be useful in verifying negative results and in monitoring for specimens that inhibit the reaction. In some embodiments, an IAC is added to a sample to be tested. Although not intended to be bound by a particular mechanism of action, the presence of an IAC in the same reaction as the target sequence allows an amplification assay disclosed herein to detect the presence of reaction inhibitors and/or conditions that may indicate a false negative result. As used herein, a false negative result can refer to a result indicating no detection of a target sequence, however, such indication is not due to the absence of the target sequence in the sample, but is due to human error or a reaction condition, for example, the lack of a critical reaction element, or the existence of a reaction inhibitor, or an error in performing the assay. In some embodiments, an IAC sequence is designed so that its 3′ or 5′ end contains a common sequence with a target nucleic acid sequence. In some embodiments, an IAC sequence is designed so that its 3′ and 5′ end do not contain a common sequence with a target nucleic acid sequence. An IAC sequence can be designed to comprise a nucleic acid sequence that is different from a target nucleic acid sequence to be amplified, so that the detection of the IAC amplification products and the target sequence (or amplicons thereof) can be differentiated. In some embodiments, there are provided probes that bind an IAC amplicon but to not bind a target sequence amplicon. Examples of oligonucleotides capable of specifically hybridizing to an IAC include, but are not limited, SEQ ID NOs: 60-74 and 130-134 as provided in Table 2 and sequences that exhibits at least about 85% identity to a sequence selected from the group consisting of SEQ ID NOs: 60-74 and 130-134.

The IAC can comprise or be derived from a vector (e.g., pBluescript DNA). The IAC can comprise or be derived from a gene (e.g., HBB gene). The IAC can comprise or be derived from a non-gene-encoding sequence (e.g., a non-gene-encoding portion of pBluescript DNA). The IAC can comprise a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to one or more of the following sequences (SEQ ID NOs: 135-139):

(SEQ ID NO: 135)
CAGATCGATATGGGTGCCGTTCGAGCAGTTTAGCCCTAAATCACCCTACC
GGCAGACGTATGTCACATTCACCAGGGAGACGCTTGACATTGGATGCTGT
TGTGCGCCCTCAACAATGTAACGAATGGCTGCTTCGTCTCG;
(SEQ ID NO: 136)
GCTTCCTCGCTCACTGACTCGCTGCGCTCGGTCGTTCGGCTGCGGCGAGC
GGTATCAGCTCACTCAAAGGCGGTAATACGGTT;
(SEQ ID NO: 137)
AGTGCCACCTAAATTGTAAGCGTTAATATTTTGTTAAAATTCGCGTTAAA
TTTTTGTTAAATCAGCTCATTTTTTAACCAATAGGCCGAAATCGGCAAAA
TCCCTTATAAATCAAAAGAATAGACCGAGATAGGGTTGAGT;
(SEQ ID NO: 138)
GGATGAAGTTGGTGGTGAGGCCCTGGGCAGGTTGGTATCAAGGTTACAAG
ACAGGTTTAAGGAGACCAATAGAAACTGGGCATGTGGAGACAGAGAAGAC
TCTTGGGTTTCTGATAGGC;
and
(SEQ ID NO: 139)
ATGGCAAGAAAGTGCTCGGTGCCTTTAGTGATGGCCTGGCTCACCTGGAC
AACCTCAAGGGCACCTTTGCCACACTGAGTGAGCTGCAC.

There are provided, in some embodiments, compositions and methods wherein one or more of the primer/probe combinations provided herein (e.g., E1, E2, E3, E4, E5, E6, and/or E7) is used in combination with an IAC comprising a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to one or more of SEQ ID2NOs: 135-139.

TABLE 1
Non-limiting examples of primers and probes for detection of S. pneumoniae and N.
meningitidis

| Target | | | |
|---|---|---|---|
| Organism / | Primer / | | |
| Target | Probe | Primer / | |
| Gene | combination | Probe Name | Primer /Probe Sequences (5′-3′) |
| S. | A1 | A1-lytA-FP | CGTGAGCAGTTTAAGCATGATATTG (SEQ ID NO: 1) |
| pneumoniae / | A1 | A1-lytA-RP | AAGTAGTACCAAGTGCCATTGATT (SEQ ID NO: 2) |
| lytA | A1 | A1-lytA-probe | CTCAAACTTGTCTTTTGGATAAGAGCCGTC (SEQ ID |
| | | | NO: 11) |
| | | | (e.g., 5′ fluorophore: 6-FAM, 3′ quencher: BHQ1) |
| | A2 | A2-lytA-FP | GGCATTAGCCGTGAGCAGTTTA (SEQ ID NO: 3) |
| | A2 | A2-lytA-RP | CGTACCAGTAGCCAGTGTCATTC (SEQ ID NO: 4) |
| | A2 | A2-lytA-probe | TCTGCCAGCCTGTTTCAATCGTCAAGCCGTTCT (SEQ |
| | | | ID NO: 12) |
| | | | (e.g., 5′ fluorophore: 6-FAM, 3′ quencher: BHQ1) |
| | A3 | A3-lytA-FP | CGGATTATCACTGGCGGAAAG (SEQ ID NO: 5) |
| | A3 | A3-lytA-RP | GCACCATTATCAACAGGTCCTAC (SEQ ID NO: 6) |
| | A3 | A3-lytA-probe | CATGATGCAACCGTTCCCAACAATGTGCGAGA (SEQ |
| | | | ID NO: 13) |
| | | | (e.g., 5′ fluorophore: 6-FAM, 3′ quencher: BHQ1) |
| | A4 | A4-lytA-FP | AGGCTATATGCTTGCAGACCG (SEQ ID NO: 7) |
| | A4 | A4-lytA-RP | GTAGCCATTTCGCCTGAGTTG (SEQ ID NO: 8) |
| | A4 | A4-lytA-probe | ACCAGTACCAGTTGCCGTCTGTGTGCTTCCTCC (SEQ |
| | | | ID NO: 14) |
| | | | (e.g., 5′ fluorophore: 6-FAM, 3′ quencher: BHQ1) |
| | A5 | A5-lytA-FP | GGCTATATGCTTGCAGACCG (SEQ ID NO: 9) |
| | A5 | A5-lytA-RP | TCCAGCCTGTAGCCATTTCG (SEQ ID NO: 10) |
| | A5 | A5-lytA-probe | CTGAGTIGTCGAACCAGTACCAGTTGCCGTCT (SEQ |
| | | | ID NO: 15) |
| | | | (e.g., 5′ fluorophore: 6-FAM, 3′ quencher: BHQ1) |
| S. | B1 | B1-cpsA-FP | GGTGTTCTCTATCCTTGTCAGC (SEQ ID NO: 16) |
| pneumoniae / | B1 | B1- cpsA-RP | AGTCGCATTTAAACGATTGGTC (SEQ ID NO: 17) |
| cpsA | B1 | B1-cpsA- | CTGTGTCGCTCTTTGCAGTACAGCAGT (SEQ ID NO: |
| | | probe | 26) |
| | | | (e.g., 5′ fluorophore: VIC, 3′ quencher: BHQ1) |
| | B2 | B2-cpsA-FP | GCACCGACTGGGACTGATAA (SEQ ID NO: 18) |
| | B2 | B2-cpsA-RP | GCTGCCAAGTAAGACGAACTC (SEQ ID NO: 19) |
| | B2 | B2-cpsA- | TCGACCGTCAAATCGGTATTCTGACTTGACTTAA |
| | | probe | (SEQ ID NO: 27) |
| | | | (e.g., 5′ fluorophore: VIC, 3′ quencher: BHQ1) |
| | B3 | B3-cpsA-FP | TGGTGTTCTCTATCCTTGTCAGC (SEQ ID NO: 20) |
| | B3 | B3-cpsA-RP | CGCATTTAAACGATTGGTCAGTCC (SEQ ID NO: 21) |
| | B3 | B3-cpsA- | ACAAACTGCTGTACTGCAAAGAGCGACACA (SEQ ID |
| | | probe | NO: 28) |
| | | | (e.g., 5′ fluorophore: VIC, 3′ quencher: BHQ1) |
| | B4 | B4-cpsA-FP | CCCAGTAGGGAATGTCCATCTA (SEQ ID NO: 22) |
| | B4 | B4-cpsA-RP | GGTCACGGTCTCCATCGGCTA (SEQ ID NO: 23) |
| | B4 | B4-cpsA- | AGTAGCGTTCACGTACAAAACCTAGAGCCTGC (SEQ |
| | | probe | ID NO: 29) |
| | | | (e.g., 5′ fluorophore: VIC, 3′ quencher: BHQ1) |
| | B5 | B5-cpsA-FP | CCTAGTGGTAACTGCGTTAGTCC (SEQ ID NO: 24) |
| | B5 | B5-cpsA-RP | CCAACAAACTGCTGTACTGCAAAG (SEQ ID NO: 25) |
| | B5 | B5-cpsA- | CGACACAGAGCTGACAAGGATAGAGAACACCAAC |
| | | probe | (SEQ ID NO: 30) |
| | | | (e.g., 5′ fluorophore: VIC, 3′ quencher: BHQ1) |
| N. | C1 | C1-sodC-FP | GTGCAGTATGTTCAGTTGGTGTT (SEQ ID NO: 31) |
| meningitidis / | C1 | C1-sodC-RP | CTGGATCAAGTTGTTGCACTTTCA (SEQ ID NO: 32) |
| sodC | C1 | C1-sodC- | CGCAAGCACACGAGCATAATACGATACCT (SEQ ID |
| | | probe | NO: 40) |
| | | | (e.g., 5′ fluorophore: ROX, 3′ quencher: BHQ2) |
| | C2 | C2-sodC-FP | AGCACACGAGCATAATACGATAC (SEQ ID NO: 33) |
| | C2 | C2-sodC-RP | GTACCCACATCTTTGTTACCGTT (SEQ ID NO: 34) |
| | C2 | C2-sodC- | ACTGGATCAAGTTGTTGCACTTTCACTTCA (SEQ ID |
| | | probe | NO: 41) |
| | | | (e.g., 5′ fluorophore: ROX, 3′ quencher: BHQ2) |
| | C3 | C3-sodC-FP | AGTGCAGTATGTTCAGTTGGTG (SEQ ID NO: 35) |
| | C3 | C3-sodC-RP | CTGGATCAAGTTGTTGCACTTTCA (SEQ ID NO: 32) |
| | C3 | C3-sodC- | TGCGCAAGCACACGAGCATAATACGATAC (SEQ ID |
| | | probe | NO: 42) |
| | | | (e.g., 5′ fluorophore: ROX, 3′ quencher: BHQ2) |
| | C4 | C4-sodC-FP | GGTGATTTACCTGCATTAACTGT (SEQ ID NO: 36) |
| | C4 | C4-sodC-RP | TGTGGATCATAATAGAGTGACCG (SEQ ID NO: 37) |
| | C4 | C4-sodC- | TGCATGATGGCACAGCAACAAATCCTG (SEQ ID NO: |
| | | probe | 43) |
| | | | (5′ fluorophore: ROX, 3′ quencher: BHQ2) |
| | C5 | C5-sodC-FP | GTGCAGTATGTTCAGTTG (SEQ ID NO: 38) |
| | C5 | C5-sodC-RP | GACCATAGTTAGATTCAGTAATAG (SEQ ID NO: 39) |
| | C5 | C5-sodC- | CGCAAGCACACGAGCATAATACGA (SEQ ID NO: 44) |
| | | probe | (e.g., 5′ fluorophore: ROX, 3′ quencher: BHQ2) |
| N. | D1 | D1-ctrA-FP | GAACGTCAGGATAAATGGA (SEQ ID NO: 45) |
| meningitidis / | D1 | D1-ctrA-RP | GCATCAGCCATATTCACA (SEQ ID NO: 46) |
| crtA | D1 | D1-ctrA-probe | TACCGTTGGAATCTCTGCCTCACT (SEQ ID NO: 55) |
| | | | (e.g., 5′ fluorophore: CY5, 3′ quencher: BHQ-3) |
| | D2 | D2-ctrA-FP | GCCAGAGGCTTATCGCTTTC (SEQ ID NO: 47) |
| | D2 | D2-ctrA-RP | TGGCGTATAGCGGAACACAA (SEQ ID NO: 48) |
| | D2 | D2-ctrA-probe | CACACCACGCGCATCAGAACGGC (SEQ ID NO: 56) |
| | | | (e.g., 5′ fluorophore: CY5, 3′ quencher: BHQ-3) |
| | D3 | D3-ctrA-FP | CGCGTGGTGTGTTTGTGTT (SEQ ID NO: 49) |
| | D3 | D3-ctrA-RP | TGCCTCACTGCCATAACCTT (SEQ ID NO: 50) |
| | D3 | D3-ctrA-probe | TCCGCTATACGCCATTGGTGGAATTGCCG (SEQ ID |
| | | | NO: 57) |
| | | | (e.g., 5′ fluorophore: CY5, 3′ quencher: BHQ-3) |
| | D4 | D4-ctrA-FP | TGTGTTCCGCTATACGCCATT (SEQ ID NO: 51) |
| | D4 | D4-ctrA-RP | CTGCCTCACTGCCATAACCT (SEQ ID NO: 52) |
| | D4 | D4-ctrA-probe | AGCAATCCATTTATCCTGACGTTCTGCCG (SEQ ID |
| | | | NO: 58) |
| | | | (e.g., 5′ fluorophore: CY5, 3′ quencher: BHQ-3) |
| | D5 | D5-ctrA-FP | GCAGTGAGGCAGAGATTCCA (SEQ ID NO: 53) |
| | D5 | D5-ctrA-RP | GGCGAGAACACAAACGACAAA (SEQ ID NO: 54) |
| | D5 | D5-ctrA-probe | TTCTGCACTTCAGCCAACGGCGCATTC (SEQ ID NO: |
| | | | 59) |
| | | | (e.g., 5′ fluorophore: CY5, 3′ quencher: BHQ-3) |

TABLE 2
Non-limiting examples of primers and probes for detection of an Internal Amplification
Control (IAC)

| Target | Primer / | | |
|---|---|---|---|
| Organism / | Probe | Primer / | |
| Target Gene | combination | Probe Name | Primer /Probe Sequences (5′-3′) |
| Internal | E1 | E1-IAC-FP | CAGATCGATATGGGTGCCG (SEQ ID NO: 60) |
| Amplification | E1 | E1-IAC-RP | CGAGACGATGCAGCCATTC (SEQ ID NO: 61) |
| Control | E1 | E1-IAC-probe | TCTCATGCGTCTCCCTGGTGAATGTG (SEQ ID NO: 70) |
| (IAC) | | | (5′ fluorophore: CY5.5, 3′ quencher: BHQ3) |
| | E2 | E2-IAC-FP | CCTACCGTCTGCTGTCCAAA (SEQ ID NO: 62) |
| | E2 | E2-IAC-RP | GTCGTTGCGCGGATAAACAA (SEQ ID NO: 63) |
| | E2 | E2-IAC-probe | TGCCAACCATGGCAGCCATGTGTTACA (SEQ ID NO: |
| | | | 71) |
| | | | (5′ fluorophore: CY5.5, 3′ quencher: BHQ3) |
| | E3 | E3-IAC-FP | CAGATCGATATGGGTGCCG (SEQ ID NO: 60) |
| | E3 | E3-IAC-RP | CGAGACGAAGCAGCCATTC (SEQ ID NO: 130) |
| | E3 | E3-IAC-probe | TGTCAAGCGTCTCCCTGGTGAATGTG (SEQ ID NO: 133) |
| | E4 | E4-IAC-FP | GCTTCCTCGCTCACTGACTC (SEQ ID NO: 131) |
| | E4 | E4-IAC-RP | AACCGTATTACCGCCTTTGAGTG (SEQ ID NO: 132) |
| | E4 | E4-IAC-probe | TGATACCGCTCGCCGCAGCCGAACGA (SEQ ID NO: |
| | | | 134) |
| | E5 | E5-IAC-FP | AGTGCCACCTAAATTGTAAGC (SEQ ID NO: 64) |
| | E5 | E5-IAC-RP | ACTCAACCCTATCTCGGTCT (SEQ ID NO: 65) |
| | E5 | E5-IAC-probe | CCAATAGGCCGAAATCGGCAAAATCCCT (SEQ ID NO: |
| | | | 72) |
| | | | (5′ fluorophore: CY5.5, 3′ quencher: BHQ3) |
| | E6 | E6-IAC-FP | GGATGAAGTTGGTGGTGAGG (SEQ ID NO: 66) |
| | E6 | E6-IAC-RP | GCCTATCAGAAACCCAAGAGTC (SEQ ID NO: 67) |
| | E6 | E6-IAC-probe | TCTCTGTCTCCACATGCCCAGTTTCT (SEQ ID NO: 73) |
| | | | (5′ fluorophore: CY5.5, 3′ quencher: BHQ3) |
| | E7 | E7-IAC-FP | ATGGCAAGAAAGTGCTCGGTG (SEQ ID NO: 68) |
| | E7 | E7-IAC-RP | GTGCAGCTCACTCAGTGTGG (SEQ ID NO: 69) |
| | E7 | E7-IAC-probe | AGTGATGGCCTGGCTCACCTGGACAACC (SEQ ID NO: |
| | | | 74) |
| | | | (5′ fluorophore: CY5.5, 3′ quencher: BHQ3) |

TABLE 3
Non-limiting examples of primers and probes for detection of S. aureus, H. influenzae,
M. catarrhalis, and K. pneumoniae

| Target | | | |
|---|---|---|---|
| Organism / | Primer / | Primer / | |
| Target | Probe | Probe | |
| Gene | combination | Name | Primer /Probe Sequences (5′-3′) |
| S. aureus / | F1 | F1-nuc-FP | CAACCAATGACATTCAGACTATT (SEQ ID NO: 75) |
| nuc | F1 | F1-nuc-RP | CGACTTCAATTTTCTTTGCATTT (SEQ ID NO: 76) |
| | F1 | F1-nuc- | TTTCGTAAATGCACTTGCTTCAGGACCA (SEQ ID NO: 84) |
| | | probe | (e.g., 5′ FAM, BHQ1 on “T”, 3′ SpC6) |
| | F2 | F2-nuc-FP | GTGGTTCTGAAGATCCAA (SEQ ID NO: 77) |
| | F2 | F2-nuc-RP | GGTTGACCTTTGTACATTA (SEQ ID NO: 78) |
| | F2 | F2-nuc- | AACCGTATCACCATCAATCGCTT (SEQ ID NO: 85) |
| | | probe | (e.g., 5′ FAM, BHQ1 on “T”, 3′ SpC6) |
| | F3 | F3-nuc-FP | TGCGACTTTAATTAAAGCGATTGA (SEQ ID NO: 79) |
| | F3 | F3-nuc-RP | ACTCGACTTCAATTTTCTTTGCA (SEQ ID NO: 80) |
| | F3 | F3-nuc- | TTCGTAAATGCACTTGCTTCAGGACCA (SEQ ID NO: 86) |
| | | probe | (e.g., 5′ FAM, BHQ1 on “T”, 3′ SpC6) |
| | F4 | F4-nuc-FP | CAACCAATGACATTCAGACTA (SEQ ID NO: 81) |
| | F4 | F4-nuc-RP | CTTGCTTCAGGACCATATTTC (SEQ ID NO: 82) |
| | F4 | F4-nuc- | TGGTTGATACACCTGAAACAAAGCATC (SEQ ID NO: 87) |
| | | probe | (e.g., 5′ FAM, BHQ1 on “T”, 3′ SpC6) |
| | F5 | F5-nuc-FP | AGTGCAACTTCAACTAAA (SEQ ID NO: 83) |
| | F5 | F5-nuc-RP | GGTTGACCTTTGTACATTA (SEQ ID NO: 78) |
| | F5 | F5-nuc- | AACCGTATCACCATCAATCGCTT (SEQ ID NO: 85) |
| | | probe | (e.g., 5′ FAM, BHQ1 on “T”, 3′ SpC6) |
| H. | G1 | G1-fucK- | CGTCAATGCTCACTCAACGCTTA (SEQ ID NO: 88) |
| influenzae / | | FP | |
| fucK | G1 | G1-fucK- | CTGCATAACGCATAGGAGGGAAA (SEQ ID NO: 89) |
| | | RP | |
| | G1 | G1-fucK- | TGGTCAATTCACTACAGATCACACAATGGCGGGA (SEQ ID |
| | | probe | NO: 96) |
| | | | (e.g., 5′ fluorophore: VIC, 3′ quencher: BHQ1) |
| | G2 | G2-fucK - | GTTTGCTACTCGTGGAGAAAT (SEQ ID NO: 90) |
| | | FP | |
| | G2 | G2-fucK- | TGGAACTGACTGACTTGCTGTA (SEQ ID NO: 91) |
| | | RP | |
| | G2 | G2-fucK- | ATGTCAGCGCATTACAACATATGGCAAATCAAC (SEQ ID |
| | | probe | NO: 97) |
| | | | (e.g., 5′ fluorophore: VIC, 3′ quencher: BHQ1) |
| | G3 | G3-fucK- | CTCACTCAACGCTTAACTGGTCAA (SEQ ID NO: 92) |
| | | FP | |
| | G3 | G3-fucK- | CTGCATAACGCATAGGAGGGAAA (SEQ ID NO: 89) |
| | | RP | |
| | G3 | G3-fucK- | CACTACAGATCACACAATGGCGGGAACATCAATG (SEQ ID |
| | | probe | NO: 98) |
| | | | (e.g., 5′ fluorophore: VIC, 3′ quencher: BHQ1) |
| | G4 | G4-fucK- | TGCTCACTCAACGCTTAACTGG (SEQ ID NO: 93) |
| | | FP | |
| | G4 | G4-fucK- | CTGCATAACGCATAGGAGGGAAA (SEQ ID NO: 89) |
| | | RP | |
| | G4 | G4-fucK- | CAATTCACTACAGATCACACAATGGCGGGAACATC (SEQ ID |
| | | probe | NO: 99) |
| | | | (e.g., 5′ fluorophore: VIC, 3′ quencher: BHQ1) |
| | G5 | G5-fucK- | CCGTACCTGTCATTTCTTGTGGA (SEQ ID NO: 94) |
| | | FP | |
| | G5 | G5-fucK- | CGTGCTGGGTTCTAGCCATTAAG (SEQ ID NO: 95) |
| | | RP | |
| | G5 | G5-fucK- | CCAGGTGCCAGAACTTAACACAGGCTGATTCAG (SEQ ID |
| | | probe | NO: 100) |
| | | | (e.g., 5′ fluorophore: VIC, 3′ quencher: BHQ1) |
| M. | H1 | H1-copB- | GTCAGGTAGATGCCCTTGTCTCTTA (SEQ ID NO: 101) |
| catarrhalis / | | FP | |
| copB | H1 | H1-copB- | ACCACATCATTGCCCAACAGA (SEQ ID NO: 102) |
| | | RP | |
| | H1 | H1-copB- | TGGTGTACCCTTTACCGCCTTTATAGTCGCTGT (SEQ ID NO: |
| | | probe | 111) |
| | | | (e.g., 5′ fluorophore: ROX, 3′ quencher: BHQ2) |
| | H2 | H2-copB- | CATTCGTGGCGTGCGTGAAG (SEQ ID NO: 103) |
| | | FP | |
| | H2 | H2-copB- | CTGAAGCTGTTCGTCAGTACGTTT (SEQ ID NO: 104) |
| | | RP | |
| | H2 | H2-copB- | TTTGACTTCGCCAATCGTGCCTTGACGCTAGA (SEQ ID NO: |
| | | probe | 112) |
| | | | (e.g., 5′ fluorophore: ROX, 3′ quencher: BHQ2) |
| | H3 | H3-copB- | GCGTGCGTGAAGAGTTTGAC (SEQ ID NO: 105) |
| | | FP | |
| | H3 | H3-copB- | CCCTTGCCTGCATATTTGTTA (SEQ ID NO: 106) |
| | | RP | |
| | H3 | H3-copB- | TCGCCAATCGTGCCTTGACGCTAGATATAGAA (SEQ ID NO: |
| | | probe | 113) |
| | | | (e.g., 5′ fluorophore: ROX, 3′ quencher: BHQ2) |
| | H4 | H4-copB- | TGTGGTCAGCCATCTAAATGAAG (SEQ ID NO: 107) |
| | | FP | |
| | H4 | H4-copB- | TAGCGTCAAGGCACGATTG (SEQ ID NO: 108) |
| | | RP | |
| | H4 | H4-copB- | AGTCAAACTCTTCACGCACGCCACGA (SEQ ID NO: 114) |
| | | probe | (e.g., 5′ fluorophore: ROX, 3′ quencher: BHQ2) |
| | H5 | H5-copB- | ACAGGTCGTACATGGACAGC (SEQ ID NO: 109) |
| | | FP | |
| | H5 | H5-copB- | GGCATCTACCCCTTCAACCAA (SEQ ID NO: 110) |
| | | RP | |
| | H5 | H5-copB- | ATGTCGCCTATCGCCTGCCAAACCCAAG (SEQ ID NO: 115) |
| | | probe | (e.g., 5′ fluorophore: ROX, 3′ quencher: BHQ2) |
| K. | I1 | I1-gltA-FP | GCGATGAATCTGCCTTCGTC (SEQ ID NO: 116) |
| pneumoniae / | I1 | I1-gltA-RP | GCCTGGTGCATCTCGTTC (SEQ ID NO: 117) |
| gltA | I1 | I1-gltA- | AGTGCGCCACCCACCCGACCG (SEQ ID NO: 125) |
| | | probe | (e.g., 5′ fluorophore: CY5, 3′ quencher: BHQ-3) |
| | I2 | I2-gltA-FP | TCGAGCAGGCGATGAACC (SEQ ID NO: 118) |
| | I2 | 12-gltA-RP | GGCGACGCAGGCGAATAA (SEQ ID NO: 119) |
| | I2 | 12-gltA- | ATCCTGGTGCTGCATGCCGATCACG (SEQ ID NO: 126) |
| | | probe | (e.g., 5′ fluorophore: CY5, 3′ quencher: BHQ-3) |
| | I3 | I3-gltA-FP | TGCGAATGCTGGAGGAGATTGA (SEQ ID NO: 120) |
| | I3 | 13-gltA-RP | CCCGATGGCTGGTTTCACG (SEQ ID NO: 121) |
| | I3 | 13-gltA- | CGTCGATCAGGTTCCGGCGTTTCTGC (SEQ ID NO: 127) |
| | | probe | (e.g., 5′ fluorophore: CY5, 3′ quencher: BHQ-3) |
| | I4 | 14-gltA-FP | TGCTGCAGGTGGCGATG (SEQ ID NO: 122) |
| | I4 | 14-gltA-RP | CGACAGGCCATTGTCCACA (SEQ ID NO: 123) |
| | I4 | 14-gltA- | CCCTTGAGGACGTGGCGCTGACC (SEQ ID NO: 128) |
| | | probe | (e.g., 5′ fluorophore: CY5, 3′ quencher: BHQ-3) |
| | I5 | 15-gltA-FP | GCGATGAATCTGCCTTCGTC (SEQ ID NO: 116) |
| | 15 | I5-gltA-RP | TGGTGCATCTCGTTCCAGTG (SEQ ID NO: 124) |
| | 15 | 15-gltA- | CCACCCACCCGACCGTCCGACCGA (SEQ ID NO: 129) |
| | | probe | (e.g., 5′ fluorophore: CY5, 3′ quencher: BHQ-3) |

Also provided herein are oligonucleotides (for example amplification primers or probes) containing 1, 2, 3, 4 or more mismatches or universal nucleotides relative to SEQ ID NOs: 1-139 or the complement thereof, including oligonucleotides that are at least 80% identical (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to SEQ ID NOs: 1-139 or the complement thereof. In some embodiments, the oligonucleotide comprises a sequence selected from SEQ ID NO: 1-139. In some embodiments, the oligonucleotide comprises a sequence that is at least about 85% identical to a sequence selected from SEQ ID NO: 1-139. In some embodiments, the oligonucleotide consists of a sequence selected from SEQ ID NO: 1-139. In some embodiments, the oligonucleotide consists of a sequence that is at least about 85% identical or at least about 95% identical to a sequence selected from SEQ ID NO: 1-139.

There are provided, in some embodiments, primer and/or probe combinations. A primer/probe combination can comprise, for example, a forward primer, a reverse primer, and a probe (e.g., A3-lytA-FP, A3-lytA-RP, and A3-lytA-probe in tandem). The compositions and methods provided herein can comprise one or more of the primer/probe combinations provided in Tables 1-3. For example, a method or composition can comprise primer/probe combination A3 (e.g., A3-lytA-FP, A3-lytA-RP, and A3-lytA-probe in tandem). Disclosed herein include methods and compositions comprising two or more primer/probe combinations (e.g., multiplexed reactions). For example, a method or composition can comprise primer/probe combinations A3, B3, C3, and D3 (e.g., A3-lytA-FP, A3-lytA-RP, A3-lytA-probe, B3-cpsA-FP, B3-cpsA-RP, B3-cpsA-probe, C3-sodC-FP, C3-sodC-RP, C3-sodC-probe, D3-ctrA-FP, D3-ctrA-RP, and D3-ctrA-probe in tandem). Disclosed herein include methods and compositions comprising: (1) one or more primer/probe combinations capable of specifically hybridizing to the sequence of the lytA gene, or a complement thereof, of S. pneumoniae (e.g., A1, A2, A3, A4, and/or A5); (2) one or more primer/probe combinations capable of specifically hybridizing to the sequence of the cpsA gene, or a complement thereof, of S. pneumoniae (e.g., B1, B2, B3, B4, and/or B5); (3) one or more primer/probe combinations capable of specifically hybridizing to the sequence of the sodC gene, or a complement thereof, of N. meningitidis (e.g., C1, C2, C3, C4, and/or C5); (4) one or more primer/probe combinations capable of specifically hybridizing to the sequence of the crtA gene, or a complement thereof, of N. meningitidis (e.g., D1, D2, D3, D4, and/or D5); (5) one or more primer/probe combinations capable of specifically hybridizing to the sequence of an internal amplification control (IAC), or a complement thereof (e.g., E1, E2, E3, E4, E5, E6, and/or E7); (6) one or more primer/probe combinations capable of specifically hybridizing to the sequence of the nuc gene, or a complement thereof, of S. aureus (e.g., F1, F2, F3, F4, and/or F5); (7) one or more primer/probe combinations capable of specifically hybridizing to the sequence of the fucK gene, or a complement thereof, of H. influenzae (e.g., G1, G2, G3, G4, and/or G5); (8) one or more primer/probe combinations capable of specifically hybridizing to the sequence of the copB gene, or a complement thereof, of M. catarrhalis (e.g., H1, H2, H3, H4, and/or H5); and/or (9) one or more primer/probe combinations capable of specifically hybridizing to the sequence of the gltA gene, or a complement thereof, of K. pneumoniae (e.g., I1, I2, I3, 14, and/or I5). Disclosed herein include methods and compositions comprising one or more of the primer/probe combinations provided in Table 4. Disclosed herein include methods and compositions comprising one or more of the primer/probe combinations provided in Table 5. Disclosed herein include methods and compositions comprising one or more of the primer/probe combinations provided in Table 6. Disclosed herein also include methods and compositions comprising one or more of the primer/probe combinations provided in Table 7.

TABLE 4
Multiplexing of the Primer/Probe Combinations shown in Table
1 for detection of S. pneumoniae and N. meningitidis

(A1, B1, C1, D1), (A2, B1, C1, D1), (A3, B1, C1, D1), (A4, B1, C1, D1), (A5, B1, C1, D1), (A1, B2, C1, D1),
(A2, B2, C1, D1), (A3, B2, C1, D1), (A4, B2, C1, D1), (A5, B2, C1, D1), (A1, B3, C1, D1), (A2, B3, C1, D1),
(A3, B3, C1, D1), (A4, B3, C1, D1), (A5, B3, C1, D1), (A1, B4, C1, D1), (A2, B4, C1, D1), (A3, B4, C1, D1),
(A4, B4, C1, D1), (A5, B4, C1, D1), (A1, B1, C2, D1), (A2, B1, C2, D1), (A3, B1, C2, D1), (A4, B1, C2, D1),
(A5, B1, C2, D1), (A1, B2, C2, D1), (A2, B2, C2, D1), (A3, B2, C2, D1), (A4, B2, C2, D1), (A5, B2, C2, D1),
(A1, B3, C2, D1), (A2, B3, C2, D1), (A3, B3, C2, D1), (A4, B3, C2, D1), (A5, B3, C2, D1), (A1, B4, C2, D1),
(A2, B4, C2, D1), (A3, B4, C2, D1), (A4, B4, C2, D1), (A5, B4, C2, D1), (A1, B1, C1, D2), (A2, B1, C1, D2),
(A3, B1, C1, D2), (A4, B1, C1, D2), (A5, B1, C1, D2), (A1, B2, C1, D2), (A2, B2, C1, D2), (A3, B2, C1, D2),
(A4, B2, C1, D2), (A5, B2, C1, D2), (A1, B3, C1, D2), (A2, B3, C1, D2), (A3, B3, C1, D2), (A4, B3, C1, D2),
(A5, B3, C1, D2), (A1, B4, C1, D2), (A2, B4, C1, D2), (A3, B4, C1, D2), (A4, B4, C1, D2), (A5, B4, C1, D2),
(A1, B1, C2, D2), (A2, B1, C2, D2), (A3, B1, C2, D2), (A4, B1, C2, D2), (A5, B1, C2, D2), (A1, B2, C2, D2),
(A2, B2, C2, D2), (A3, B2, C2, D2), (A4, B2, C2, D2), (A5, B2, C2, D2), (A1, B3, C2, D2), (A2, B3, C2, D2),
(A3, B3, C2, D2), (A4, B3, C2, D2), (A5, B3, C2, D2), (A1, B4, C2, D2), (A2, B4, C2, D2), (A3, B4, C2, D2),
(A4, B4, C2, D2), (A5, B4, C2, D2), (A1, B1, C1, D3), (A2, B1, C1, D3), (A3, B1, C1, D3), (A4, B1, C1, D3),
(A5, B1, C1, D3), (A1, B2, C1, D3), (A2, B2, C1, D3), (A3, B2, C1, D3), (A4, B2, C1, D3), (A5, B2, C1, D3),
(A1, B3, C1, D3), (A2, B3, C1, D3), (A3, B3, C1, D3), (A4, B3, C1, D3), (A5, B3, C1, D3), (A1, B4, C1, D3),
(A2, B4, C1, D3), (A3, B4, C1, D3), (A4, B4, C1, D3), (A5, B4, C1, D3), (A1, B1, C2, D3), (A2, B1, C2, D3),
(A3, B1, C2, D3), (A4, B1, C2, D3), (A5, B1, C2, D3), (A1, B2, C2, D3), (A2, B2, C2, D3), (A3, B2, C2, D3),
(A4, B2, C2, D3), (A5, B2, C2, D3), (A1, B3, C2, D3), (A2, B3, C2, D3), (A3, B3, C2, D3), (A4, B3, C2, D3),
(A5, B3, C2, D3), (A1, B4, C2, D3), (A2, B4, C2, D3), (A3, B4, C2, D3), (A4, B4, C2, D3), (A5, B4, C2, D3),
(A1, B1, C1, D4), (A2, B1, C1, D4), (A3, B1, C1, D4), (A4, B1, C1, D4), (A5, B1, C1, D4), (A1, B2, C1, D4),
(A2, B2, C1, D4), (A3, B2, C1, D4), (A4, B2, C1, D4), (A5, B2, C1, D4), (A1, B3, C1, D4), (A2, B3, C1, D4),
(A3, B3, C1, D4), (A4, B3, C1, D4), (A5, B3, C1, D4), (A1, B4, C1, D4), (A2, B4, C1, D4), (A3, B4, C1, D4),
(A4, B4, C1, D4), (A5, B4, C1, D4), (A1, B1, C2, D4), (A2, B1, C2, D4), (A3, B1, C2, D4), (A4, B1, C2, D4),
(A5, B1, C2, D4), (A1, B2, C2, D4), (A2, B2, C2, D4), (A3, B2, C2, D4), (A4, B2, C2, D4), (A5, B2, C2, D4),
(A1, B3, C2, D4), (A2, B3, C2, D4), (A3, B3, C2, D4), (A4, B3, C2, D4), (A5, B3, C2, D4), (A1, B4, C2, D4),
(A2, B4, C2, D4), (A3, B4, C2, D4), (A4, B4, C2, D4), (A5, B4, C2, D4), (A1, B1, C1, D5), (A2, B1, C1, D5),
(A3, B1, C1, D5), (A4, B1, C1, D5), (A5, B1, C1, D5), (A1, B2, C1, D5), (A2, B2, C1, D5), (A3, B2, C1, D5),
(A4, B2, C1, D5), (A5, B2, C1, D5), (A1, B3, C1, D5), (A2, B3, C1, D5), (A3, B3, C1, D5), (A4, B3, C1, D5),
(A5, B3, C1, D5), (A1, B4, C1, D5), (A2, B4, C1, D5), (A3, B4, C1, D5), (A4, B4, C1, D5), (A5, B4, C1, D5),
(A1, B1, C2, D5), (A2, B1, C2, D5), (A3, B1, C2, D5), (A4, B1, C2, D5), (A5, B1, C2, D5), (A1, B2, C2, D5),
(A2, B2, C2, D5), (A3, B2, C2, D5), (A4, B2, C2, D5), (A5, B2, C2, D5), (A1, B3, C2, D5), (A2, B3, C2, D5),
(A3, B3, C2, D5), (A4, B3, C2, D5), (A5, B3, C2, D5), (A1, B4, C2, D5), (A2, B4, C2, D5), (A3, B4, C2, D5),
(A4, B4, C2, D5), (A5, B4, C2, D5), (A1, B1, C1, D1), (A2, B1, C1, D1), (A3, B1, C1, D1), (A4, B1, C1, D1),
(A5, B1, C1, D1), (A1, B2, C1, D1), (A2, B2, C1, D1), (A3, B2, C1, D1), (A4, B2, C1, D1), (A5, B2, C1, D1),
(A1, B3, C1, D1), (A2, B3, C1, D1), (A3, B3, C1, D1), (A4, B3, C1, D1), (A5, B3, C1, D1), (A1, B4, C1, D1),
(A2, B4, C1, D1), (A3, B4, C1, D1), (A4, B4, C1, D1), (A5, B4, C1, D1), (A1, B1, C2, D1), (A2, B1, C2, D1),
(A3, B1, C2, D1), (A4, B1, C2, D1), (A5, B1, C2, D1), (A1, B2, C2, D1), (A2, B2, C2, D1), (A3, B2, C2, D1),
(A4, B2, C2, D1), (A5, B2, C2, D1), (A1, B3, C2, D1), (A2, B3, C2, D1), (A3, B3, C2, D1), (A4, B3, C2, D1),
(A5, B3, C2, D1), (A1, B4, C2, D1), (A2, B4, C2, D1), (A3, B4, C2, D1), (A4, B4, C2, D1), (A5, B4, C2, D1),
(A1, B1, C1, D2), (A2, B1, C1, D2), (A3, B1, C1, D2), (A4, B1, C1, D2), (A5, B1, C1, D2), (A1, B2, C1, D2),
(A2, B2, C1, D2), (A3, B2, C1, D2), (A4, B2, C1, D2), (A5, B2, C1, D2), (A1, B3, C1, D2), (A2, B3, C1, D2),
(A3, B3, C1, D2), (A4, B3, C1, D2), (A5, B3, C1, D2), (A1, B4, C1, D2), (A2, B4, C1, D2), (A3, B4, C1, D2),
(A4, B4, C1, D2), (A5, B4, C1, D2), (A1, B1, C2, D2), (A2, B1, C2, D2), (A3, B1, C2, D2), (A4, B1, C2, D2),
(A5, B1, C2, D2), (A1, B2, C2, D2), (A2, B2, C2, D2), (A3, B2, C2, D2), (A4, B2, C2, D2), (A5, B2, C2, D2),
(A1, B3, C2, D2), (A2, B3, C2, D2), (A3, B3, C2, D2), (A4, B3, C2, D2), (A5, B3, C2, D2), (A1, B4, C2, D2),
(A2, B4, C2, D2), (A3, B4, C2, D2), (A4, B4, C2, D2), (A5, B4, C2, D2), (A1, B1, C1, D3), (A2, B1, C1, D3),
(A3, B1, C1, D3), (A4, B1, C1, D3), (A5, B1, C1, D3), (A1, B2, C1, D3), (A2, B2, C1, D3), (A3, B2, C1, D3),
(A4, B2, C1, D3), (A5, B2, C1, D3), (A1, B3, C1, D3), (A2, B3, C1, D3), (A3, B3, C1, D3), (A4, B3, C1, D3),
(A5, B3, C1, D3), (A1, B4, C1, D3), (A2, B4, C1, D3), (A3, B4, C1, D3), (A4, B4, C1, D3), (A5, B4, C1, D3),
(A1, B1, C2, D3), (A2, B1, C2, D3), (A3, B1, C2, D3), (A4, B1, C2, D3), (A5, B1, C2, D3), (A1, B2, C2, D3),
(A2, B2, C2, D3), (A3, B2, C2, D3), (A4, B2, C2, D3), (A5, B2, C2, D3), (A1, B3, C2, D3), (A2, B3, C2, D3),
(A3, B3, C2, D3), (A4, B3, C2, D3), (A5, B3, C2, D3), (A1, B4, C2, D3), (A2, B4, C2, D3), (A3, B4, C2, D3),
(A4, B4, C2, D3), (A5, B4, C2, D3), (A1, B1, C1, D4), (A2, B1, C1, D4), (A3, B1, C1, D4), (A4, B1, C1, D4),
(A5, B1, C1, D4), (A1, B2, C1, D4), (A2, B2, C1, D4), (A3, B2, C1, D4), (A4, B2, C1, D4), (A5, B2, C1, D4),
(A1, B3, C1, D4), (A2, B3, C1, D4), (A3, B3, C1, D4), (A4, B3, C1, D4), (A5, B3, C1, D4), (A1, B4, C1, D4),
(A2, B4, C1, D4), (A3, B4, C1, D4), (A4, B4, C1, D4), (A5, B4, C1, D4), (A1, B1, C2, D4), (A2, B1, C2, D4),
(A3, B1, C2, D4), (A4, B1, C2, D4), (A5, B1, C2, D4), (A1, B2, C2, D4), (A2, B2, C2, D4), (A3, B2, C2, D4),
(A4, B2, C2, D4), (A5, B2, C2, D4), (A1, B3, C2, D4), (A2, B3, C2, D4), (A3, B3, C2, D4), (A4, B3, C2, D4),
(A5, B3, C2, D4), (A1, B4, C2, D4), (A2, B4, C2, D4), (A3, B4, C2, D4), (A4, B4, C2, D4), (A5, B4, C2, D4),
(A1, B1, C1, D5), (A2, B1, C1, D5), (A3, B1, C1, D5), (A4, B1, C1, D5), (A5, B1, C1, D5), (A1, B2, C1, D5),
(A2, B2, C1, D5), (A3, B2, C1, D5), (A4, B2, C1, D5), (A5, B2, C1, D5), (A1, B3, C1, D5), (A2, B3, C1, D5),
(A3, B3, C1, D5), (A4, B3, C1, D5), (A5, B3, C1, D5), (A1, B4, C1, D5), (A2, B4, C1, D5), (A3, B4, C1, D5),
(A4, B4, C1, D5), (A5, B4, C1, D5), (A1, B1, C2, D5), (A2, B1, C2, D5), (A3, B1, C2, D5), (A4, B1, C2, D5),
(A5, B1, C2, D5), (A1, B2, C2, D5), (A2, B2, C2, D5), (A3, B2, C2, D5), (A4, B2, C2, D5), (A5, B2, C2, D5),
(A1, B3, C2, D5), (A2, B3, C2, D5), (A3, B3, C2, D5), (A4, B3, C2, D5), (A5, B3, C2, D5), (A1, B4, C2, D5),
(A2, B4, C2, D5), (A3, B4, C2, D5), (A4, B4, C2, D5), (A5, B4, C2, D5), (A1, B1, C1, D1), (A2, B1, C1, D1),
(A3, B1, C1, D1), (A4, B1, C1, D1), (A5, B1, C1, D1), (A1, B2, C1, D1), (A2, B2, C1, D1), (A3, B2, C1, D1),
(A4, B2, C1, D1), (A5, B2, C1, D1), (A1, B3, C1, D1), (A2, B3, C1, D1), (A3, B3, C1, D1), (A4, B3, C1, D1),
(A5, B3, C1, D1), (A1, B4, C1, D1), (A2, B4, C1, D1), (A3, B4, C1, D1), (A4, B4, C1, D1), (A5, B4, C1, D1),
(A1, B1, C2, D1), (A2, B1, C2, D1), (A3, B1, C2, D1), (A4, B1, C2, D1), (A5, B1, C2, D1), (A1, B2, C2, D1),
(A2, B2, C2, D1), (A3, B2, C2, D1), (A4, B2, C2, D1), (A5, B2, C2, D1), (A1, B3, C2, D1), (A2, B3, C2, D1),
(A3, B3, C2, D1), (A4, B3, C2, D1), (A5, B3, C2, D1), (A1, B4, C2, D1), (A2, B4, C2, D1), (A3, B4, C2, D1),
(A4, B4, C2, D1), (A5, B4, C2, D1), (A1, B1, C1, D2), (A2, B1, C1, D2), (A3, B1, C1, D2), (A4, B1, C1, D2),
(A5, B1, C1, D2), (A1, B2, C1, D2), (A2, B2, C1, D2), (A3, B2, C1, D2), (A4, B2, C1, D2), (A5, B2, C1, D2),
(A1, B3, C1, D2), (A2, B3, C1, D2), (A3, B3, C1, D2), (A4, B3, C1, D2), (A5, B3, C1, D2), (A1, B4, C1, D2),
(A2, B4, C1, D2), (A3, B4, C1, D2), (A4, B4, C1, D2), (A5, B4, C1, D2), (A1, B1, C2, D2), (A2, B1, C2, D2),
(A3, B1, C2, D2), (A4, B1, C2, D2), (A5, B1, C2, D2), (A1, B2, C2, D2), (A2, B2, C2, D2), (A3, B2, C2, D2),
(A4, B2, C2, D2), (A5, B2, C2, D2), (A1, B3, C2, D2), (A2, B3, C2, D2), (A3, B3, C2, D2), (A4, B3, C2, D2),
(A5, B3, C2, D2), (A1, B4, C2, D2), (A2, B4, C2, D2), (A3, B4, C2, D2), (A4, B4, C2, D2), (A5, B4, C2, D2),
(A1, B1, C1, D3), (A2, B1, C1, D3), (A3, B1, C1, D3), (A4, B1, C1, D3), (A5, B1, C1, D3), (A1, B2, C1, D3),
(A2, B2, C1, D3), (A3, B2, C1, D3), (A4, B2, C1, D3), (A5, B2, C1, D3), (A1, B3, C1, D3), (A2, B3, C1, D3),
(A3, B3, C1, D3), (A4, B3, C1, D3), (A5, B3, C1, D3), (A1, B4, C1, D3), (A2, B4, C1, D3), (A3, B4, C1, D3),
(A4, B4, C1, D3), (A5, B4, C1, D3), (A1, B1, C2, D3), (A2, B1, C2, D3), (A3, B1, C2, D3), (A4, B1, C2, D3),
(A5, B1, C2, D3), (A1, B2, C2, D3), (A2, B2, C2, D3), (A3, B2, C2, D3), (A4, B2, C2, D3), (A5, B2, C2, D3),
(A1, B3, C2, D3), (A2, B3, C2, D3), (A3, B3, C2, D3), (A4, B3, C2, D3), (A5, B3, C2, D3), (A1, B4, C2, D3),
(A2, B4, C2, D3), (A3, B4, C2, D3), (A4, B4, C2, D3), (A5, B4, C2, D3), (A1, B1, C1, D4), (A2, B1, C1, D4),
(A3, B1, C1, D4), (A4, B1, C1, D4), (A5, B1, C1, D4), (A1, B2, C1, D4), (A2, B2, C1, D4), (A3, B2, C1, D4),
(A4, B2, C1, D4), (A5, B2, C1, D4), (A1, B3, C1, D4), (A2, B3, C1, D4), (A3, B3, C1, D4), (A4, B3, C1, D4),
(A5, B3, C1, D4), (A1, B4, C1, D4), (A2, B4, C1, D4), (A3, B4, C1, D4), (A4, B4, C1, D4), (A5, B4, C1, D4),
(A1, B1, C2, D4), (A2, B1, C2, D4), (A3, B1, C2, D4), (A4, B1, C2, D4), (A5, B1, C2, D4), (A1, B2, C2, D4),
(A2, B2, C2, D4), (A3, B2, C2, D4), (A4, B2, C2, D4), (A5, B2, C2, D4), (A1, B3, C2, D4), (A2, B3, C2, D4),
(A3, B3, C2, D4), (A4, B3, C2, D4), (A5, B3, C2, D4), (A1, B4, C2, D4), (A2, B4, C2, D4), (A3, B4, C2, D4),
(A4, B4, C2, D4), (A5, B4, C2, D4), (A1, B1, C1, D5), (A2, B1, C1, D5), (A3, B1, C1, D5), (A4, B1, C1, D5),
(A5, B1, C1, D5), (A1, B2, C1, D5), (A2, B2, C1, D5), (A3, B2, C1, D5), (A4, B2, C1, D5), (A5, B2, C1, D5),
(A1, B3, C1, D5), (A2, B3, C1, D5), (A3, B3, C1, D5), (A4, B3, C1, D5), (A5, B3, C1, D5), (A1, B4, C1, D5),
(A2, B4, C1, D5), (A3, B4, C1, D5), (A4, B4, C1, D5), (A5, B4, C1, D5), (A1, B1, C2, D5), (A2, B1, C2, D5),
(A3, B1, C2, D5), (A4, B1, C2, D5), (A5, B1, C2, D5), (A1, B2, C2, D5), (A2, B2, C2, D5), (A3, B2, C2, D5),
(A4, B2, C2, D5), (A5, B2, C2, D5), (A1, B3, C2, D5), (A2, B3, C2, D5), (A3, B3, C2, D5), (A4, B3, C2, D5),
(A5, B3, C2, D5), (A1, B4, C2, D5), (A2, B4, C2, D5), (A3, B4, C2, D5), (A4, B4, C2, D5), (A5, B4, C2, D5),
(A1, B1, C1, D1), (A2, B1, C1, D1), (A3, B1, C1, D1), (A4, B1, C1, D1), (A5, B1, C1, D1), (A1, B2, C1, D1),
(A2, B2, C1, D1), (A3, B2, C1, D1), (A4, B2, C1, D1), (A5, B2, C1, D1), (A1, B3, C1, D1), (A2, B3, C1, D1),
(A3, B3, C1, D1), (A4, B3, C1, D1), (A5, B3, C1, D1), (A1, B4, C1, D1), (A2, B4, C1, D1), (A3, B4, C1, D1),
(A4, B4, C1, D1), (A5, B4, C1, D1), (A1, B1, C2, D1), (A2, B1, C2, D1), (A3, B1, C2, D1), (A4, B1, C2, D1),
(A5, B1, C2, D1), (A1, B2, C2, D1), (A2, B2, C2, D1), (A3, B2, C2, D1), (A4, B2, C2, D1), (A5, B2, C2, D1),
(A1, B3, C2, D1), (A2, B3, C2, D1), (A3, B3, C2, D1), (A4, B3, C2, D1), (A5, B3, C2, D1), (A1, B4, C2, D1),
(A2, B4, C2, D1), (A3, B4, C2, D1), (A4, B4, C2, D1), (A5, B4, C2, D1), (A1, B1, C1, D2), (A2, B1, C1, D2),
(A3, B1, C1, D2), (A4, B1, C1, D2), (A5, B1, C1, D2), (A1, B2, C1, D2), (A2, B2, C1, D2), (A3, B2, C1, D2),
(A4, B2, C1, D2), (A5, B2, C1, D2), (A1, B3, C1, D2), (A2, B3, C1, D2), (A3, B3, C1, D2), (A4, B3, C1, D2),
(A5, B3, C1, D2), (A1, B4, C1, D2), (A2, B4, C1, D2), (A3, B4, C1, D2), (A4, B4, C1, D2), (A5, B4, C1, D2),
(A1, B1, C2, D2), (A2, B1, C2, D2), (A3, B1, C2, D2), (A4, B1, C2, D2), (A5, B1, C2, D2), (A1, B2, C2, D2),
(A2, B2, C2, D2), (A3, B2, C2, D2), (A4, B2, C2, D2), (A5, B2, C2, D2), (A1, B3, C2, D2), (A2, B3, C2, D2),
(A3, B3, C2, D2), (A4, B3, C2, D2), (A5, B3, C2, D2), (A1, B4, C2, D2), (A2, B4, C2, D2), (A3, B4, C2, D2),
(A4, B4, C2, D2), (A5, B4, C2, D2), (A1, B1, C1, D3), (A2, B1, C1, D3), (A3, B1, C1, D3), (A4, B1, C1, D3),
(A5, B1, C1, D3), (A1, B2, C1, D3), (A2, B2, C1, D3), (A3, B2, C1, D3), (A4, B2, C1, D3), (A5, B2, C1, D3),
(A1, B3, C1, D3), (A2, B3, C1, D3), (A3, B3, C1, D3), (A4, B3, C1, D3), (A5, B3, C1, D3), (A1, B4, C1, D3),
(A2, B4, C1, D3), (A3, B4, C1, D3), (A4, B4, C1, D3), (A5, B4, C1, D3), (A1, B1, C2, D3), (A2, B1, C2, D3),
(A3, B1, C2, D3), (A4, B1, C2, D3), (A5, B1, C2, D3), (A1, B2, C2, D3), (A2, B2, C2, D3), (A3, B2, C2, D3),
(A4, B2, C2, D3), (A5, B2, C2, D3), (A1, B3, C2, D3), (A2, B3, C2, D3), (A3, B3, C2, D3), (A4, B3, C2, D3),
(A5, B3, C2, D3), (A1, B4, C2, D3), (A2, B4, C2, D3), (A3, B4, C2, D3), (A4, B4, C2, D3), (A5, B4, C2, D3),
(A1, B1, C1, D4), (A2, B1, C1, D4), (A3, B1, C1, D4), (A4, B1, C1, D4), (A5, B1, C1, D4), (A1, B2, C1, D4),
(A2, B2, C1, D4), (A3, B2, C1, D4), (A4, B2, C1, D4), (A5, B2, C1, D4), (A1, B3, C1, D4), (A2, B3, C1, D4),
(A3, B3, C1, D4), (A4, B3, C1, D4), (A5, B3, C1, D4), (A1, B4, C1, D4), (A2, B4, C1, D4), (A3, B4, C1, D4),
(A4, B4, C1, D4), (A5, B4, C1, D4), (A1, B1, C2, D4), (A2, B1, C2, D4), (A3, B1, C2, D4), (A4, B1, C2, D4),
(A5, B1, C2, D4), (A1, B2, C2, D4), (A2, B2, C2, D4), (A3, B2, C2, D4), (A4, B2, C2, D4), (A5, B2, C2, D4),
(A1, B3, C2, D4), (A2, B3, C2, D4), (A3, B3, C2, D4), (A4, B3, C2, D4), (A5, B3, C2, D4), (A1, B4, C2, D4),
(A2, B4, C2, D4), (A3, B4, C2, D4), (A4, B4, C2, D4), (A5, B4, C2, D4), (A1, B1, C1, D5), (A2, B1, C1, D5),
(A3, B1, C1, D5), (A4, B1, C1, D5), (A5, B1, C1, D5), (A1, B2, C1, D5), (A2, B2, C1, D5), (A3, B2, C1, D5),
(A4, B2, C1, D5), (A5, B2, C1, D5), (A1, B3, C1, D5), (A2, B3, C1, D5), (A3, B3, C1, D5), (A4, B3, C1, D5),
(A5, B3, C1, D5), (A1, B4, C1, D5), (A2, B4, C1, D5), (A3, B4, C1, D5), (A4, B4, C1, D5), (A5, B4, C1, D5),
(A1, B1, C2, D5), (A2, B1, C2, D5), (A3, B1, C2, D5), (A4, B1, C2, D5), (A5, B1, C2, D5), (A1, B2, C2, D5),
(A2, B2, C2, D5), (A3, B2, C2, D5), (A4, B2, C2, D5), (A5, B2, C2, D5), (A1, B3, C2, D5), (A2, B3, C2, D5),
(A3, B3, C2, D5), (A4, B3, C2, D5), (A5, B3, C2, D5), (A1, B4, C2, D5), (A2, B4, C2, D5), (A3, B4, C2, D5),
(A4, B4, C2, D5), (A5, B4, C2, D5), (A1, B1, C1, D1), (A2, B1, C1, D1), (A3, B1, C1, D1), (A4, B1, C1, D1),
(A5, B1, C1, D1), (A1, B2, C1, D1), (A2, B2, C1, D1), (A3, B2, C1, D1), (A4, B2, C1, D1), (A5, B2, C1, D1),
(A1, B3, C1, D1), (A2, B3, C1, D1), (A3, B3, C1, D1), (A4, B3, C1, D1), (A5, B3, C1, D1), (A1, B4, C1, D1),
(A2, B4, C1, D1), (A3, B4, C1, D1), (A4, B4, C1, D1), (A5, B4, C1, D1), (A1, B1, C2, D1), (A2, B1, C2, D1),
(A3, B1, C2, D1), (A4, B1, C2, D1), (A5, B1, C2, D1), (A1, B2, C2, D1), (A2, B2, C2, D1), (A3, B2, C2, D1),
(A4, B2, C2, D1), (A5, B2, C2, D1), (A1, B3, C2, D1), (A2, B3, C2, D1), (A3, B3, C2, D1), (A4, B3, C2, D1),
(A5, B3, C2, D1), (A1, B4, C2, D1), (A2, B4, C2, D1), (A3, B4, C2, D1), (A4, B4, C2, D1), (A5, B4, C2, D1),
(A1, B1, C1, D2), (A2, B1, C1, D2), (A3, B1, C1, D2), (A4, B1, C1, D2), (A5, B1, C1, D2), (A1, B2, C1, D2),
(A2, B2, C1, D2), (A3, B2, C1, D2), (A4, B2, C1, D2), (A5, B2, C1, D2), (A1, B3, C1, D2), (A2, B3, C1, D2),
(A3, B3, C1, D2), (A4, B3, C1, D2), (A5, B3, C1, D2), (A1, B4, C1, D2), (A2, B4, C1, D2), (A3, B4, C1, D2),
(A4, B4, C1, D2), (A5, B4, C1, D2), (A1, B1, C2, D2), (A2, B1, C2, D2), (A3, B1, C2, D2), (A4, B1, C2, D2),
(A5, B1, C2, D2), (A1, B2, C2, D2), (A2, B2, C2, D2), (A3, B2, C2, D2), (A4, B2, C2, D2), (A5, B2, C2, D2),
(A1, B3, C2, D2), (A2, B3, C2, D2), (A3, B3, C2, D2), (A4, B3, C2, D2), (A5, B3, C2, D2), (A1, B4, C2, D2),
(A2, B4, C2, D2), (A3, B4, C2, D2), (A4, B4, C2, D2), (A5, B4, C2, D2), (A1, B1, C1, D3), (A2, B1, C1, D3),
(A3, B1, C1, D3), (A4, B1, C1, D3), (A5, B1, C1, D3), (A1, B2, C1, D3), (A2, B2, C1, D3), (A3, B2, C1, D3),
(A4, B2, C1, D3), (A5, B2, C1, D3), (A1, B3, C1, D3), (A2, B3, C1, D3), (A3, B3, C1, D3), (A4, B3, C1, D3),
(A5, B3, C1, D3), (A1, B4, C1, D3), (A2, B4, C1, D3), (A3, B4, C1, D3), (A4, B4, C1, D3), (A5, B4, C1, D3),
(A1, B1, C2, D3), (A2, B1, C2, D3), (A3, B1, C2, D3), (A4, B1, C2, D3), (A5, B1, C2, D3), (A1, B2, C2, D3),
(A2, B2, C2, D3), (A3, B2, C2, D3), (A4, B2, C2, D3), (A5, B2, C2, D3), (A1, B3, C2, D3), (A2, B3, C2, D3),
(A3, B3, C2, D3), (A4, B3, C2, D3), (A5, B3, C2, D3), (A1, B4, C2, D3), (A2, B4, C2, D3), (A3, B4, C2, D3),
(A4, B4, C2, D3), (A5, B4, C2, D3), (A1, B1, C1, D4), (A2, B1, C1, D4), (A3, B1, C1, D4), (A4, B1, C1, D4),
(A5, B1, C1, D4), (A1, B2, C1, D4), (A2, B2, C1, D4), (A3, B2, C1, D4), (A4, B2, C1, D4), (A5, B2, C1, D4),
(A1, B3, C1, D4), (A2, B3, C1, D4), (A3, B3, C1, D4), (A4, B3, C1, D4), (A5, B3, C1, D4), (A1, B4, C1, D4),
(A2, B4, C1, D4), (A3, B4, C1, D4), (A4, B4, C1, D4), (A5, B4, C1, D4), (A1, B1, C2, D4), (A2, B1, C2, D4),
(A3, B1, C2, D4), (A4, B1, C2, D4), (A5, B1, C2, D4), (A1, B2, C2, D4), (A2, B2, C2, D4), (A3, B2, C2, D4),
(A4, B2, C2, D4), (A5, B2, C2, D4), (A1, B3, C2, D4), (A2, B3, C2, D4), (A3, B3, C2, D4), (A4, B3, C2, D4),
(A5, B3, C2, D4), (A1, B4, C2, D4), (A2, B4, C2, D4), (A3, B4, C2, D4), (A4, B4, C2, D4), (A5, B4, C2, D4),
(A1, B1, C1, D5), (A2, B1, C1, D5), (A3, B1, C1, D5), (A4, B1, C1, D5), (A5, B1, C1, D5), (A1, B2, C1, D5),
(A2, B2, C1, D5), (A3, B2, C1, D5), (A4, B2, C1, D5), (A5, B2, C1, D5), (A1, B3, C1, D5), (A2, B3, C1, D5),
(A3, B3, C1, D5), (A4, B3, C1, D5), (A5, B3, C1, D5), (A1, B4, C1, D5), (A2, B4, C1, D5), (A3, B4, C1, D5),
(A4, B4, C1, D5), (A5, B4, C1, D5), (A1, B1, C2, D5), (A2, B1, C2, D5), (A3, B1, C2, D5), (A4, B1, C2, D5),
(A5, B1, C2, D5), (A1, B2, C2, D5), (A2, B2, C2, D5), (A3, B2, C2, D5), (A4, B2, C2, D5), (A5, B2, C2, D5),
(A1, B3, C2, D5), (A2, B3, C2, D5), (A3, B3, C2, D5), (A4, B3, C2, D5), (A5, B3, C2, D5), (A1, B4, C2, D5),
(A2, B4, C2, D5), (A3, B4, C2, D5), (A4, B4, C2, D5), and/or (A5, B4, C2, D5).

TABLE 5
Multiplexing of the Primer/Probe Combinations shown in Tables 1-2 for detection of
S. pneumoniae, N. meningitidis, and an internal amplification control (IAC)

(A1, B1, C1, D1, E1), (A2, B1, C1, D1, E1), (A3, B1, C1, D1, E1), (A4, B1, C1, D1, E1), (A5, B1, C1, D1, E1),
(A1, B2, C1, D1, E1), (A2, B2, C1, D1, E1), (A3, B2, C1, D1, E1), (A4, B2, C1, D1, E1), (A5, B2, C1, D1, E1),
(A1, B3, C1, D1, E1), (A2, B3, C1, D1, E1), (A3, B3, C1, D1, E1), (A4, B3, C1, D1, E1), (A5, B3, C1, D1, E1),
(A1, B4, C1, D1, E1), (A2, B4, C1, D1, E1), (A3, B4, C1, D1, E1), (A4, B4, C1, D1, E1), (A5, B4, C1, D1, E1),
(A1, B1, C2, D1, E1), (A2, B1, C2, D1, E1), (A3, B1, C2, D1, E1), (A4, B1, C2, D1, E1), (A5, B1, C2, D1, E1),
(A1, B2, C2, D1, E1), (A2, B2, C2, D1, E1), (A3, B2, C2, D1, E1), (A4, B2, C2, D1, E1), (A5, B2, C2, D1, E1),
(A1, B3, C2, D1, E1), (A2, B3, C2, D1, E1), (A3, B3, C2, D1, E1), (A4, B3, C2, D1, E1), (A5, B3, C2, D1, E1),
(A1, B4, C2, D1, E1), (A2, B4, C2, D1, E1), (A3, B4, C2, D1, E1), (A4, B4, C2, D1, E1), (A5, B4, C2, D1, E1),
(A1, B1, C1, D2, E1), (A2, B1, C1, D2, E1), (A3, B1, C1, D2, E1), (A4, B1, C1, D2, E1), (A5, B1, C1, D2, E1),
(A1, B2, C1, D2, E1), (A2, B2, C1, D2, E1), (A3, B2, C1, D2, E1), (A4, B2, C1, D2, E1), (A5, B2, C1, D2, E1),
(A1, B3, C1, D2, E1), (A2, B3, C1, D2, E1), (A3, B3, C1, D2, E1), (A4, B3, C1, D2, E1), (A5, B3, C1, D2, E1),
(A1, B4, C1, D2, E1), (A2, B4, C1, D2, E1), (A3, B4, C1, D2, E1), (A4, B4, C1, D2, E1), (A5, B4, C1, D2, E1),
(A1, B1, C2, D2, E1), (A2, B1, C2, D2, E1), (A3, B1, C2, D2, E1), (A4, B1, C2, D2, E1), (A5, B1, C2, D2, E1),
(A1, B2, C2, D2, E1), (A2, B2, C2, D2, E1), (A3, B2, C2, D2, E1), (A4, B2, C2, D2, E1), (A5, B2, C2, D2, E1),
(A1, B3, C2, D2, E1), (A2, B3, C2, D2, E1), (A3, B3, C2, D2, E1), (A4, B3, C2, D2, E1), (A5, B3, C2, D2, E1),
(A1, B4, C2, D2, E1), (A2, B4, C2, D2, E1), (A3, B4, C2, D2, E1), (A4, B4, C2, D2, E1), (A5, B4, C2, D2, E1),
(A1, B1, C1, D3, E1), (A2, B1, C1, D3, E1), (A3, B1, C1, D3, E1), (A4, B1, C1, D3, E1), (A5, B1, C1, D3, E1),
(A1, B2, C1, D3, E1), (A2, B2, C1, D3, E1), (A3, B2, C1, D3, E1), (A4, B2, C1, D3, E1), (A5, B2, C1, D3, E1),
(A1, B3, C1, D3, E1), (A2, B3, C1, D3, E1), (A3, B3, C1, D3, E1), (A4, B3, C1, D3, E1), (A5, B3, C1, D3, E1),
(A1, B4, C1, D3, E1), (A2, B4, C1, D3, E1), (A3, B4, C1, D3, E1), (A4, B4, C1, D3, E1), (A5, B4, C1, D3, E1),
(A1, B1, C2, D3, E1), (A2, B1, C2, D3, E1), (A3, B1, C2, D3, E1), (A4, B1, C2, D3, E1), (A5, B1, C2, D3, E1),
(A1, B2, C2, D3, E1), (A2, B2, C2, D3, E1), (A3, B2, C2, D3, E1), (A4, B2, C2, D3, E1), (A5, B2, C2, D3, E1),
(A1, B3, C2, D3, E1), (A2, B3, C2, D3, E1), (A3, B3, C2, D3, E1), (A4, B3, C2, D3, E1), (A5, B3, C2, D3, E1),
(A1, B4, C2, D3, E1), (A2, B4, C2, D3, E1), (A3, B4, C2, D3, E1), (A4, B4, C2, D3, E1), (A5, B4, C2, D3, E1),
(A1, B1, C1, D4, E1), (A2, B1, C1, D4, E1), (A3, B1, C1, D4, E1), (A4, B1, C1, D4, E1), (A5, B1, C1, D4, E1),
(A1, B2, C1, D4, E1), (A2, B2, C1, D4, E1), (A3, B2, C1, D4, E1), (A4, B2, C1, D4, E1), (A5, B2, C1, D4, E1),
(A1, B3, C1, D4, E1), (A2, B3, C1, D4, E1), (A3, B3, C1, D4, E1), (A4, B3, C1, D4, E1), (A5, B3, C1, D4, E1),
(A1, B4, C1, D4, E1), (A2, B4, C1, D4, E1), (A3, B4, C1, D4, E1), (A4, B4, C1, D4, E1), (A5, B4, C1, D4, E1),
(A1, B1, C2, D4, E1), (A2, B1, C2, D4, E1), (A3, B1, C2, D4, E1), (A4, B1, C2, D4, E1), (A5, B1, C2, D4, E1),
(A1, B2, C2, D4, E1), (A2, B2, C2, D4, E1), (A3, B2, C2, D4, E1), (A4, B2, C2, D4, E1), (A5, B2, C2, D4, E1),
(A1, B3, C2, D4, E1), (A2, B3, C2, D4, E1), (A3, B3, C2, D4, E1), (A4, B3, C2, D4, E1), (A5, B3, C2, D4, E1),
(A1, B4, C2, D4, E1), (A2, B4, C2, D4, E1), (A3, B4, C2, D4, E1), (A4, B4, C2, D4, E1), (A5, B4, C2, D4, E1),
(A1, B1, C1, D5, E1), (A2, B1, C1, D5, E1), (A3, B1, C1, D5, E1), (A4, B1, C1, D5, E1), (A5, B1, C1, D5, E1),
(A1, B2, C1, D5, E1), (A2, B2, C1, D5, E1), (A3, B2, C1, D5, E1), (A4, B2, C1, D5, E1), (A5, B2, C1, D5, E1),
(A1, B3, C1, D5, E1), (A2, B3, C1, D5, E1), (A3, B3, C1, D5, E1), (A4, B3, C1, D5, E1), (A5, B3, C1, D5, E1),
(A1, B4, C1, D5, E1), (A2, B4, C1, D5, E1), (A3, B4, C1, D5, E1), (A4, B4, C1, D5, E1), (A5, B4, C1, D5, E1),
(A1, B1, C2, D5, E1), (A2, B1, C2, D5, E1), (A3, B1, C2, D5, E1), (A4, B1, C2, D5, E1), (A5, B1, C2, D5, E1),
(A1, B2, C2, D5, E1), (A2, B2, C2, D5, E1), (A3, B2, C2, D5, E1), (A4, B2, C2, D5, E1), (A5, B2, C2, D5, E1),
(A1, B3, C2, D5, E1), (A2, B3, C2, D5, E1), (A3, B3, C2, D5, E1), (A4, B3, C2, D5, E1), (A5, B3, C2, D5, E1),
(A1, B4, C2, D5, E1), (A2, B4, C2, D5, E1), (A3, B4, C2, D5, E1), (A4, B4, C2, D5, E1), (A5, B4, C2, D5, E1),
(A1, B1, C1, D1, E2), (A2, B1, C1, D1, E2), (A3, B1, C1, D1, E2), (A4, B1, C1, D1, E2), (A5, B1, C1, D1, E2),
(A1, B2, C1, D1, E2), (A2, B2, C1, D1, E2), (A3, B2, C1, D1, E2), (A4, B2, C1, D1, E2), (A5, B2, C1, D1, E2),
(A1, B3, C1, D1, E2), (A2, B3, C1, D1, E2), (A3, B3, C1, D1, E2), (A4, B3, C1, D1, E2), (A5, B3, C1, D1, E2),
(A1, B4, C1, D1, E2), (A2, B4, C1, D1, E2), (A3, B4, C1, D1, E2), (A4, B4, C1, D1, E2), (A5, B4, C1, D1, E2),
(A1, B1, C2, D1, E2), (A2, B1, C2, D1, E2), (A3, B1, C2, D1, E2), (A4, B1, C2, D1, E2), (A5, B1, C2, D1, E2),
(A1, B2, C2, D1, E2), (A2, B2, C2, D1, E2), (A3, B2, C2, D1, E2), (A4, B2, C2, D1, E2), (A5, B2, C2, D1, E2),
(A1, B3, C2, D1, E2), (A2, B3, C2, D1, E2), (A3, B3, C2, D1, E2), (A4, B3, C2, D1, E2), (A5, B3, C2, D1, E2),
(A1, B4, C2, D1, E2), (A2, B4, C2, D1, E2), (A3, B4, C2, D1, E2), (A4, B4, C2, D1, E2), (A5, B4, C2, D1, E2),
(A1, B1, C1, D2, E2), (A2, B1, C1, D2, E2), (A3, B1, C1, D2, E2), (A4, B1, C1, D2, E2), (A5, B1, C1, D2, E2),
(A1, B2, C1, D2, E2), (A2, B2, C1, D2, E2), (A3, B2, C1, D2, E2), (A4, B2, C1, D2, E2), (A5, B2, C1, D2, E2),
(A1, B3, C1, D2, E2), (A2, B3, C1, D2, E2), (A3, B3, C1, D2, E2), (A4, B3, C1, D2, E2), (A5, B3, C1, D2, E2),
(A1, B4, C1, D2, E2), (A2, B4, C1, D2, E2), (A3, B4, C1, D2, E2), (A4, B4, C1, D2, E2), (A5, B4, C1, D2, E2),
(A1, B1, C2, D2, E2), (A2, B1, C2, D2, E2), (A3, B1, C2, D2, E2), (A4, B1, C2, D2, E2), (A5, B1, C2, D2, E2),
(A1, B2, C2, D2, E2), (A2, B2, C2, D2, E2), (A3, B2, C2, D2, E2), (A4, B2, C2, D2, E2), (A5, B2, C2, D2, E2),
(A1, B3, C2, D2, E2), (A2, B3, C2, D2, E2), (A3, B3, C2, D2, E2), (A4, B3, C2, D2, E2), (A5, B3, C2, D2, E2),
(A1, B4, C2, D2, E2), (A2, B4, C2, D2, E2), (A3, B4, C2, D2, E2), (A4, B4, C2, D2, E2), (A5, B4, C2, D2, E2),
(A1, B1, C1, D3, E2), (A2, B1, C1, D3, E2), (A3, B1, C1, D3, E2), (A4, B1, C1, D3, E2), (A5, B1, C1, D3, E2),
(A1, B2, C1, D3, E2), (A2, B2, C1, D3, E2), (A3, B2, C1, D3, E2), (A4, B2, C1, D3, E2), (A5, B2, C1, D3, E2),
(A1, B3, C1, D3, E2), (A2, B3, C1, D3, E2), (A3, B3, C1, D3, E2), (A4, B3, C1, D3, E2), (A5, B3, C1, D3, E2),
(A1, B4, C1, D3, E2), (A2, B4, C1, D3, E2), (A3, B4, C1, D3, E2), (A4, B4, C1, D3, E2), (A5, B4, C1, D3, E2),
(A1, B1, C2, D3, E2), (A2, B1, C2, D3, E2), (A3, B1, C2, D3, E2), (A4, B1, C2, D3, E2), (A5, B1, C2, D3, E2),
(A1, B2, C2, D3, E2), (A2, B2, C2, D3, E2), (A3, B2, C2, D3, E2), (A4, B2, C2, D3, E2), (A5, B2, C2, D3, E2),
(A1, B3, C2, D3, E2), (A2, B3, C2, D3, E2), (A3, B3, C2, D3, E2), (A4, B3, C2, D3, E2), (A5, B3, C2, D3, E2),
(A1, B4, C2, D3, E2), (A2, B4, C2, D3, E2), (A3, B4, C2, D3, E2), (A4, B4, C2, D3, E2), (A5, B4, C2, D3, E2),
(A1, B1, C1, D4, E2), (A2, B1, C1, D4, E2), (A3, B1, C1, D4, E2), (A4, B1, C1, D4, E2), (A5, B1, C1, D4, E2),
(A1, B2, C1, D4, E2), (A2, B2, C1, D4, E2), (A3, B2, C1, D4, E2), (A4, B2, C1, D4, E2), (A5, B2, C1, D4, E2),
(A1, B3, C1, D4, E2), (A2, B3, C1, D4, E2), (A3, B3, C1, D4, E2), (A4, B3, C1, D4, E2), (A5, B3, C1, D4, E2),
(A1, B4, C1, D4, E2), (A2, B4, C1, D4, E2), (A3, B4, C1, D4, E2), (A4, B4, C1, D4, E2), (A5, B4, C1, D4, E2),
(A1, B1, C2, D4, E2), (A2, B1, C2, D4, E2), (A3, B1, C2, D4, E2), (A4, B1, C2, D4, E2), (A5, B1, C2, D4, E2),
(A1, B2, C2, D4, E2), (A2, B2, C2, D4, E2), (A3, B2, C2, D4, E2), (A4, B2, C2, D4, E2), (A5, B2, C2, D4, E2),
(A1, B3, C2, D4, E2), (A2, B3, C2, D4, E2), (A3, B3, C2, D4, E2), (A4, B3, C2, D4, E2), (A5, B3, C2, D4, E2),
(A1, B4, C2, D4, E2), (A2, B4, C2, D4, E2), (A3, B4, C2, D4, E2), (A4, B4, C2, D4, E2), (A5, B4, C2, D4, E2),
(A1, B1, C1, D5, E2), (A2, B1, C1, D5, E2), (A3, B1, C1, D5, E2), (A4, B1, C1, D5, E2), (A5, B1, C1, D5, E2),
(A1, B2, C1, D5, E2), (A2, B2, C1, D5, E2), (A3, B2, C1, D5, E2), (A4, B2, C1, D5, E2), (A5, B2, C1, D5, E 2),
(A1, B3, C1, D5, E2), (A2, B3, C1, D5, E2), (A3, B3, C1, D5, E2), (A4, B3, C1, D5, E2), (A5, B3, C1, D5, E2),
(A1, B4, C1, D5, E2), (A2, B4, C1, D5, E2), (A3, B4, C1, D5, E2), (A4, B4, C1, D5, E2), (A5, B4, C1, D5, E2),
(A1, B1, C2, D5, E2), (A2, B1, C2, D5, E2), (A3, B1, C2, D5, E2), (A4, B1, C2, D5, E2), (A5, B1, C2, D5, E2),
(A1, B2, C2, D5, E2), (A2, B2, C2, D5, E2), (A3, B2, C2, D5, E2), (A4, B2, C2, D5, E2), (A5, B2, C2, D5, E2),
(A1, B3, C2, D5, E2), (A2, B3, C2, D5, E2), (A3, B3, C2, D5, E2), (A4, B3, C2, D5, E2), (A5, B3, C2, D5, E2),
(A1, B4, C2, D5, E2), (A2, B4, C2, D5, E2), (A3, B4, C2, D5, E2), (A4, B4, C2, D5, E2), (A5, B4, C2, D5, E2),
(A1, B1, C1, D1, E3), (A2, B1, C1, D1, E3), (A3, B1, C1, D1, E3), (A4, B1, C1, D1, E3), (A5, B1, C1, D1, E3),
(A1, B2, C1, D1, E3), (A2, B2, C1, D1, E3), (A3, B2, C1, D1, E3), (A4, B2, C1, D1, E3), (A5, B2, C1, D1, E3),
(A1, B3, C1, D1, E3), (A2, B3, C1, D1, E3), (A3, B3, C1, D1, E3), (A4, B3, C1, D1, E3), (A5, B3, C1, D1, E3),
(A1, B4, C1, D1, E3), (A2, B4, C1, D1, E3), (A3, B4, C1, D1, E3), (A4, B4, C1, D1, E3), (A5, B4, C1, D1, E3),
(A1, B1, C2, D1, E3), (A2, B1, C2, D1, E3), (A3, B1, C2, D1, E3), (A4, B1, C2, D1, E3), (A5, B1, C2, D1, E3),
(A1, B2, C2, D1, E3), (A2, B2, C2, D1, E3), (A3, B2, C2, D1, E3), (A4, B2, C2, D1, E3), (A5, B2, C2, D1, E3),
(A1, B3, C2, D1, E3), (A2, B3, C2, D1, E3), (A3, B3, C2, D1, E3), (A4, B3, C2, D1, E3), (A5, B3, C2, D1, E3),
(A1, B4, C2, D1, E3), (A2, B4, C2, D1, E3), (A3, B4, C2, D1, E3), (A4, B4, C2, D1, E3), (A5, B4, C2, D1, E3),
(A1, B1, C1, D2, E3), (A2, B1, C1, D2, E3), (A3, B1, C1, D2, E3), (A4, B1, C1, D2, E3), (A5, B1, C1, D2, E3),
(A1, B2, C1, D2, E3), (A2, B2, C1, D2, E3), (A3, B2, C1, D2, E3), (A4, B2, C1, D2, E3), (A5, B2, C1, D2, E3),
(A1, B3, C1, D2, E3), (A2, B3, C1, D2, E3), (A3, B3, C1, D2, E3), (A4, B3, C1, D2, E3), (A5, B3, C1, D2, E3),
(A1, B4, C1, D2, E3), (A2, B4, C1, D2, E3), (A3, B4, C1, D2, E3), (A4, B4, C1, D2, E3), (A5, B4, C1, D2, E3),
(A1, B1, C2, D2, E3), (A2, B1, C2, D2, E3), (A3, B1, C2, D2, E3), (A4, B1, C2, D2, E3), (A5, B1, C2, D2, E3),
(A1, B2, C2, D2, E3), (A2, B2, C2, D2, E3), (A3, B2, C2, D2, E3), (A4, B2, C2, D2, E3), (A5, B2, C2, D2, E3),
(A1, B3, C2, D2, E3), (A2, B3, C2, D2, E3), (A3, B3, C2, D2, E3), (A4, B3, C2, D2, E3), (A5, B3, C2, D2, E3),
(A1, B4, C2, D2, E3), (A2, B4, C2, D2, E3), (A3, B4, C2, D2, E3), (A4, B4, C2, D2, E3), (A5, B4, C2, D2, E3),
(A1, B1, C1, D3, E3), (A2, B1, C1, D3, E3), (A3, B1, C1, D3, E3), (A4, B1, C1, D3, E3), (A5, B1, C1, D3, E3),
(A1, B2, C1, D3, E3), (A2, B2, C1, D3, E3), (A3, B2, C1, D3, E3), (A4, B2, C1, D3, E3), (A5, B2, C1, D3, E3),
(A1, B3, C1, D3, E3), (A2, B3, C1, D3, E3), (A3, B3, C1, D3, E3), (A4, B3, C1, D3, E3), (A5, B3, C1, D3, E3),
(A1, B4, C1, D3, E3), (A2, B4, C1, D3, E3), (A3, B4, C1, D3, E3), (A4, B4, C1, D3, E3), (A5, B4, C1, D3, E3),
(A1, B1, C2, D3, E3), (A2, B1, C2, D3, E3), (A3, B1, C2, D3, E3), (A4, B1, C2, D3, E3), (A5, B1, C2, D3, E3),
(A1, B2, C2, D3, E3), (A2, B2, C2, D3, E3), (A3, B2, C2, D3, E3), (A4, B2, C2, D3, E3), (A5, B2, C2, D3, E3),
(A1, B3, C2, D3, E3), (A2, B3, C2, D3, E3), (A3, B3, C2, D3, E3), (A4, B3, C2, D3, E3), (A5, B3, C2, D3, E3),
(A1, B4, C2, D3, E3), (A2, B4, C2, D3, E3), (A3, B4, C2, D3, E3), (A4, B4, C2, D3, E3), (A5, B4, C2, D3, E3),
(A1, B1, C1, D4, E3), (A2, B1, C1, D4, E3), (A3, B1, C1, D4, E3), (A4, B1, C1, D4, E3), (A5, B1, C1, D4, E3),
(A1, B2, C1, D4, E3), (A2, B2, C1, D4, E3), (A3, B2, C1, D4, E3), (A4, B2, C1, D4, E3), (A5, B2, C1, D4, E3),
(A1, B3, C1, D4, E3), (A2, B3, C1, D4, E3), (A3, B3, C1, D4, E3), (A4, B3, C1, D4, E3), (A5, B3, C1, D4, E3),
(A1, B4, C1, D4, E3), (A2, B4, C1, D4, E3), (A3, B4, C1, D4, E3), (A4, B4, C1, D4, E3), (A5, B4, C1, D4, E3),
(A1, B1, C2, D4, E3), (A2, B1, C2, D4, E3), (A3, B1, C2, D4, E3), (A4, B1, C2, D4, E3), (A5, B1, C2, D4, E3),
(A1, B2, C2, D4, E3), (A2, B2, C2, D4, E3), (A3, B2, C2, D4, E3), (A4, B2, C2, D4, E3), (A5, B2, C2, D4, E3),
(A1, B3, C2, D4, E3), (A2, B3, C2, D4, E3), (A3, B3, C2, D4, E3), (A4, B3, C2, D4, E3), (A5, B3, C2, D4, E3),
(A1, B4, C2, D4, E3), (A2, B4, C2, D4, E3), (A3, B4, C2, D4, E3), (A4, B4, C2, D4, E3), (A5, B4, C2, D4, E3),
(A1, B1, C1, D5, E3), (A2, B1, C1, D5, E3), (A3, B1, C1, D5, E3), (A4, B1, C1, D5, E3), (A5, B1, C1, D5, E3),
(A1, B2, C1, D5, E3), (A2, B2, C1, D5, E3), (A3, B2, C1, D5, E3), (A4, B2, C1, D5, E3), (A5, B2, C1, D5, E3),
(A1, B3, C1, D5, E3), (A2, B3, C1, D5, E3), (A3, B3, C1, D5, E3), (A4, B3, C1, D5, E3), (A5, B3, C1, D5, E3),
(A1, B4, C1, D5, E3), (A2, B4, C1, D5, E3), (A3, B4, C1, D5, E3), (A4, B4, C1, D5, E3), (A5, B4, C1, D5, E3),
(A1, B1, C2, D5, E3), (A2, B1, C2, D5, E3), (A3, B1, C2, D5, E3), (A4, B1, C2, D5, E3), (A5, B1, C2, D5, E3),
(A1, B2, C2, D5, E3), (A2, B2, C2, D5, E3), (A3, B2, C2, D5, E3), (A4, B2, C2, D5, E3), (A5, B2, C2, D5, E3),
(A1, B3, C2, D5, E3), (A2, B3, C2, D5, E3), (A3, B3, C2, D5, E3), (A4, B3, C2, D5, E3), (A5, B3, C2, D5, E3),
(A1, B4, C2, D5, E3), (A2, B4, C2, D5, E3), (A3, B4, C2, D5, E3), (A4, B4, C2, D5, E3), (A5, B4, C2, D5, E3),
(A1, B1, C1, D1, E4), (A2, B1, C1, D1, E4), (A3, B1, C1, D1, E4), (A4, B1, C1, D1, E4), (A5, B1, C1, D1, E4),
(A1, B2, C1, D1, E4), (A2, B2, C1, D1, E4), (A3, B2, C1, D1, E4), (A4, B2, C1, D1, E4), (A5, B2, C1, D1, E4),
(A1, B3, C1, D1, E4), (A2, B3, C1, D1, E4), (A3, B3, C1, D1, E4), (A4, B3, C1, D1, E4), (A5, B3, C1, D1, E4),
(A1, B4, C1, D1, E4), (A2, B4, C1, D1, E4), (A3, B4, C1, D1, E4), (A4, B4, C1, D1, E4), (A5, B4, C1, D1, E4),
(A1, B1, C2, D1, E4), (A2, B1, C2, D1, E4), (A3, B1, C2, D1, E4), (A4, B1, C2, D1, E4), (A5, B1, C2, D1, E4),
(A1, B2, C2, D1, E4), (A2, B2, C2, D1, E4), (A3, B2, C2, D1, E4), (A4, B2, C2, D1, E4), (A5, B2, C2, D1, E4),
(A1, B3, C2, D1, E4), (A2, B3, C2, D1, E4), (A3, B3, C2, D1, E4), (A4, B3, C2, D1, E4), (A5, B3, C2, D1, E4),
(A1, B4, C2, D1, E4), (A2, B4, C2, D1, E4), (A3, B4, C2, D1, E4), (A4, B4, C2, D1, E4), (A5, B4, C2, D1, E4),
(A1, B1, C1, D2, E4), (A2, B1, C1, D2, E4), (A3, B1, C1, D2, E4), (A4, B1, C1, D2, E4), (A5, B1, C1, D2, E4),
(A1, B2, C1, D2, E4), (A2, B2, C1, D2, E4), (A3, B2, C1, D2, E4), (A4, B2, C1, D2, E4), (A5, B2, C1, D2, E4),
(A1, B3, C1, D2, E4), (A2, B3, C1, D2, E4), (A3, B3, C1, D2, E4), (A4, B3, C1, D2, E4), (A5, B3, C1, D2, E4),
(A1, B4, C1, D2, E4), (A2, B4, C1, D2, E4), (A3, B4, C1, D2, E4), (A4, B4, C1, D2, E4), (A5, B4, C1, D2, E4),
(A1, B1, C2, D2, E4), (A2, B1, C2, D2, E4), (A3, B1, C2, D2, E4), (A4, B1, C2, D2, E4), (A5, B1, C2, D2, E4),
(A1, B2, C2, D2, E4), (A2, B2, C2, D2, E4), (A3, B2, C2, D2, E4), (A4, B2, C2, D2, E4), (A5, B2, C2, D2, E4),
(A1, B3, C2, D2, E4), (A2, B3, C2, D2, E4), (A3, B3, C2, D2, E4), (A4, B3, C2, D2, E4), (A5, B3, C2, D2, E4),
(A1, B4, C2, D2, E4), (A2, B4, C2, D2, E4), (A3, B4, C2, D2, E4), (A4, B4, C2, D2, E4), (A5, B4, C2, D2, E4),
(A1, B1, C1, D3, E4), (A2, B1, C1, D3, E4), (A3, B1, C1, D3, E4), (A4, B1, C1, D3, E4), (A5, B1, C1, D3, E4),
(A1, B2, C1, D3, E4), (A2, B2, C1, D3, E4), (A3, B2, C1, D3, E4), (A4, B2, C1, D3, E4), (A5, B2, C1, D3, E4),
(A1, B3, C1, D3, E4), (A2, B3, C1, D3, E4), (A3, B3, C1, D3, E4), (A4, B3, C1, D3, E4), (A5, B3, C1, D3, E4),
(A1, B4, C1, D3, E4), (A2, B4, C1, D3, E4), (A3, B4, C1, D3, E4), (A4, B4, C1, D3, E4), (A5, B4, C1, D3, E4),
(A1, B1, C2, D3, E4), (A2, B1, C2, D3, E4), (A3, B1, C2, D3, E4), (A4, B1, C2, D3, E4), (A5, B1, C2, D3, E4),
(A1, B2, C2, D3, E4), (A2, B2, C2, D3, E4), (A3, B2, C2, D3, E4), (A4, B2, C2, D3, E4), (A5, B2, C2, D3, E4),
(A1, B3, C2, D3, E4), (A2, B3, C2, D3, E4), (A3, B3, C2, D3, E4), (A4, B3, C2, D3, E4), (A5, B3, C2, D3, E4),
(A1, B4, C2, D3, E4), (A2, B4, C2, D3, E4), (A3, B4, C2, D3, E4), (A4, B4, C2, D3, E4), (A5, B4, C2, D3, E4),
(A1, B1, C1, D4, E4), (A2, B1, C1, D4, E4), (A3, B1, C1, D4, E4), (A4, B1, C1, D4, E4), (A5, B1, C1, D4, E4),
(A1, B2, C1, D4, E4), (A2, B2, C1, D4, E4), (A3, B2, C1, D4, E4), (A4, B2, C1, D4, E4), (A5, B2, C1, D4, E4),
(A1, B3, C1, D4, E4), (A2, B3, C1, D4, E4), (A3, B3, C1, D4, E4), (A4, B3, C1, D4, E4), (A5, B3, C1, D4, E4),
(A1, B4, C1, D4, E4), (A2, B4, C1, D4, E4), (A3, B4, C1, D4, E4), (A4, B4, C1, D4, E4), (A5, B4, C1, D4, E4),
(A1, B1, C2, D4, E4), (A2, B1, C2, D4, E4), (A3, B1, C2, D4, E4), (A4, B1, C2, D4, E4), (A5, B1, C2, D4, E4),
(A1, B2, C2, D4, E4), (A2, B2, C2, D4, E4), (A3, B2, C2, D4, E4), (A4, B2, C2, D4, E4), (A5, B2, C2, D4, E4),
(A1, B3, C2, D4, E4), (A2, B3, C2, D4, E4), (A3, B3, C2, D4, E4), (A4, B3, C2, D4, E4), (A5, B3, C2, D4, E4),
(A1, B4, C2, D4, E4), (A2, B4, C2, D4, E4), (A3, B4, C2, D4, E4), (A4, B4, C2, D4, E4), (A5, B4, C2, D4, E4),
(A1, B1, C1, D5, E4), (A2, B1, C1, D5, E4), (A3, B1, C1, D5, E4), (A4, B1, C1, D5, E4), (A5, B1, C1, D5, E4),
(A1, B2, C1, D5, E4), (A2, B2, C1, D5, E4), (A3, B2, C1, D5, E4), (A4, B2, C1, D5, E4), (A5, B2, C1, D5, E4),
(A1, B3, C1, D5, E4), (A2, B3, C1, D5, E4), (A3, B3, C1, D5, E4), (A4, B3, C1, D5, E4), (A5, B3, C1, D5, E4),
(A1, B4, C1, D5, E4), (A2, B4, C1, D5, E4), (A3, B4, C1, D5, E4), (A4, B4, C1, D5, E4), (A5, B4, C1, D5, E4),
(A1, B1, C2, D5, E4), (A2, B1, C2, D5, E4), (A3, B1, C2, D5, E4), (A4, B1, C2, D5, E4), (A5, B1, C2, D5, E4),
(A1, B2, C2, D5, E4), (A2, B2, C2, D5, E4), (A3, B2, C2, D5, E4), (A4, B2, C2, D5, E4), (A5, B2, C2, D5, E4),
(A1, B3, C2, D5, E4), (A2, B3, C2, D5, E4), (A3, B3, C2, D5, E4), (A4, B3, C2, D5, E4), (A5, B3, C2, D5, E4),
(A1, B4, C2, D5, E4), (A2, B4, C2, D5, E4), (A3, B4, C2, D5, E4), (A4, B4, C2, D5, E4), (A5, B4, C2, D5, E4),
(A1, B1, C1, D1, E5), (A2, B1, C1, D1, E5), (A3, B1, C1, D1, E5), (A4, B1, C1, D1, E5), (A5, B1, C1, D1, E5),
(A1, B2, C1, D1, E5), (A2, B2, C1, D1, E5), (A3, B2, C1, D1, E5), (A4, B2, C1, D1, E5), (A5, B2, C1, D1, E5),
(A1, B3, C1, D1, E5), (A2, B3, C1, D1, E5), (A3, B3, C1, D1, E5), (A4, B3, C1, D1, E5), (A5, B3, C1, D1, E5),
(A1, B4, C1, D1, E5), (A2, B4, C1, D1, E5), (A3, B4, C1, D1, E5), (A4, B4, C1, D1, E5), (A5, B4, C1, D1, E5),
(A1, B1, C2, D1, E5), (A2, B1, C2, D1, E5), (A3, B1, C2, D1, E5), (A4, B1, C2, D1, E5), (A5, B1, C2, D1, E5),
(A1, B2, C2, D1, E5), (A2, B2, C2, D1, E5), (A3, B2, C2, D1, E5), (A4, B2, C2, D1, E5), (A5, B2, C2, D1, E5),
(A1, B3, C2, D1, E5), (A2, B3, C2, D1, E5), (A3, B3, C2, D1, E5), (A4, B3, C2, D1, E5), (A5, B3, C2, D1, E5),
(A1, B4, C2, D1, E5), (A2, B4, C2, D1, E5), (A3, B4, C2, D1, E5), (A4, B4, C2, D1, E5), (A5, B4, C2, D1, E5),
(A1, B1, C1, D2, E5), (A2, B1, C1, D2, E5), (A3, B1, C1, D2, E5), (A4, B1, C1, D2, E5), (A5, B1, C1, D2, E5),
(A1, B2, C1, D2, E5), (A2, B2, C1, D2, E5), (A3, B2, C1, D2, E5), (A4, B2, C1, D2, E5), (A5, B2, C1, D2, E5),
(A1, B3, C1, D2, E5), (A2, B3, C1, D2, E5), (A3, B3, C1, D2, E5), (A4, B3, C1, D2, E5), (A5, B3, C1, D2, E5),
(A1, B4, C1, D2, E5), (A2, B4, C1, D2, E5), (A3, B4, C1, D2, E5), (A4, B4, C1, D2, E5), (A5, B4, C1, D2, E5),
(A1, B1, C2, D2, E5), (A2, B1, C2, D2, E5), (A3, B1, C2, D2, E5), (A4, B1, C2, D2, E5), (A5, B1, C2, D2, E5),
(A1, B2, C2, D2, E5), (A2, B2, C2, D2, E5), (A3, B2, C2, D2, E5), (A4, B2, C2, D2, E5), (A5, B2, C2, D2, E5),
(A1, B3, C2, D2, E5), (A2, B3, C2, D2, E5), (A3, B3, C2, D2, E5), (A4, B3, C2, D2, E5), (A5, B3, C2, D2, E5),
(A1, B4, C2, D2, E5), (A2, B4, C2, D2, E5), (A3, B4, C2, D2, E5), (A4, B4, C2, D2, E5), (A5, B4, C2, D2, E5),
(A1, B1, C1, D3, E5), (A2, B1, C1, D3, E5), (A3, B1, C1, D3, E5), (A4, B1, C1, D3, E5), (A5, B1, C1, D3, E5),
(A1, B2, C1, D3, E5), (A2, B2, C1, D3, E5), (A3, B2, C1, D3, E5), (A4, B2, C1, D3, E5), (A5, B2, C1, D3, E5),
(A1, B3, C1, D3, E5), (A2, B3, C1, D3, E5), (A3, B3, C1, D3, E5), (A4, B3, C1, D3, E5), (A5, B3, C1, D3, E5),
(A1, B4, C1, D3, E5), (A2, B4, C1, D3, E5), (A3, B4, C1, D3, E5), (A4, B4, C1, D3, E5), (A5, B4, C1, D3, E5),
(A1, B1, C2, D3, E5), (A2, B1, C2, D3, E5), (A3, B1, C2, D3, E5), (A4, B1, C2, D3, E5), (A5, B1, C2, D3, E5),
(A1, B2, C2, D3, E5), (A2, B2, C2, D3, E5), (A3, B2, C2, D3, E5), (A4, B2, C2, D3, E5), (A5, B2, C2, D3, E5),
(A1, B3, C2, D3, E5), (A2, B3, C2, D3, E5), (A3, B3, C2, D3, E5), (A4, B3, C2, D3, E5), (A5, B3, C2, D3, E5),
(A1, B4, C2, D3, E5), (A2, B4, C2, D3, E5), (A3, B4, C2, D3, E5), (A4, B4, C2, D3, E5), (A5, B4, C2, D3, E5),
(A1, B1, C1, D4, E5), (A2, B1, C1, D4, E5), (A3, B1, C1, D4, E5), (A4, B1, C1, D4, E5), (A5, B1, C1, D4, E5),
(A1, B2, C1, D4, E5), (A2, B2, C1, D4, E5), (A3, B2, C1, D4, E5), (A4, B2, C1, D4, E5), (A5, B2, C1, D4, E5),
(A1, B3, C1, D4, E5), (A2, B3, C1, D4, E5), (A3, B3, C1, D4, E5), (A4, B3, C1, D4, E5), (A5, B3, C1, D4, E5),
(A1, B4, C1, D4, E5), (A2, B4, C1, D4, E5), (A3, B4, C1, D4, E5), (A4, B4, C1, D4, E5), (A5, B4, C1, D4, E5),
(A1, B1, C2, D4, E5), (A2, B1, C2, D4, E5), (A3, B1, C2, D4, E5), (A4, B1, C2, D4, E5), (A5, B1, C2, D4, E5),
(A1, B2, C2, D4, E5), (A2, B2, C2, D4, E5), (A3, B2, C2, D4, E5), (A4, B2, C2, D4, E5), (A5, B2, C2, D4, E5),
(A1, B3, C2, D4, E5), (A2, B3, C2, D4, E5), (A3, B3, C2, D4, E5), (A4, B3, C2, D4, E5), (A5, B3, C2, D4, E5),
(A1, B4, C2, D4, E5), (A2, B4, C2, D4, E5), (A3, B4, C2, D4, E5), (A4, B4, C2, D4, E5), (A5, B4, C2, D4, E5),
(A1, B1, C1, D5, E5), (A2, B1, C1, D5, E5), (A3, B1, C1, D5, E5), (A4, B1, C1, D5, E5), (A5, B1, C1, D5, E5),
(A1, B2, C1, D5, E5), (A2, B2, C1, D5, E5), (A3, B2, C1, D5, E5), (A4, B2, C1, D5, E5), (A5, B2, C1, D5, E5),
(A1, B3, C1, D5, E5), (A2, B3, C1, D5, E5), (A3, B3, C1, D5, E5), (A4, B3, C1, D5, E5), (A5, B3, C1, D5, E5),
(A1, B4, C1, D5, E5), (A2, B4, C1, D5, E5), (A3, B4, C1, D5, E5), (A4, B4, C1, D5, E5), (A5, B4, C1, D5, E5),
(A1, B1, C2, D5, E5), (A2, B1, C2, D5, E5), (A3, B1, C2, D5, E5), (A4, B1, C2, D5, E5), (A5, B1, C2, D5, E5),
(A1, B2, C2, D5, E5), (A2, B2, C2, D5, E5), (A3, B2, C2, D5, E5), (A4, B2, C2, D5, E5), (A5, B2, C2, D5, E5),
(A1, B3, C2, D5, E5), (A2, B3, C2, D5, E5), (A3, B3, C2, D5, E5), (A4, B3, C2, D5, E5), (A5, B3, C2, D5, E5),
(A1, B4, C2, D5, E5), (A2, B4, C2, D5, E5), (A3, B4, C2, D5, E5), (A4, B4, C2, D5, E5), and/or (A5, B4, C2, D5,
E5).

TABLE 6
Multiplexing of the Primer/Probe Combinations shown in Table 3 for detection
of S. aureus, H. influenzae, M. catarrhalis, and K. pneumoniae

(F1, G1, H1, I1), (F2, G1, H1, I1), (F3, G1, H1, I1), (F4, G1, H1, I1), (F5, G1, H1, I1), (F1, G2, H1, I1), (F2, G2,
H1, I1), (F3, G2, H1, I1), (F4, G2, H1, I1), (F5, G2, H1, I1), (F1, G3, H1, I1), (F2, G3, H1, I1), (F3, G3, H1, I1),
(F4, G3, H1, I1), (F5, G3, H1, I1), (F1, G4, H1, I1), (F2, G4, H1, I1), (F3, G4, H1, I1), (F4, G4, H1, I1), (F5, G4,
H1, I1), (F1, G1, H2, I1), (F2, G1, H2, I1), (F3, G1, H2, I1), (F4, G1, H2, I1), (F5, G1, H2, I1), (F1, G2, H2, I1),
(F2, G2, H2, I1), (F3, G2, H2, I1), (F4, G2, H2, I1), (F5, G2, H2, I1), (F1, G3, H2, I1), (F2, G3, H2, I1), (F3, G3,
H2, I1), (F4, G3, H2, I1), (F5, G3, H2, I1), (F1, G4, H2, I1), (F2, G4, H2, I1), (F3, G4, H2, I1), (F4, G4, H2, I1),
(F5, G4, H2, I1), (F1, G1, H1, I2), (F2, G1, H1, I2), (F3, G1, H1, I2), (F4, G1, H1, I2), (F5, G1, H1, I2), (F1, G2,
H1, I2), (F2, G2, H1, I2), (F3, G2, H1, I2), (F4, G2, H1, I2), (F5, G2, H1, I2), (F1, G3, H1, I2), (F2, G3, H1, I2),
(F3, G3, H1, I2), (F4, G3, H1, I2), (F5, G3, H1, I2), (F1, G4, H1, I2), (F2, G4, H1, I2), (F3, G4, H1, I2), (F4, G4,
H1, I2), (F5, G4, H1, I2), (F1, G1, H2, I2), (F2, G1, H2, I2), (F3, G1, H2, I2), (F4, G1, H2, I2), (F5, G1, H2, I2),
(F1, G2, H2, I2), (F2, G2, H2, I2), (F3, G2, H2, I2), (F4, G2, H2, I2), (F5, G2, H2, I2), (F1, G3, H2, I2), (F2, G3,
H2, I2), (F3, G3, H2, I2), (F4, G3, H2, I2), (F5, G3, H2, I2), (F1, G4, H2, I2), (F2, G4, H2, I2), (F3, G4, H2, I2),
(F4, G4, H2, I2), (F5, G4, H2, I2), (F1, G1, H1, I3), (F2, G1, H1, I3), (F3, G1, H1, I3), (F4, G1, H1, I3), (F5, G1,
H1, I3), (F1, G2, H1, I3), (F2, G2, H1, I3), (F3, G2, H1, I3), (F4, G2, H1, I3), (F5, G2, H1, I3), (F1, G3, H1, I3),
(F2, G3, H1, I3), (F3, G3, H1, I3), (F4, G3, H1, I3), (F5, G3, H1, I3), (F1, G4, H1, I3), (F2, G4, H1, I3), (F3, G4,
H1, I3), (F4, G4, H1, I3), (F5, G4, H1, I3), (F1, G1, H2, I3), (F2, G1, H2, I3), (F3, G1, H2, I3), (F4, G1, H2, I3),
(F5, G1, H2, I3), (F1, G2, H2, I3), (F2, G2, H2, I3), (F3, G2, H2, I3), (F4, G2, H2, I3), (F5, G2, H2, I3), (F1, G3,
H2, I3), (F2, G3, H2, I3), (F3, G3, H2, I3), (F4, G3, H2, I3), (F5, G3, H2, I3), (F1, G4, H2, I3), (F2, G4, H2, I3),
(F3, G4, H2, I3), (F4, G4, H2, I3), (F5, G4, H2, I3), (F1, G1, H1, I4), (F2, G1, H1, I4), (F3, G1, H1, I4), (F4, G1,
H1, I4), (F5, G1, H1, I4), (F1, G2, H1, I4), (F2, G2, H1, I4), (F3, G2, H1, I4), (F4, G2, H1, I4), (F5, G2, H1, I4),
(F1, G3, H1, I4), (F2, G3, H1, I4), (F3, G3, H1, I4), (F4, G3, H1, I4), (F5, G3, H1, I4), (F1, G4, H1, I4), (F2, G4,
H1, I4), (F3, G4, H1, I4), (F4, G4, H1, I4), (F5, G4, H1, I4), (F1, G1, H2, I4), (F2, G1, H2, I4), (F3, G1, H2, I4),
(F4, G1, H2, I4), (F5, G1, H2, I4), (F1, G2, H2, I4), (F2, G2, H2, I4), (F3, G2, H2, I4), (F4, G2, H2, I4), (F5, G2,
H2, I4), (F1, G3, H2, I4), (F2, G3, H2, I4), (F3, G3, H2, I4), (F4, G3, H2, I4), (F5, G3, H2, I4), (F1, G4, H2, I4),
(F2, G4, H2, I4), (F3, G4, H2, I4), (F4, G4, H2, I4), (F5, G4, H2, I4), (F1, G1, H1, I5), (F2, G1, H1, I5), (F3, G1,
H1, I5), (F4, G1, H1, I5), (F5, G1, H1, I5), (F1, G2, H1, I5), (F2, G2, H1, I5), (F3, G2, H1, I5), (F4, G2, H1, I5),
(F5, G2, H1, I5), (F1, G3, H1, I5), (F2, G3, H1, I5), (F3, G3, H1, I5), (F4, G3, H1, I5), (F5, G3, H1, I5), (F1, G4,
H1, I5), (F2, G4, H1, I5), (F3, G4, H1, I5), (F4, G4, H1, I5), (F5, G4, H1, I5), (F1, G1, H2, I5), (F2, G1, H2, I5),
(F3, G1, H2, I5), (F4, G1, H2, I5), (F5, G1, H2, I5), (F1, G2, H2, I5), (F2, G2, H2, I5), (F3, G2, H2, I5), (F4, G2,
H2, I5), (F5, G2, H2, I5), (F1, G3, H2, I5), (F2, G3, H2, I5), (F3, G3, H2, I5), (F4, G3, H2, I5), (F5, G3, H2, I5),
(F1, G4, H2, I5), (F2, G4, H2, I5), (F3, G4, H2, I5), (F4, G4, H2, I5), (F5, G4, H2, I5), (F1, G1, H1, I1), (F2, G1,
H1, I1), (F3, G1, H1, I1), (F4, G1, H1, I1), (F5, G1, H1, I1), (F1, G2, H1, I1), (F2, G2, H1, I1), (F3, G2, H1, I1),
(F4, G2, H1, I1), (F5, G2, H1, I1), (F1, G3, H1, I1), (F2, G3, H1, I1), (F3, G3, H1, I1), (F4, G3, H1, I1), (F5, G3,
H1, I1), (F1, G4, H1, I1), (F2, G4, H1, I1), (F3, G4, H1, I1), (F4, G4, H1, I1), (F5, G4, H1, I1), (F1, G1, H2, I1),
(F2, G1, H2, I1), (F3, G1, H2, I1), (F4, G1, H2, I1), (F5, G1, H2, I1), (F1, G2, H2, I1), (F2, G2, H2, I1), (F3, G2,
H2, I1), (F4, G2, H2, I1), (F5, G2, H2, I1), (F1, G3, H2, I1), (F2, G3, H2, I1), (F3, G3, H2, I1), (F4, G3, H2, I1),
(F5, G3, H2, I1), (F1, G4, H2, I1), (F2, G4, H2, I1), (F3, G4, H2, I1), (F4, G4, H2, I1), (F5, G4, H2, I1), (F1, G1,
H1, I2), (F2, G1, H1, I2), (F3, G1, H1, I2), (F4, G1, H1, I2), (F5, G1, H1, I2), (F1, G2, H1, I2), (F2, G2, H1, I2),
(F3, G2, H1, I2), (F4, G2, H1, I2), (F5, G2, H1, I2), (F1, G3, H1, I2), (F2, G3, H1, I2), (F3, G3, H1, I2), (F4, G3,
H1, I2), (F5, G3, H1, I2), (F1, G4, H1, I2), (F2, G4, H1, I2), (F3, G4, H1, I2), (F4, G4, H1, I2), (F5, G4, H1, I2),
(F1, G1, H2, I2), (F2, G1, H2, I2), (F3, G1, H2, I2), (F4, G1, H2, I2), (F5, G1, H2, I2), (F1, G2, H2, I2), (F2, G2,
H2, I2), (F3, G2, H2, I2), (F4, G2, H2, I2), (F5, G2, H2, I2), (F1, G3, H2, I2), (F2, G3, H2, I2), (F3, G3, H2, I2),
(F4, G3, H2, I2), (F5, G3, H2, I2), (F1, G4, H2, I2), (F2, G4, H2, I2), (F3, G4, H2, I2), (F4, G4, H2, I2), (F5, G4,
H2, I2), (F1, G1, H1, I3), (F2, G1, H1, I3), (F3, G1, H1, I3), (F4, G1, H1, I3), (F5, G1, H1, I3), (F1, G2, H1, I3),
(F2, G2, H1, I3), (F3, G2, H1, I3), (F4, G2, H1, I3), (F5, G2, H1, I3), (F1, G3, H1, I3), (F2, G3, H1, I3), (F3, G3,
H1, I3), (F4, G3, H1, I3), (F5, G3, H1, I3), (F1, G4, H1, I3), (F2, G4, H1, I3), (F3, G4, H1, I3), (F4, G4, H1, I3),
(F5, G4, H1, I3), (F1, G1, H2, I3), (F2, G1, H2, I3), (F3, G1, H2, I3), (F4, G1, H2, I3), (F5, G1, H2, I3), (F1, G2,
H2, I3), (F2, G2, H2, I3), (F3, G2, H2, I3), (F4, G2, H2, I3), (F5, G2, H2, I3), (F1, G3, H2, I3), (F2, G3, H2, I3),
(F3, G3, H2, I3), (F4, G3, H2, I3), (F5, G3, H2, I3), (F1, G4, H2, I3), (F2, G4, H2, I3), (F3, G4, H2, I3), (F4, G4,
H2, I3), (F5, G4, H2, I3), (F1, G1, H1, I4), (F2, G1, H1, I4), (F3, G1, H1, I4), (F4, G1, H1, I4), (F5, G1, H1, I4),
(F1, G2, H1, I4), (F2, G2, H1, I4), (F3, G2, H1, I4), (F4, G2, H1, I4), (F5, G2, H1, I4), (F1, G3, H1, I4), (F2, G3,
H1, I4), (F3, G3, H1, I4), (F4, G3, H1, I4), (F5, G3, H1, I4), (F1, G4, H1, I4), (F2, G4, H1, I4), (F3, G4, H1, I4),
(F4, G4, H1, I4), (F5, G4, H1, I4), (F1, G1, H2, I4), (F2, G1, H2, I4), (F3, G1, H2, I4), (F4, G1, H2, I4), (F5, G1,
H2, I4), (F1, G2, H2, I4), (F2, G2, H2, I4), (F3, G2, H2, I4), (F4, G2, H2, I4), (F5, G2, H2, I4), (F1, G3, H2, I4),
(F2, G3, H2, I4), (F3, G3, H2, I4), (F4, G3, H2, I4), (F5, G3, H2, I4), (F1, G4, H2, I4), (F2, G4, H2, I4), (F3, G4,
H2, I4), (F4, G4, H2, I4), (F5, G4, H2, I4), (F1, G1, H1, I5), (F2, G1, H1, I5), (F3, G1, H1, I5), (F4, G1, H1, I5),
(F5, G1, H1, I5), (F1, G2, H1, I5), (F2, G2, H1, I5), (F3, G2, H1, I5), (F4, G2, H1, I5), (F5, G2, H1, I5), (F1, G3,
H1, I5), (F2, G3, H1, I5), (F3, G3, H1, I5), (F4, G3, H1, I5), (F5, G3, H1, I5), (F1, G4, H1, I5), (F2, G4, H1, I5),
(F3, G4, H1, I5), (F4, G4, H1, I5), (F5, G4, H1, I5), (F1, G1, H2, I5), (F2, G1, H2, I5), (F3, G1, H2, I5), (F4, G1,
H2, I5), (F5, G1, H2, I5), (F1, G2, H2, I5), (F2, G2, H2, I5), (F3, G2, H2, I5), (F4, G2, H2, I5), (F5, G2, H2, I5),
(F1, G3, H2, I5), (F2, G3, H2, I5), (F3, G3, H2, I5), (F4, G3, H2, I5), (F5, G3, H2, I5), (F1, G4, H2, I5), (F2, G4,
H2, I5), (F3, G4, H2, I5), (F4, G4, H2, I5), (F5, G4, H2, I5), (F1, G1, H1, I1), (F2, G1, H1, I1), (F3, G1, H1, I1),
(F4, G1, H1, I1), (F5, G1, H1, I1), (F1, G2, H1, I1), (F2, G2, H1, I1), (F3, G2, H1, I1), (F4, G2, H1, I1), (F5, G2,
H1, I1), (F1, G3, H1, I1), (F2, G3, H1, I1), (F3, G3, H1, I1), (F4, G3, H1, I1), (F5, G3, H1, I1), (F1, G4, H1, I1),
(F2, G4, H1, I1), (F3, G4, H1, I1), (F4, G4, H1, I1), (F5, G4, H1, I1), (F1, G1, H2, I1), (F2, G1, H2, I1), (F3, G1,
H2, I1), (F4, G1, H2, I1), (F5, G1, H2, I1), (F1, G2, H2, I1), (F2, G2, H2, I1), (F3, G2, H2, I1), (F4, G2, H2, I1),
(F5, G2, H2, I1), (F1, G3, H2, I1), (F2, G3, H2, I1), (F3, G3, H2, I1), (F4, G3, H2, I1), (F5, G3, H2, I1), (F1, G4,
H2, I1), (F2, G4, H2, I1), (F3, G4, H2, I1), (F4, G4, H2, I1), (F5, G4, H2, I1), (F1, G1, H1, I2), (F2, G1, H1, I2),
(F3, G1, H1, I2), (F4, G1, H1, I2), (F5, G1, H1, I2), (F1, G2, H1, I2), (F2, G2, H1, I2), (F3, G2, H1, I2), (F4, G2,
H1, I2), (F5, G2, H1, I2), (F1, G3, H1, I2), (F2, G3, H1, I2), (F3, G3, H1, I2), (F4, G3, H1, I2), (F5, G3, H1, I2),
(F1, G4, H1, I2), (F2, G4, H1, I2), (F3, G4, H1, I2), (F4, G4, H1, I2), (F5, G4, H1, I2), (F1, G1, H2, I2), (F2, G1,
H2, I2), (F3, G1, H2, I2), (F4, G1, H2, I2), (F5, G1, H2, I2), (F1, G2, H2, I2), (F2, G2, H2, I2), (F3, G2, H2, I2),
(F4, G2, H2, I2), (F5, G2, H2, I2), (F1, G3, H2, I2), (F2, G3, H2, I2), (F3, G3, H2, I2), (F4, G3, H2, I2), (F5, G3,
H2, I2), (F1, G4, H2, I2), (F2, G4, H2, I2), (F3, G4, H2, I2), (F4, G4, H2, I2), (F5, G4, H2, I2), (F1, G1, H1, I3),
(F2, G1, H1, I3), (F3, G1, H1, I3), (F4, G1, H1, I3), (F5, G1, H1, I3), (F1, G2, H1, I3), (F2, G2, H1, I3), (F3, G2,
H1, I3), (F4, G2, H1, I3), (F5, G2, H1, I3), (F1, G3, H1, I3), (F2, G3, H1, I3), (F3, G3, H1, I3), (F4, G3, H1, I3),
(F5, G3, H1, I3), (F1, G4, H1, I3), (F2, G4, H1, I3), (F3, G4, H1, I3), (F4, G4, H1, I3), (F5, G4, H1, I3), (F1, G1,
H2, I3), (F2, G1, H2, I3), (F3, G1, H2, I3), (F4, G1, H2, I3), (F5, G1, H2, I3), (F1, G2, H2, I3), (F2, G2, H2, I3),
(F3, G2, H2, I3), (F4, G2, H2, I3), (F5, G2, H2, I3), (F1, G3, H2, I3), (F2, G3, H2, I3), (F3, G3, H2, I3), (F4, G3,
H2, I3), (F5, G3, H2, I3), (F1, G4, H2, I3), (F2, G4, H2, I3), (F3, G4, H2, I3), (F4, G4, H2, I3), (F5, G4, H2, I3),
(F1, G1, H1, I4), (F2, G1, H1, I4), (F3, G1, H1, I4), (F4, G1, H1, I4), (F5, G1, H1, I4), (F1, G2, H1, I4), (F2, G2,
H1, I4), (F3, G2, H1, I4), (F4, G2, H1, I4), (F5, G2, H1, I4), (F1, G3, H1, I4), (F2, G3, H1, I4), (F3, G3, H1, I4),
(F4, G3, H1, I4), (F5, G3, H1, I4), (F1, G4, H1, I4), (F2, G4, H1, I4), (F3, G4, H1, I4), (F4, G4, H1, I4), (F5, G4,
H1, I4), (F1, G1, H2, I4), (F2, G1, H2, I4), (F3, G1, H2, I4), (F4, G1, H2, I4), (F5, G1, H2, I4), (F1, G2, H2, I4),
(F2, G2, H2, I4), (F3, G2, H2, I4), (F4, G2, H2, I4), (F5, G2, H2, I4), (F1, G3, H2, I4), (F2, G3, H2, I4), (F3, G3,
H2, I4), (F4, G3, H2, I4), (F5, G3, H2, I4), (F1, G4, H2, I4), (F2, G4, H2, I4), (F3, G4, H2, I4), (F4, G4, H2, I4),
(F5, G4, H2, I4), (F1, G1, H1, I5), (F2, G1, H1, I5), (F3, G1, H1, I5), (F4, G1, H1, I5), (F5, G1, H1, I5), (F1, G2,
H1, I5), (F2, G2, H1, I5), (F3, G2, H1, I5), (F4, G2, H1, I5), (F5, G2, H1, I5), (F1, G3, H1, I5), (F2, G3, H1, I5),
(F3, G3, H1, I5), (F4, G3, H1, I5), (F5, G3, H1, I5), (F1, G4, H1, I5), (F2, G4, H1, I5), (F3, G4, H1, I5), (F4, G4,
H1, I5), (F5, G4, H1, I5), (F1, G1, H2, I5), (F2, G1, H2, I5), (F3, G1, H2, I5), (F4, G1, H2, I5), (F5, G1, H2, I5),
(F1, G2, H2, I5), (F2, G2, H2, I5), (F3, G2, H2, I5), (F4, G2, H2, I5), (F5, G2, H2, I5), (F1, G3, H2, I5), (F2, G3,
H2, I5), (F3, G3, H2, I5), (F4, G3, H2, I5), (F5, G3, H2, I5), (F1, G4, H2, I5), (F2, G4, H2, I5), (F3, G4, H2, I5),
(F4, G4, H2, I5), (F5, G4, H2, I5), (F1, G1, H1, I1), (F2, G1, H1, I1), (F3, G1, H1, I1), (F4, G1, H1, I1), (F5, G1,
H1, I1), (F1, G2, H1, I1), (F2, G2, H1, I1), (F3, G2, H1, I1), (F4, G2, H1, I1), (F5, G2, H1, I1), (F1, G3, H1, I1),
(F2, G3, H1, I1), (F3, G3, H1, I1), (F4, G3, H1, I1), (F5, G3, H1, I1), (F1, G4, H1, I1), (F2, G4, H1, I1), (F3, G4,
H1, I1), (F4, G4, H1, I1), (F5, G4, H1, I1), (F1, G1, H2, I1), (F2, G1, H2, I1), (F3, G1, H2, I1), (F4, G1, H2, I1),
(F5, G1, H2, I1), (F1, G2, H2, I1), (F2, G2, H2, I1), (F3, G2, H2, I1), (F4, G2, H2, I1), (F5, G2, H2, I1), (F1, G3,
H2, I1), (F2, G3, H2, I1), (F3, G3, H2, I1), (F4, G3, H2, I1), (F5, G3, H2, I1), (F1, G4, H2, I1), (F2, G4, H2, I1),
(F3, G4, H2, I1), (F4, G4, H2, I1), (F5, G4, H2, I1), (F1, G1, H1, I2), (F2, G1, H1, I2), (F3, G1, H1, I2), (F4, G1,
H1, I2), (F5, G1, H1, I2), (F1, G2, H1, I2), (F2, G2, H1, I2), (F3, G2, H1, I2), (F4, G2, H1, I2), (F5, G2, H1, I2),
(F1, G3, H1, I2), (F2, G3, H1, I2), (F3, G3, H1, I2), (F4, G3, H1, I2), (F5, G3, H1, I2), (F1, G4, H1, I2), (F2, G4,
H1, I2), (F3, G4, H1, I2), (F4, G4, H1, I2), (F5, G4, H1, I2), (F1, G1, H2, I2), (F2, G1, H2, I2), (F3, G1, H2, I2),
(F4, G1, H2, I2), (F5, G1, H2, I2), (F1, G2, H2, I2), (F2, G2, H2, I2), (F3, G2, H2, I2), (F4, G2, H2, I2), (F5, G2,
H2, I2), (F1, G3, H2, I2), (F2, G3, H2, I2), (F3, G3, H2, I2), (F4, G3, H2, I2), (F5, G3, H2, I2), (F1, G4, H2, I2),
(F2, G4, H2, I2), (F3, G4, H2, I2), (F4, G4, H2, I2), (F5, G4, H2, I2), (F1, G1, H1, I3), (F2, G1, H1, I3), (F3, G1,
H1, I3), (F4, G1, H1, I3), (F5, G1, H1, I3), (F1, G2, H1, I3), (F2, G2, H1, I3), (F3, G2, H1, I3), (F4, G2, H1, I3),
(F5, G2, H1, I3), (F1, G3, H1, I3), (F2, G3, H1, I3), (F3, G3, H1, I3), (F4, G3, H1, I3), (F5, G3, H1, I3), (F1, G4,
H1, I3), (F2, G4, H1, I3), (F3, G4, H1, I3), (F4, G4, H1, I3), (F5, G4, H1, I3), (F1, G1, H2, I3), (F2, G1, H2, I3),
(F3, G1, H2, I3), (F4, G1, H2, I3), (F5, G1, H2, I3), F1, G2, H2, I3), (F2, G2, H2, I3), (F3, G2, H2, I3), (F4, G2,
H2, I3), (F5, G2, H2, I3), (F1, G3, H2, I3), (F2, G3, H2, I3), (F3, G3, H2, I3), (F4, G3, H2, I3), (F5, G3, H2, I3),
(F1, G4, H2, I3), (F2, G4, H2, I3), (F3, G4, H2, I3), (F4, G4, H2, I3), (F5, G4, H2, I3), (F1, G1, H1, I4), (F2, G1,
H1, I4), (F3, G1, H1, I4), (F4, G1, H1, I4), (F5, G1, H1, I4), (F1, G2, H1, I4), (F2, G2, H1, I4), (F3, G2, H1, I4),
(F4, G2, H1, I4), (F5, G2, H1, I4), (F1, G3, H1, I4), (F2, G3, H1, I4), (F3, G3, H1, I4), (F4, G3, H1, I4), (F5, G3,
H1, I4), (F1, G4, H1, I4), (F2, G4, H1, I4), (F3, G4, H1, I4), (F4, G4, H1, I4), (F5, G4, H1, I4), (F1, G1, H2, I4),
(F2, G1, H2, I4), (F3, G1, H2, I4), (F4, G1, H2, I4), (F5, G1, H2, I4), (F1, G2, H2, I4), (F2, G2, H2, I4), (F3, G2,
H2, I4), (F4, G2, H2, I4), (F5, G2, H2, I4), (F1, G3, H2, I4), (F2, G3, H2, I4), (F3, G3, H2, I4), (F4, G3, H2, I4),
(F5, G3, H2, I4), (F1, G4, H2, I4), (F2, G4, H2, I4), (F3, G4, H2, I4), (F4, G4, H2, I4), (F5, G4, H2, I4), (F1, G1,
H1, I5), (F2, G1, H1, I5), (F3, G1, H1, I5), (F4, G1, H1, I5), (F5, G1, H1, I5), (F1, G2, H1, I5), (F2, G2, H1, I5),
(F3, G2, H1, I5), (F4, G2, H1, I5), (F5, G2, H1, I5), (F1, G3, H1, I5), (F2, G3, H1, I5), (F3, G3, H1, I5), (F4, G3,
H1, I5), (F5, G3, H1, I5), (F1, G4, H1, I5), (F2, G4, H1, I5), (F3, G4, H1, I5), (F4, G4, H1, I5), (F5, G4, H1, I5),
(F1, G1, H2, I5), (F2, G1, H2, I5), (F3, G1, H2, I5), (F4, G1, H2, I5), (F5, G1, H2, I5), (F1, G2, H2, I5), (F2, G2,
H2, I5), (F3, G2, H2, I5), (F4, G2, H2, I5), (F5, G2, H2, I5), (F1, G3, H2, I5), (F2, G3, H2, I5), (F3, G3, H2, I5),
(F4, G3, H2, I5), (F5, G3, H2, I5), (F1, G4, H2, I5), (F2, G4, H2, I5), (F3, G4, H2, I5), (F4, G4, H2, I5), (F5, G4,
H2, I5), (F1, G1, H1, I1), (F2, G1, H1, I1), (F3, G1, H1, I1), (F4, G1, H1, I1), (F5, G1, H1, I1), (F1, G2, H1, I1),
(F2, G2, H1, I1), (F3, G2, H1, I1), (F4, G2, H1, I1), (F5, G2, H1, I1), (F1, G3, H1, I1), (F2, G3, H1, I1), (F3, G3,
H1, I1), (F4, G3, H1, I1), (F5, G3, H1, I1), (F1, G4, H1, I1), (F2, G4, H1, I1), (F3, G4, H1, I1), (F4, G4, H1, I1),
(F5, G4, H1, I1), (F1, G1, H2, I1), (F2, G1, H2, I1), (F3, G1, H2, I1), (F4, G1, H2, I1), (F5, G1, H2, I1), (F1, G2,
H2, I1), (F2, G2, H2, I1), (F3, G2, H2, I1), (F4, G2, H2, I1), (F5, G2, H2, I1), (F1, G3, H2, I1), (F2, G3, H2, I1),
(F3, G3, H2, I1), (F4, G3, H2, I1), (F5, G3, H2, I1), (F1, G4, H2, I1), (F2, G4, H2, I1), (F3, G4, H2, I1), (F4, G4,
H2, I1), (F5, G4, H2, I1), (F1, G1, H1, I2), (F2, G1, H1, I2), (F3, G1, H1, I2), (F4, G1, H1, I2), (F5, G1, H1, I2),
(F1, G2, H1, I2), (F2, G2, H1, I2), (F3, G2, H1, I2), (F4, G2, H1, I2), (F5, G2, H1, I2), (F1, G3, H1, I2), (F2, G3,
H1, I2), (F3, G3, H1, I2), (F4, G3, H1, I2), (F5, G3, H1, I2), (F1, G4, H1, I2), (F2, G4, H1, I2), (F3, G4, H1, I2),
(F4, G4, H1, I2), (F5, G4, H1, I2), (F1, G1, H2, I2), (F2, G1, H2, I2), (F3, G1, H2, I2), (F4, G1, H2, I2), (F5, G1,
H2, I2), (F1, G2, H2, I2), (F2, G2, H2, I2), (F3, G2, H2, I2), (F4, G2, H2, I2), (F5, G2, H2, I2), (F1, G3, H2, I2),
(F2, G3, H2, I2), (F3, G3, H2, I2), (F4, G3, H2, I2), (F5, G3, H2, I2), (F1, G4, H2, I2), (F2, G4, H2, I2), (F3, G4,
H2, I2), (F4, G4, H2, I2), (F5, G4, H2, I2), (F1, G1, H1, I3), (F2, G1, H1, I3), (F3, G1, H1, I3), (F4, G1, H1, I3),
(F5, G1, H1, I3), (F1, G2, H1, I3), (F2, G2, H1, I3), (F3, G2, H1, I3), (F4, G2, H1, I3), (F5, G2, H1, I3), (F1, G3,
H1, I3), (F2, G3, H1, I3), (F3, G3, H1, I3), (F4, G3, H1, I3), (F5, G3, H1, I3), (F1, G4, H1, I3), (F2, G4, H1, I3),
(F3, G4, H1, I3), (F4, G4, H1, I3), (F5, G4, H1, I3), (F1, G1, H2, I3), (F2, G1, H2, I3), (F3, G1, H2, I3), (F4, G1,
H2, I3), (F5, G1, H2, I3), (F1, G2, H2, I3), (F2, G2, H2, I3), (F3, G2, H2, I3), (F4, G2, H2, I3), (F5, G2, H2, I3),
(F1, G3, H2, I3), (F2, G3, H2, I3), (F3, G3, H2, I3), (F4, G3, H2, I3), (F5, G3, H2, I3), (F1, G4, H2, I3), (F2, G4,
H2, I3), (F3, G4, H2, I3), (F4, G4, H2, I3), (F5, G4, H2, I3), (F1, G1, H1, I4), (F2, G1, H1, I4), (F3, G1, H1, I4),
(F4, G1, H1, I4), (F5, G1, H1, I4), (F1, G2, H1, I4), (F2, G2, H1, I4), (F3, G2, H1, I4), (F4, G2, H1, I4), (F5, G2,
H1, I4), (F1, G3, H1, I4), (F2, G3, H1, I4), (F3, G3, H1, I4), (F4, G3, H1, I4), (F5, G3, H1, I4), (F1, G4, H1, I4),
(F2, G4, H1, I4), (F3, G4, H1, I4), (F4, G4, H1, I4), (F5, G4, H1, I4), (F1, G1, H2, I4), (F2, G1, H2, I4), (F3, G1,
H2, I4), (F4, G1, H2, I4), (F5, G1, H2, I4), (F1, G2, H2, I4), (F2, G2, H2, I4), (F3, G2, H2, I4), (F4, G2, H2, I4),
(F5, G2, H2, I4), (F1, G3, H2, I4), (F2, G3, H2, I4), (F3, G3, H2, I4), (F4, G3, H2, I4), (F5, G3, H2, I4), (F1, G4,
H2, I4), (F2, G4, H2, I4), (F3, G4, H2, I4), (F4, G4, H2, I4), (F5, G4, H2, I4), (F1, G1, H1, I5), (F2, G1, H1, I5),
(F3, G1, H1, I5), (F4, G1, H1, I5), (F5, G1, H1, I5), (F1, G2, H1, I5), (F2, G2, H1, I5), (F3, G2, H1, I5), (F4, G2,
H1, I5), (F5, G2, H1, I5), (F1, G3, H1, I5), (F2, G3, H1, I5), (F3, G3, H1, I5), (F4, G3, H1, I5), (F5, G3, H1, I5),
(F1, G4, H1, I5), (F2, G4, H1, I5), (F3, G4, H1, I5), (F4, G4, H1, I5), (F5, G4, H1, I5), (F1, G1, H2, I5), (F2, G1,
H2, I5), (F3, G1, H2, I5), (F4, G1, H2, I5), (F5, G1, H2, I5), (F1, G2, H2, I5), (F2, G2, H2, I5), (F3, G2, H2, I5),
(F4, G2, H2, I5), (F5, G2, H2, I5), (F1, G3, H2, I5), (F2, G3, H2, I5), (F3, G3, H2, I5), (F4, G3, H2, I5), (F5, G3,
H2, I5), (F1, G4, H2, I5), (F2, G4, H2, I5), (F3, G4, H2, I5), (F4, G4, H2, I5), and/or (F5, G4, H2, I5).

TABLE 7
Multiplexing of the Primer/Probe Combinations shown in Tables 2-3 for detection of S. aureus,
H. influenzae, M. catarrhalis, K. pneumoniae, and an internal amplification control (IAC)

(F1, G1, H1, I1, E1), (F2, G1, H1, I1, E1), (F3, G1, H1, I1, E1), (F4, G1, H1, I1, E1), (F5, G1, H1, I1, E1), (F1,
G2, H1, I1, E1), (F2, G2, H1, I1, E1), (F3, G2, H1, I1, E1), (F4, G2, H1, I1, E1), (F5, G2, H1, I1, E1), (F1, G3,
H1, I1, E1), (F2, G3, H1, I1, E1), (F3, G3, H1, I1, E1), (F4, G3, H1, I1, E1), (F5, G3, H1, I1, E1), (F1, G4, H1, I1,
E1), (F2, G4, H1, I1, E1), (F3, G4, H1, I1, E1), (F4, G4, H1, I1, E1), (F5, G4, H1, I1, E1), (F1, G1, H2, I1, E1),
(F2, G1, H2, I1, E1), (F3, G1, H2, I1, E1), (F4, G1, H2, I1, E1), (F5, G1, H2, I1, E1), (F1, G2, H2, I1, E1), (F2,
G2, H2, I1, E1), (F3, G2, H2, I1, E1), (F4, G2, H2, I1, E1), (F5, G2, H2, I1, E1), (F1, G3, H2, I1, E1), (F2, G3,
H2, I1, E1), (F3, G3, H2, I1, E1), (F4, G3, H2, I1, E1), (F5, G3, H2, I1, E1), (F1, G4, H2, I1, E1), (F2, G4, H2, I1,
E1), (F3, G4, H2, I1, E1), (F4, G4, H2, I1, E1), (F5, G4, H2, I1, E1), (F1, G1, H1, I2, E1), (F2, G1, H1, I2, E1),
(F3, G1, H1, I2, E1), (F4, G1, H1, I2, E1), (F5, G1, H1, I2, E1), (F1, G2, H1, I2, E1), (F2, G2, H1, I2, E1), (F3,
G2, H1, I2, E1), (F4, G2, H1, I2, E1), (F5, G2, H1, I2, E1), (F1, G3, H1, I2, E1), (F2, G3, H1, I2, E1), (F3, G3,
H1, I2, E1), (F4, G3, H1, I2, E1), (F5, G3, H1, I2, E1), (F1, G4, H1, I2, E1), (F2, G4, H1, I2, E1), (F3, G4, H1, I2,
E1), (F4, G4, H1, I2, E1), (F5, G4, H1, I2, E1), (F1, G1, H2, I2, E1), (F2, G1, H2, I2, E1), (F3, G1, H2, I2, E1),
(F4, G1, H2, I2, E1), (F5, G1, H2, I2, E1), (F1, G2, H2, I2, E1), (F2, G2, H2, I2, E1), (F3, G2, H2, I2, E1), (F4,
G2, H2, I2, E1), (F5, G2, H2, I2, E1), (F1, G3, H2, I2, E1), (F2, G3, H2, I2, E1), (F3, G3, H2, I2, E1), (F4, G3,
H2, I2, E1), (F5, G3, H2, I2, E1), (F1, G4, H2, I2, E1), (F2, G4, H2, I2, E1), (F3, G4, H2, I2, E1), (F4, G4, H2, I2,
E1), (F5, G4, H2, I2, E1), (F1, G1, H1, I3, E1), (F2, G1, H1, I3, E1), (F3, G1, H1, I3, E1), (F4, G1, H1, I3, E1),
(F5, G1, H1, I3, E1), (F1, G2, H1, I3, E1), (F2, G2, H1, I3, E1), (F3, G2, H1, I3, E1), (F4, G2, H1, I3, E1), (F5,
G2, H1, I3, E1), (F1, G3, H1, I3, E1), (F2, G3, H1, I3, E1), (F3, G3, H1, I3, E1), (F4, G3, H1, I3, E1), (F5, G3,
H1, I3, E1), (F1, G4, H1, I3, E1), (F2, G4, H1, I3, E1), (F3, G4, H1, I3, E1), (F4, G4, H1, I3, E1), (F5, G4, H1, I3,
E1), (F1, G1, H2, I3, E1), (F2, G1, H2, I3, E1), (F3, G1, H2, I3, E1), (F4, G1, H2, I3, E1), (F5, G1, H2, I3, E1),
(F1, G2, H2, I3, E1), (F2, G2, H2, I3, E1), (F3, G2, H2, I3, E1), (F4, G2, H2, I3, E1), (F5, G2, H2, I3, E1), (F1,
G3, H2, I3, E1), (F2, G3, H2, I3, E1), (F3, G3, H2, I3, E1), (F4, G3, H2, I3, E1), (F5, G3, H2, I3, E1), (F1, G4,
H2, I3, E1), (F2, G4, H2, I3, E1), (F3, G4, H2, I3, E1), (F4, G4, H2, I3, E1), (F5, G4, H2, I3, E1), (F1, G1, H1, I4,
E1), (F2, G1, H1, I4, E1), (F3, G1, H1, I4, E1), (F4, G1, H1, I4, E1), (F5, G1, H1, I4, E1), (F1, G2, H1, I4, E1),
(F2, G2, H1, I4, E1), (F3, G2, H1, I4, E1), (F4, G2, H1, I4, E1), (F5, G2, H1, I4, E1), (F1, G3, H1, I4, E1), (F2,
G3, H1, I4, E1), (F3, G3, H1, I4, E1), (F4, G3, H1, I4, E1), (F5, G3, H1, I4, E1), (F1, G4, H1, I4, E1), (F2, G4,
H1, I4, E1), (F3, G4, H1, I4, E1), (F4, G4, H1, I4, E1), (F5, G4, H1, I4, E1), (F1, G1, H2, I4, E1), (F2, G1, H2, I4,
E1), (F3, G1, H2, I4, E1), (F4, G1, H2, I4, E1), (F5, G1, H2, I4, E1), (F1, G2, H2, I4, E1), (F2, G2, H2, I4, E1),
(F3, G2, H2, I4, E1), (F4, G2, H2, I4, E1), (F5, G2, H2, I4, E1), (F1, G3, H2, I4, E1), (F2, G3, H2, I4, E1), (F3,
G3, H2, I4, E1), (F4, G3, H2, I4, E1), (F5, G3, H2, I4, E1), (F1, G4, H2, I4, E1), (F2, G4, H2, I4, E1), (F3, G4,
H2, I4, E1), (F4, G4, H2, I4, E1), (F5, G4, H2, I4, E1), (F1, G1, H1, I5, E1), (F2, G1, H1, I5, E1), (F3, G1, H1, I5,
E1), (F4, G1, H1, I5, E1), (F5, G1, H1, I5, E1), (F1, G2, H1, I5, E1), (F2, G2, H1, I5, E1), (F3, G2, H1, I5, E1),
(F4, G2, H1, I5, E1), (F5, G2, H1, I5, E1), (F1, G3, H1, I5, E1), (F2, G3, H1, I5, E1), (F3, G3, H1, I5, E1), (F4,
G3, H1, I5, E1), (F5, G3, H1, I5, E1), (F1, G4, H1, I5, E1), (F2, G4, H1, I5, E1), (F3, G4, H1, I5, E1), (F4, G4,
H1, I5, E1), (F5, G4, H1, I5, E1), (F1, G1, H2, I5, E1), (F2, G1, H2, I5, E1), (F3, G1, H2, I5, E1), (F4, G1, H2, I5,
E1), (F5, G1, H2, I5, E1), (F1, G2, H2, I5, E1), (F2, G2, H2, I5, E1), (F3, G2, H2, I5, E1), (F4, G2, H2, I5, E1),
(F5, G2, H2, I5, E1), (F1, G3, H2, I5, E1), (F2, G3, H2, I5, E1), (F3, G3, H2, I5, E1), (F4, G3, H2, I5, E1), (F5,
G3, H2, I5, E1), (F1, G4, H2, I5, E1), (F2, G4, H2, I5, E1), (F3, G4, H2, I5, E1), (F4, G4, H2, I5, E1), (F5, G4,
H2, I5, E1), (F1, G1, H1, I1, E2), (F2, G1, H1, I1, E2), (F3, G1, H1, I1, E2), (F4, G1, H1, I1, E2), (F5, G1, H1, I1,
E2), (F1, G2, H1, I1, E2), (F2, G2, H1, I1, E2), (F3, G2, H1, I1, E2), (F4, G2, H1, I1, E2), (F5, G2, H1, I1, E2),
(F1, G3, H1, I1, E2), (F2, G3, H1, I1, E2), (F3, G3, H1, I1, E2), (F4, G3, H1, I1, E2), (F5, G3, H1, I1, E2), (F1,
G4, H1, I1, E2), (F2, G4, H1, I1, E2), (F3, G4, H1, I1, E2), (F4, G4, H1, I1, E2), (F5, G4, H1, I1, E2), (F1, G1,
H2, I1, E2), (F2, G1, H2, I1, E2), (F3, G1, H2, I1, E2), (F4, G1, H2, I1, E2), (F5, G1, H2, I1, E2), (F1, G2, H2, I1,
E2), (F2, G2, H2, I1, E2), (F3, G2, H2, I1, E2), (F4, G2, H2, I1, E2), (F5, G2, H2, I1, E2), (F1, G3, H2, I1, E2),
(F2, G3, H2, I1, E2), (F3, G3, H2, I1, E2), (F4, G3, H2, I1, E2), (F5, G3, H2, I1, E2), (F1, G4, H2, I1, E2), (F2,
G4, H2, I1, E2), (F3, G4, H2, I1, E2), (F4, G4, H2, I1, E2), (F5, G4, H2, I1, E2), (F1, G1, H1, I2, E2), (F2, G1,
H1, I2, E2), (F3, G1, H1, I2, E2), (F4, G1, H1, I2, E2), (F5, G1, H1, I2, E2), (F1, G2, H1, I2, E2), (F2, G2, H1, I2,
E2), (F3, G2, H1, I2, E2), (F4, G2, H1, I2, E2), (F5, G2, H1, I2, E2), (F1, G3, H1, I2, E2), (F2, G3, H1, I2, E2),
(F3, G3, H1, I2, E2), (F4, G3, H1, I2, E2), (F5, G3, H1, I2, E2), (F1, G4, H1, I2, E2), (F2, G4, H1, I2, E2), (F3,
G4, H1, I2, E2), (F4, G4, H1, I2, E2), (F5, G4, H1, I2, E2), (F1, G1, H2, I2, E2), (F2, G1, H2, I2, E2), (F3, G1,
H2, I2, E2), (F4, G1, H2, I2, E2), (F5, G1, H2, I2, E2), (F1, G2, H2, I2, E2), (F2, G2, H2, I2, E2), (F3, G2, H2, I2,
E2), (F4, G2, H2, I2, E2), (F5, G2, H2, I2, E2), (F1, G3, H2, I2, E2), (F2, G3, H2, I2, E2), (F3, G3, H2, I2, E2),
(F4, G3, H2, I2, E2), (F5, G3, H2, I2, E2), (F1, G4, H2, I2, E2), (F2, G4, H2, I2, E2), (F3, G4, H2, I2, E2), (F4,
G4, H2, I2, E2), (F5, G4, H2, I2, E2), (F1, G1, H1, I3, E2), (F2, G1, H1, I3, E2), (F3, G1, H1, I3, E2), (F4, G1,
H1, I3, E2), (F5, G1, H1, I3, E2), (F1, G2, H1, I3, E2), (F2, G2, H1, I3, E2), (F3, G2, H1, I3, E2), (F4, G2, H1, I3,
E2), (F5, G2, H1, I3, E2), (F1, G3, H1, I3, E2), (F2, G3, H1, I3, E2), (F3, G3, H1, I3, E2), (F4, G3, H1, I3, E2),
(F5, G3, H1, I3, E2), (F1, G4, H1, I3, E2), (F2, G4, H1, I3, E2), (F3, G4, H1, I3, E2), (F4, G4, H1, I3, E2), (F5,
G4, H1, I3, E2), (F1, G1, H2, I3, E2), (F2, G1, H2, I3, E2), (F3, G1, H2, I3, E2), (F4, G1, H2, I3, E2), (F5, G1,
H2, I3, E2), (F1, G2, H2, I3, E2), (F2, G2, H2, I3, E2), (F3, G2, H2, I3, E2), (F4, G2, H2, I3, E2), (F5, G2, H2, I3,
E2), (F1, G3, H2, I3, E2), (F2, G3, H2, I3, E2), (F3, G3, H2, I3, E2), (F4, G3, H2, I3, E2), (F5, G3, H2, I3, E2),
(F1, G4, H2, I3, E2), (F2, G4, H2, I3, E2), (F3, G4, H2, I3, E2), (F4, G4, H2, I3, E2), (F5, G4, H2, I3, E2), (F1,
G1, H1, I4, E2), (F2, G1, H1, I4, E2), (F3, G1, H1, I4, E2), (F4, G1, H1, I4, E2), (F5, G1, H1, I4, E2), (F1, G2,
H1, I4, E2), (F2, G2, H1, I4, E2), (F3, G2, H1, I4, E2), (F4, G2, H1, I4, E2), (F5, G2, H1, I4, E2), (F1, G3, H1, I4,
E2), (F2, G3, H1, I4, E2), (F3, G3, H1, I4, E2), (F4, G3, H1, I4, E2), (F5, G3, H1, I4, E2), (F1, G4, H1, I4, E2),
(F2, G4, H1, I4, E2), (F3, G4, H1, I4, E2), (F4, G4, H1, I4, E2), (F5, G4, H1, I4, E2), (F1, G1, H2, I4, E2), (F2,
G1, H2, I4, E2), (F3, G1, H2, I4, E2), (F4, G1, H2, I4, E2), (F5, G1, H2, I4, E2), (F1, G2, H2, I4, E2), (F2, G2,
H2, I4, E2), (F3, G2, H2, I4, E2), (F4, G2, H2, I4, E2), (F5, G2, H2, I4, E2), (F1, G3, H2, I4, E2), (F2, G3, H2, I4,
E2), (F3, G3, H2, I4, E2), (F4, G3, H2, I4, E2), (F5, G3, H2, I4, E2), (F1, G4, H2, I4, E2), (F2, G4, H2, I4, E2),
(F3, G4, H2, I4, E2), (F4, G4, H2, I4, E2), (F5, G4, H2, I4, E2), (F1, G1, H1, I5, E2), (F2, G1, H1, I5, E2), (F3,
G1, H1, I5, E2), (F4, G1, H1, I5, E2), (F5, G1, H1, I5, E2), (F1, G2, H1, I5, E2), (F2, G2, H1, I5, E2), (F3, G2,
H1, I5, E2), (F4, G2, H1, I5, E2), (F5, G2, H1, I5, E 2), (F1, G3, H1, I5, E2), (F2, G3, H1, I5, E2), (F3, G3, H1,
15, E2), (F4, G3, H1, I5, E2), (F5, G3, H1, I5, E2), (F1, G4, H1, I5, E2), (F2, G4, H1, I5, E2), (F3, G4, H1, I5,
E2), (F4, G4, H1, I5, E2), (F5, G4, H1, I5, E2), (F1, G1, H2, I5, E2), (F2, G1, H2, I5, E2), (F3, G1, H2, I5, E2),
(F4, G1, H2, I5, E2), (F5, G1, H2, I5, E2), (F1, G2, H2, I5, E2), (F2, G2, H2, I5, E2), (F3, G2, H2, I5, E2), (F4,
G2, H2, I5, E2), (F5, G2, H2, I5, E2), (F1, G3, H2, I5, E2), (F2, G3, H2, I5, E2), (F3, G3, H2, I5, E2), (F4, G3,
H2, I5, E2), (F5, G3, H2, I5, E2), (F1, G4, H2, I5, E2), (F2, G4, H2, I5, E2), (F3, G4, H2, I5, E2), (F4, G4, H2, I5,
E2), (F5, G4, H2, I5, E2), (F1, G1, H1, I1, E3), (F2, G1, H1, I1, E3), (F3, G1, H1, I1, E3), (F4, G1, H1, I1, E3),
(F5, G1, H1, I1, E3), (F1, G2, H1, I1, E3), (F2, G2, H1, I1, E3), (F3, G2, H1, I1, E3), (F4, G2, H1, I1, E3), (F5,
G2, H1, I1, E3), (F1, G3, H1, I1, E3), (F2, G3, H1, I1, E3), (F3, G3, H1, I1, E3), (F4, G3, H1, I1, E3), (F5, G3,
H1, I1, E3), (F1, G4, H1, I1, E3), (F2, G4, H1, I1, E3), (F3, G4, H1, I1, E3), (F4, G4, H1, I1, E3), (F5, G4, H1, I1,
E3), (F1, G1, H2, I1, E3), (F2, G1, H2, I1, E3), (F3, G1, H2, I1, E3), (F4, G1, H2, I1, E3), (F5, G1, H2, I1, E3),
(F1, G2, H2, I1, E3), (F2, G2, H2, I1, E3), (F3, G2, H2, I1, E3), (F4, G2, H2, I1, E3), (F5, G2, H2, I1, E3), (F1,
G3, H2, I1, E3), (F2, G3, H2, I1, E3), (F3, G3, H2, I1, E3), (F4, G3, H2, I1, E3), (F5, G3, H2, I1, E3), (F1, G4,
H2, I1, E3), (F2, G4, H2, I1, E3), (F3, G4, H2, I1, E3), (F4, G4, H2, I1, E3), (F5, G4, H2, I1, E3), (F1, G1, H1, I2,
E3), (F2, G1, H1, I2, E3), (F3, G1, H1, I2, E3), (F4, G1, H1, I2, E3), (F5, G1, H1, I2, E3), (F1, G2, H1, I2, E3),
(F2, G2, H1, I2, E3), (F3, G2, H1, I2, E3), (F4, G2, H1, I2, E3), (F5, G2, H1, I2, E3), (F1, G3, H1, I2, E3), (F2,
G3, H1, I2, E3), (F3, G3, H1, I2, E3), (F4, G3, H1, I2, E3), (F5, G3, H1, I2, E3), (F1, G4, H1, I2, E3), (F2, G4,
H1, I2, E3), (F3, G4, H1, I2, E3), (F4, G4, H1, I2, E3), (F5, G4, H1, I2, E3), (F1, G1, H2, I2, E3), (F2, G1, H2, I2,
E3), (F3, G1, H2, I2, E3), (F4, G1, H2, I2, E3), (F5, G1, H2, I2, E3), (F1, G2, H2, I2, E3), (F2, G2, H2, I2, E3),
(F3, G2, H2, I2, E3), (F4, G2, H2, I2, E3), (F5, G2, H2, I2, E3), (F1, G3, H2, I2, E3), (F2, G3, H2, I2, E3), (F3,
G3, H2, I2, E3), (F4, G3, H2, I2, E3), (F5, G3, H2, I2, E3), (F1, G4, H2, I2, E3), (F2, G4, H2, I2, E3), (F3, G4,
H2, I2, E3), (F4, G4, H2, I2, E3), (F5, G4, H2, I2, E3), (F1, G1, H1, I3, E3), (F2, G1, H1, I3, E3), (F3, G1, H1, I3,
E3), (F4, G1, H1, I3, E3), (F5, G1, H1, I3, E3), (F1, G2, H1, I3, E3), (F2, G2, H1, I3, E3), (F3, G2, H1, I3, E3),
(F4, G2, H1, I3, E3), (F5, G2, H1, I3, E3), (F1, G3, H1, I3, E3), (F2, G3, H1, I3, E3), (F3, G3, H1, I3, E3), (F4,
G3, H1, I3, E3), (F5, G3, H1, I3, E3), (F1, G4, H1, I3, E3), (F2, G4, H1, I3, E3), (F3, G4, H1, I3, E3), (F4, G4,
H1, I3, E3), (F5, G4, H1, I3, E3), (F1, G1, H2, I3, E3), (F2, G1, H2, I3, E3), (F3, G1, H2, I3, E3), (F4, G1, H2, I3,
E3), (F5, G1, H2, I3, E3), (F1, G2, H2, I3, E3), (F2, G2, H2, I3, E3), (F3, G2, H2, I3, E3), (F4, G2, H2, I3, E3),
(F5, G2, H2, I3, E3), (F1, G3, H2, I3, E3), (F2, G3, H2, I3, E3), (F3, G3, H2, I3, E3), (F4, G3, H2, I3, E3), (F5,
G3, H2, I3, E3), (F1, G4, H2, I3, E3), (F2, G4, H2, I3, E3), (F3, G4, H2, I3, E3), (F4, G4, H2, I3, E3), (F5, G4,
H2, I3, E3), (F1, G1, H1, I4, E3), (F2, G1, H1, I4, E3), (F3, G1, H1, I4, E3), (F4, G1, H1, I4, E3), (F5, G1, H1, I4,
E3), (F1, G2, H1, I4, E3), (F2, G2, H1, I4, E3), (F3, G2, H1, I4, E3), (F4, G2, H1, I4, E3), (F5, G2, H1, I4, E3),
(F1, G3, H1, I4, E3), (F2, G3, H1, I4, E3), (F3, G3, H1, I4, E3), (F4, G3, H1, I4, E3), (F5, G3, H1, I4, E3), (F1,
G4, H1, I4, E3), (F2, G4, H1, I4, E3), (F3, G4, H1, I4, E3), (F4, G4, H1, I4, E3), (F5, G4, H1, I4, E3), (F1, G1,
H2, I4, E3), (F2, G1, H2, I4, E3), (F3, G1, H2, I4, E3), (F4, G1, H2, I4, E3), (F5, G1, H2, I4, E3), (F1, G2, H2, I4,
E3), (F2, G2, H2, I4, E3), (F3, G2, H2, I4, E3), (F4, G2, H2, I4, E3), (F5, G2, H2, I4, E3), (F1, G3, H2, I4, E3),
(F2, G3, H2, I4, E3), (F3, G3, H2, I4, E3), (F4, G3, H2, I4, E3), (F5, G3, H2, I4, E3), (F1, G4, H2, I4, E3), (F2,
G4, H2, I4, E3), (F3, G4, H2, I4, E3), (F4, G4, H2, I4, E3), (F5, G4, H2, I4, E3), (F1, G1, H1, I5, E3), (F2, G1,
H1, I5, E3), (F3, G1, H1, I5, E3), (F4, G1, H1, I5, E3), (F5, G1, H1, I5, E3), (F1, G2, H1, I5, E3), (F2, G2, H1, I5,
E3), (F3, G2, H1, I5, E3), (F4, G2, H1, I5, E3), (F5, G2, H1, I5, E3), (F1, G3, H1, I5, E3), (F2, G3, H1, I5, E3),
(F3, G3, H1, I5, E3), (F4, G3, H1, I5, E3), (F5, G3, H1, I5, E3), (F1, G4, H1, I5, E3), (F2, G4, H1, I5, E3), (F3,
G4, H1, I5, E3), (F4, G4, H1, I5, E3), (F5, G4, H1, I5, E3), (F1, G1, H2, I5, E3), (F2, G1, H2, I5, E3), (F3, G1,
H2, I5, E3), (F4, G1, H2, I5, E3), (F5, G1, H2, I5, E3), (F1, G2, H2, I5, E3), (F2, G2, H2, I5, E3), (F3, G2, H2, I5,
E3), (F4, G2, H2, I5, E3), (F5, G2, H2, I5, E3), (F1, G3, H2, I5, E3), (F2, G3, H2, I5, E3), (F3, G3, H2, I5, E3),
(F4, G3, H2, I5, E3), (F5, G3, H2, I5, E3), (F1, G4, H2, I5, E3), (F2, G4, H2, I5, E3), (F3, G4, H2, I5, E3), (F4,
G4, H2, I5, E3), (F5, G4, H2, I5, E3), (F1, G1, H1, I1, E4), (F2, G1, H1, I1, E4), (F3, G1, H1, I1, E4), (F4, G1,
H1, I1, E4), (F5, G1, H1, I1, E4), (F1, G2, H1, I1, E4), (F2, G2, H1, I1, E4), (F3, G2, H1, I1, E4), (F4, G2, H1, I1,
E4), (F5, G2, H1, I1, E4), (F1, G3, H1, I1, E4), (F2, G3, H1, I1, E4), (F3, G3, H1, I1, E4), (F4, G3, H1, I1, E4),
(F5, G3, H1, I1, E4), (F1, G4, H1, I1, E4), (F2, G4, H1, I1, E4), (F3, G4, H1, I1, E4), (F4, G4, H1, I1, E4), (F5,
G4, H1, I1, E4), (F1, G1, H2, I1, E4), (F2, G1, H2, I1, E4), (F3, G1, H2, I1, E4), (F4, G1, H2, I1, E4), (F5, G1,
H2, I1, E4), (F1, G2, H2, I1, E4), (F2, G2, H2, I1, E4), (F3, G2, H2, I1, E4), (F4, G2, H2, I1, E4), (F5, G2, H2, I1,
E4), (F1, G3, H2, I1, E4), (F2, G3, H2, I1, E4), (F3, G3, H2, I1, E4), (F4, G3, H2, I1, E4), (F5, G3, H2, I1, E4),
(F1, G4, H2, I1, E4), (F2, G4, H2, I1, E4), (F3, G4, H2, I1, E4), (F4, G4, H2, I1, E4), (F5, G4, H2, I1, E4), (F1,
G1, H1, I2, E4), (F2, G1, H1, I2, E4), (F3, G1, H1, I2, E4), (F4, G1, H1, I2, E4), (F5, G1, H1, I2, E4), (F1, G2,
H1, I2, E4), (F2, G2, H1, I2, E4), (F3, G2, H1, I2, E4), (F4, G2, H1, I2, E4), (F5, G2, H1, I2, E4), (F1, G3, H1, I2,
E4), (F2, G3, H1, I2, E4), (F3, G3, H1, I2, E4), (F4, G3, H1, I2, E4), (F5, G3, H1, I2, E4), (F1, G4, H1, I2, E4),
(F2, G4, H1, I2, E4), (F3, G4, H1, I2, E4), (F4, G4, H1, I2, E4), (F5, G4, H1, I2, E4), (F1, G1, H2, I2, E4), (F2,
G1, H2, I2, E4), (F3, G1, H2, I2, E4), (F4, G1, H2, I2, E4), (F5, G1, H2, I2, E4), (F1, G2, H2, I2, E4), (F2, G2,
H2, I2, E4), (F3, G2, H2, I2, E4), (F4, G2, H2, I2, E4), (F5, G2, H2, I2, E4), (F1, G3, H2, I2, E4), (F2, G3, H2, I2,
E4), (F3, G3, H2, I2, E4), (F4, G3, H2, I2, E4), (F5, G3, H2, I2, E4), (F1, G4, H2, I2, E4), (F2, G4, H2, I2, E4),
(F3, G4, H2, I2, E4), (F4, G4, H2, I2, E4), (F5, G4, H2, I2, E4), (F1, G1, H1, I3, E4), (F2, G1, H1, I3, E4), (F3,
G1, H1, I3, E4), (F4, G1, H1, I3, E4), (F5, G1, H1, I3, E4), (F1, G2, H1, I3, E4), (F2, G2, H1, I3, E4), (F3, G2,
H1, I3, E4), (F4, G2, H1, I3, E4), (F5, G2, H1, I3, E4), (F1, G3, H1, I3, E4), (F2, G3, H1, I3, E4), (F3, G3, H1, I3,
E4), (F4, G3, H1, I3, E4), (F5, G3, H1, I3, E4), (F1, G4, H1, I3, E4), (F2, G4, H1, I3, E4), (F3, G4, H1, I3, E4),
(F4, G4, H1, I3, E4), (F5, G4, H1, I3, E4), (F1, G1, H2, I3, E4), (F2, G1, H2, I3, E4), (F3, G1, H2, I3, E4), (F4,
G1, H2, I3, E4), (F5, G1, H2, I3, E4), F1, G2, H2, I3, E4), (F2, G2, H2, I3, E4), (F3, G2, H2, I3, E4), (F4, G2, H2,
13, E4), (F5, G2, H2, I3, E4), (F1, G3, H2, I3, E4), (F2, G3, H2, I3, E4), (F3, G3, H2, I3, E4), (F4, G3, H2, I3,
E4), (F5, G3, H2, I3, E4), (F1, G4, H2, I3, E4), (F2, G4, H2, I3, E4), (F3, G4, H2, I3, E4), (F4, G4, H2, I3, E4),
(F5, G4, H2, I3, E4), (F1, G1, H1, I4, E4), (F2, G1, H1, I4, E4), (F3, G1, H1, I4, E4), (F4, G1, H1, I4, E4), (F5,
G1, H1, I4, E4), (F1, G2, H1, I4, E4), (F2, G2, H1, I4, E4), (F3, G2, H1, I4, E4), (F4, G2, H1, I4, E4), (F5, G2,
H1, I4, E4), (F1, G3, H1, I4, E4), (F2, G3, H1, I4, E4), (F3, G3, H1, I4, E4), (F4, G3, H1, I4, E4), (F5, G3, H1, I4,
E4), (F1, G4, H1, I4, E4), (F2, G4, H1, I4, E4), (F3, G4, H1, I4, E4), (F4, G4, H1, I4, E4), (F5, G4, H1, I4, E4),
(F1, G1, H2, I4, E4), (F2, G1, H2, I4, E4), (F3, G1, H2, I4, E4), (F4, G1, H2, I4, E4), (F5, G1, H2, I4, E4), (F1,
G2, H2, I4, E4), (F2, G2, H2, I4, E4), (F3, G2, H2, I4, E4), (F4, G2, H2, I4, E4), (F5, G2, H2, I4, E4), (F1, G3,
H2, I4, E4), (F2, G3, H2, I4, E4), (F3, G3, H2, I4, E4), (F4, G3, H2, I4, E4), (F5, G3, H2, I4, E4), (F1, G4, H2, I4,
E4), (F2, G4, H2, I4, E4), (F3, G4, H2, I4, E4), (F4, G4, H2, I4, E4), (F5, G4, H2, I4, E4), (F1, G1, H1, I5, E4),
(F2, G1, H1, I5, E4), (F3, G1, H1, I5, E4), (F4, G1, H1, I5, E4), (F5, G1, H1, I5, E4), (F1, G2, H1, I5, E4), (F2,
G2, H1, I5, E4), (F3, G2, H1, I5, E4), (F4, G2, H1, I5, E4), (F5, G2, H1, I5, E4), (F1, G3, H1, I5, E4), (F2, G3,
H1, I5, E4), (F3, G3, H1, I5, E4), (F4, G3, H1, I5, E4), (F5, G3, H1, I5, E4), (F1, G4, H1, I5, E4), (F2, G4, H1, I5,
E4), (F3, G4, H1, I5, E4), (F4, G4, H1, I5, E4), (F5, G4, H1, I5, E4), (F1, G1, H2, I5, E4), (F2, G1, H2, I5, E4),
(F3, G1, H2, I5, E4), (F4, G1, H2, I5, E4), (F5, G1, H2, I5, E4), (F1, G2, H2, I5, E4), (F2, G2, H2, I5, E4), (F3,
G2, H2, I5, E4), (F4, G2, H2, I5, E4), (F5, G2, H2, I5, E4), (F1, G3, H2, I5, E4), (F2, G3, H2, I5, E4), (F3, G3,
H2, I5, E4), (F4, G3, H2, I5, E4), (F5, G3, H2, I5, E4), (F1, G4, H2, I5, E4), (F2, G4, H2, I5, E4), (F3, G4, H2, I5,
E4), (F4, G4, H2, I5, E4), (F5, G4, H2, I5, E4), (F1, G1, H1, I1, E5), (F2, G1, H1, I1, E5), (F3, G1, H1, I1, E5),
(F4, G1, H1, I1, E5), (F5, G1, H1, I1, E5), (F1, G2, H1, I1, E5), (F2, G2, H1, I1, E5), (F3, G2, H1, I1, E5), (F4,
G2, H1, I1, E5), (F5, G2, H1, I1, E5), (F1, G3, H1, I1, E5), (F2, G3, H1, I1, E5), (F3, G3, H1, I1, E5), (F4, G3,
H1, I1, E5), (F5, G3, H1, I1, E5), (F1, G4, H1, I1, E5), (F2, G4, H1, I1, E5), (F3, G4, H1, I1, E5), (F4, G4, H1, I1,
E5), (F5, G4, H1, I1, E5), (F1, G1, H2, I1, E5), (F2, G1, H2, I1, E5), (F3, G1, H2, I1, E5), (F4, G1, H2, I1, E5),
(F5, G1, H2, I1, E5), (F1, G2, H2, I1, E5), (F2, G2, H2, I1, E5), (F3, G2, H2, I1, E5), (F4, G2, H2, I1, E5), (F5,
G2, H2, I1, E5), (F1, G3, H2, I1, E5), (F2, G3, H2, I1, E5), (F3, G3, H2, I1, E5), (F4, G3, H2, I1, E5), (F5, G3,
H2, I1, E5), (F1, G4, H2, I1, E5), (F2, G4, H2, I1, E5), (F3, G4, H2, I1, E5), (F4, G4, H2, I1, E5), (F5, G4, H2, I1,
E5), (F1, G1, H1, I2, E5), (F2, G1, H1, I2, E5), (F3, G1, H1, I2, E5), (F4, G1, H1, I2, E5), (F5, G1, H1, I2, E5),
(F1, G2, H1, I2, E5), (F2, G2, H1, I2, E5), (F3, G2, H1, I2, E5), (F4, G2, H1, I2, E5), (F5, G2, H1, I2, E5), (F1,
G3, H1, I2, E5), (F2, G3, H1, I2, E5), (F3, G3, H1, I2, E5), (F4, G3, H1, I2, E5), (F5, G3, H1, I2, E5), (F1, G4,
H1, I2, E5), (F2, G4, H1, I2, E5), (F3, G4, H1, I2, E5), (F4, G4, H1, I2, E5), (F5, G4, H1, I2, E5), (F1, G1, H2, I2,
E5), (F2, G1, H2, I2, E5), (F3, G1, H2, I2, E5), (F4, G1, H2, I2, E5), (F5, G1, H2, I2, E5), (F1, G2, H2, I2, E5),
(F2, G2, H2, I2, E5), (F3, G2, H2, I2, E5), (F4, G2, H2, I2, E5), (F5, G2, H2, I2, E5), (F1, G3, H2, I2, E5), (F2,
G3, H2, I2, E5), (F3, G3, H2, I2, E5), (F4, G3, H2, I2, E5), (F5, G3, H2, I2, E5), (F1, G4, H2, I2, E5), (F2, G4,
H2, I2, E5), (F3, G4, H2, I2, E5), (F4, G4, H2, I2, E5), (F5, G4, H2, I2, E5), (F1, G1, H1, I3, E5), (F2, G1, H1, I3,
E5), (F3, G1, H1, I3, E5), (F4, G1, H1, I3, E5), (F5, G1, H1, I3, E5), (F1, G2, H1, I3, E5), (F2, G2, H1, I3, E5),
(F3, G2, H1, I3, E5), (F4, G2, H1, I3, E5), (F5, G2, H1, I3, E5), (F1, G3, H1, I3, E5), (F2, G3, H1, I3, E5), (F3,
G3, H1, I3, E5), (F4, G3, H1, I3, E5), (F5, G3, H1, I3, E5), (F1, G4, H1, I3, E5), (F2, G4, H1, I3, E5), (F3, G4,
H1, I3, E5), (F4, G4, H1, I3, E5), (F5, G4, H1, I3, E5), (F1, G1, H2, I3, E5), (F2, G1, H2, I3, E5), (F3, G1, H2, I3,
E5), (F4, G1, H2, I3, E5), (F5, G1, H2, I3, E5), (F1, G2, H2, I3, E5), (F2, G2, H2, I3, E5), (F3, G2, H2, I3, E5),
(F4, G2, H2, I3, E5), (F5, G2, H2, I3, E5), (F1, G3, H2, I3, E5), (F2, G3, H2, I3, E5), (F3, G3, H2, I3, E5), (F4,
G3, H2, I3, E5), (F5, G3, H2, I3, E5), (F1, G4, H2, I3, E5), (F2, G4, H2, I3, E5), (F3, G4, H2, I3, E5), (F4, G4,
H2, I3, E5), (F5, G4, H2, I3, E5), (F1, G1, H1, I4, E5), (F2, G1, H1, I4, E5), (F3, G1, H1, I4, E5), (F4, G1, H1, I4,
E5), (F5, G1, H1, I4, E5), (F1, G2, H1, I4, E5), (F2, G2, H1, I4, E5), (F3, G2, H1, I4, E5), (F4, G2, H1, I4, E5),
(F5, G2, H1, I4, E5), (F1, G3, H1, I4, E5), (F2, G3, H1, I4, E5), (F3, G3, H1, I4, E5), (F4, G3, H1, I4, E5), (F5,
G3, H1, I4, E5), (F1, G4, H1, I4, E5), (F2, G4, H1, I4, E5), (F3, G4, H1, I4, E5), (F4, G4, H1, I4, E5), (F5, G4,
H1, I4, E5), (F1, G1, H2, I4, E5), (F2, G1, H2, I4, E5), (F3, G1, H2, I4, E5), (F4, G1, H2, I4, E5), (F5, G1, H2, I4,
E5), (F1, G2, H2, I4, E5), (F2, G2, H2, I4, E5), (F3, G2, H2, I4, E5), (F4, G2, H2, I4, E5), (F5, G2, H2, I4, E5),
(F1, G3, H2, I4, E5), (F2, G3, H2, I4, E5), (F3, G3, H2, I4, E5), (F4, G3, H2, I4, E5), (F5, G3, H2, I4, E5), (F1,
G4, H2, I4, E5), (F2, G4, H2, I4, E5), (F3, G4, H2, I4, E5), (F4, G4, H2, I4, E5), (F5, G4, H2, I4, E5), (F1, G1,
H1, I5, E5), (F2, G1, H1, I5, E5), (F3, G1, H1, I5, E5), (F4, G1, H1, I5, E5), (F5, G1, H1, I5, E5), (F1, G2, H1, I5,
E5), (F2, G2, H1, I5, E5), (F3, G2, H1, I5, E5), (F4, G2, H1, I5, E5), (F5, G2, H1, I5, E5), (F1, G3, H1, I5, E5),
(F2, G3, H1, I5, E5), (F3, G3, H1, I5, E5), (F4, G3, H1, I5, E5), (F5, G3, H1, I5, E5), (F1, G4, H1, I5, E5), (F2,
G4, H1, I5, E5), (F3, G4, H1, I5, E5), (F4, G4, H1, I5, E5), (F5, G4, H1, I5, E5), (F1, G1, H2, I5, E5), (F2, G1,
H2, I5, E5), (F3, G1, H2, I5, E5), (F4, G1, H2, I5, E5), (F5, G1, H2, I5, E5), (F1, G2, H2, I5, E5), (F2, G2, H2, I5,
E5), (F3, G2, H2, I5, E5), (F4, G2, H2, I5, E5), (F5, G2, H2, I5, E5), (F1, G3, H2, I5, E5), (F2, G3, H2, I5, E5),
(F3, G3, H2, I5, E5), (F4, G3, H2, I5, E5), (F5, G3, H2, I5, E5), (F1, G4, H2, I5, E5), (F2, G4, H2, I5, E5), (F3,
G4, H2, I5, E5), (F4, G4, H2, I5, E5), and/or (F5, G4, H2, I5, E5).

The nucleic acids provided herein can be in various forms. For example, in some embodiments, the nucleic acids are dissolved (either alone or in combination with various other nucleic acids) in solution, for example buffer. In some embodiments, nucleic acids are provided, either alone or in combination with other isolated nucleic acids, as a salt. In some embodiments, nucleic acids are provided in a lyophilized form that can be reconstituted. For example, in some embodiments, the isolated nucleic acids disclosed herein can be provided in a lyophilized pellet alone, or in a lyophilized pellet with other isolated nucleic acids. In some embodiments, nucleic acids are provided affixed to a solid substance, such as a bead, a membrane, or the like. In some embodiments, nucleic acids are provided in a host cell, for example a cell line carrying a plasmid, or a cell line carrying a stably integrated sequence.

In some embodiments, the composition, reaction mixture, and kit comprise one or more pairs of amplification primers capable of specifically hybridizing to the sequence of the lytA gene, or a complement thereof, of S. pneumoniae. In some embodiments, the composition, reaction mixture, and kit comprise one or more probes capable of specifically hybridizing to the sequence of the lytA gene, or complement thereof, of S. pneumoniae. There are provided, in some embodiments, probes or primers up to about 100 nucleotides in length which is capable of hybridizing to the lytA gene of S. pneumoniae. In some embodiments, the probe or primer comprises: a sequence selected from the group consisting of SEQ ID NOs: 1-15, or sequence that exhibits at least about 85% identity, at least about 90% identity, or at least about 95% identity, to a sequence selected from the group consisting of SEQ ID NOs: 1-15. In some embodiments, said probe or primer consists of a sequence selected from the group consisting of SEQ ID NOs: 1-15, or sequence that exhibits at least about 85% identity, at least about 90% identity, or at least about 95% identity, to a sequence selected from the group consisting of SEQ ID NOs: 1-15. In some embodiments, said probe or primer comprises a sequence selected from the group consisting of SEQ ID NOs: 1-15. In some embodiments, said probe or primer consists of a sequence selected from the group consisting of SEQ ID NOs: 1-15.

In some embodiments, the composition, reaction mixture, and kit comprise one or more pairs of amplification primers capable of specifically hybridizing to the sequence of the cpsA gene, or a complement thereof, of S. pneumoniae. In some embodiments, the composition, reaction mixture, and kit comprise one or more probes capable of specifically hybridizing to the sequence of the cpsA gene, or complement thereof, of S. pneumoniae. There are provided, in some embodiments, probes or primers up to about 100 nucleotides in length which is capable of hybridizing to the cpsA gene of S. pneumoniae. In some embodiments, the probe or primer comprises: a sequence selected from the group consisting of SEQ ID NOs: 16-30, or sequence that exhibits at least about 85% identity, at least about 90% identity, or at least about 95% identity, to a sequence selected from the group consisting of SEQ ID NOs: 16-30. In some embodiments, said probe or primer consists of a sequence selected from the group consisting of SEQ ID NOs: 16-30, or sequence that exhibits at least about 85% identity, at least about 90% identity, or at least about 95% identity, to a sequence selected from the group consisting of SEQ ID NOs: 16-30. In some embodiments, said probe or primer comprises a sequence selected from the group consisting of SEQ ID NOs: 16-30. In some embodiments, said probe or primer consists of a sequence selected from the group consisting of SEQ ID NOs: 16-30.

In some embodiments, the composition, reaction mixture, and kit comprise one or more pairs of amplification primers capable of specifically hybridizing to the sequence of the sodC gene, or a complement thereof, of N. meningitidis. In some embodiments, the composition, reaction mixture, and kit comprise one or more probes capable of specifically hybridizing to the sequence of the sodC gene, or complement thereof, of N. meningitidis. There are provided, in some embodiments, probes or primers up to about 100 nucleotides in length which is capable of hybridizing to the sodC gene of N. meningitidis. In some embodiments, the probe or primer comprises: a sequence selected from the group consisting of SEQ ID NOs: 31-44, or sequence that exhibits at least about 85% identity, at least about 90% identity, or at least about 95% identity, to a sequence selected from the group consisting of SEQ ID NOs: 31-44. In some embodiments, said probe or primer consists of a sequence selected from the group consisting of SEQ ID NOs: 31-44, or sequence that exhibits at least about 85% identity, at least about 90% identity, or at least about 95% identity, to a sequence selected from the group consisting of SEQ ID NOs: 31-44. In some embodiments, said probe or primer comprises a sequence selected from the group consisting of SEQ ID NOs: 31-44. In some embodiments, said probe or primer consists of a sequence selected from the group consisting of SEQ ID NOs: 31-44.

In some embodiments, the composition, reaction mixture, and kit comprise one or more pairs of amplification primers capable of specifically hybridizing to the sequence of the crtA gene, or a complement thereof, of N. meningitidis. In some embodiments, the composition, reaction mixture, and kit comprise one or more probes capable of specifically hybridizing to the sequence of the crtA gene, or complement thereof, of N. meningitidis. There are provided, in some embodiments, probes or primers up to about 100 nucleotides in length which is capable of hybridizing to the crtA gene of N. meningitidis. In some embodiments, the probe or primer comprises: a sequence selected from the group consisting of SEQ ID NOs: 45-59, or sequence that exhibits at least about 85% identity, at least about 90% identity, or at least about 95% identity, to a sequence selected from the group consisting of SEQ ID NOs: 45-59. In some embodiments, said probe or primer consists of a sequence selected from the group consisting of SEQ ID NOs: 45-59, or sequence that exhibits at least about 85% identity, at least about 90% identity, or at least about 95% identity, to a sequence selected from the group consisting of SEQ ID NOs: 45-59. In some embodiments, said probe or primer comprises a sequence selected from the group consisting of SEQ ID NOs: 45-59. In some embodiments, said probe or primer consists of a sequence selected from the group consisting of SEQ ID NOs: 45-59.

In some embodiments, the composition, reaction mixture, and kit comprise one or more pairs of amplification primers capable of specifically hybridizing to the sequence of an internal amplification control (IAC), or a complement thereof. In some embodiments, the composition, reaction mixture, and kit comprise one or more probes capable of specifically hybridizing to the sequence of an internal amplification control (IAC), or complement thereof. There are provided, in some embodiments, probes or primers up to about 100 nucleotides in length which is capable of hybridizing to an internal amplification control (IAC). In some embodiments, the probe or primer comprises: a sequence selected from the group consisting of SEQ ID NOs: 60-74 and 130-134, or sequence that exhibits at least about 85% identity, at least about 90% identity, or at least about 95% identity, to a sequence selected from the group consisting of SEQ ID NOs: 60-74 and 130-134. In some embodiments, said probe or primer consists of a sequence selected from the group consisting of SEQ ID NOs: 60-74 and 130-134, or sequence that exhibits at least about 85% identity, at least about 90% identity, or at least about 95% identity, to a sequence selected from the group consisting of SEQ ID NOs: 60-74 and 130-134. In some embodiments, said probe or primer comprises a sequence selected from the group consisting of SEQ ID NOs: 60-74 and 130-134. In some embodiments, said probe or primer consists of a sequence selected from the group consisting of SEQ ID NOs: 60-74 and 130-134.

In some embodiments, the composition, reaction mixture, and kit comprise one or more pairs of amplification primers capable of specifically hybridizing to the sequence of the nuc gene, or a complement thereof, of S. aureus. In some embodiments, the composition, reaction mixture, and kit comprise one or more probes capable of specifically hybridizing to the sequence of the nuc gene, or complement thereof, of S. aureus. There are provided, in some embodiments, probes or primers up to about 100 nucleotides in length which is capable of hybridizing to the nuc gene of S. aureus. In some embodiments, the probe or primer comprises: a sequence selected from the group consisting of SEQ ID NOs: 75-87, or sequence that exhibits at least about 85% identity, at least about 90% identity, or at least about 95% identity, to a sequence selected from the group consisting of SEQ ID NOs: 75-87. In some embodiments, said probe or primer consists of a sequence selected from the group consisting of SEQ ID NOs: 75-87, or sequence that exhibits at least about 85% identity, at least about 90% identity, or at least about 95% identity, to a sequence selected from the group consisting of SEQ ID NOs: 75-87. In some embodiments, said probe or primer comprises a sequence selected from the group consisting of SEQ ID NOs: 75-87. In some embodiments, said probe or primer consists of a sequence selected from the group consisting of SEQ ID NOs: 75-87.

In some embodiments, the composition, reaction mixture, and kit comprise one or more pairs of amplification primers capable of specifically hybridizing to the sequence of the fucK gene, or a complement thereof, of H. influenzae. In some embodiments, the composition, reaction mixture, and kit comprise one or more probes capable of specifically hybridizing to the sequence of the fucK gene, or complement thereof, of H. influenzae. There are provided, in some embodiments, probes or primers up to about 100 nucleotides in length which is capable of hybridizing to the fucK gene of H. influenzae. In some embodiments, the probe or primer comprises: a sequence selected from the group consisting of SEQ ID NOs: 88-100, or sequence that exhibits at least about 85% identity, at least about 90% identity, or at least about 95% identity, to a sequence selected from the group consisting of SEQ ID NOs: 88-100. In some embodiments, said probe or primer consists of a sequence selected from the group consisting of SEQ ID NOs: 88-100, or sequence that exhibits at least about 85% identity, at least about 90% identity, or at least about 95% identity, to a sequence selected from the group consisting of SEQ ID NOs: 88-100. In some embodiments, said probe or primer comprises a sequence selected from the group consisting of SEQ ID NOs: 88-100. In some embodiments, said probe or primer consists of a sequence selected from the group consisting of SEQ ID NOs: 88-100.

In some embodiments, the composition, reaction mixture, and kit comprise one or more pairs of amplification primers capable of specifically hybridizing to the sequence of the copB gene, or a complement thereof, of M. catarrhalis. In some embodiments, the composition, reaction mixture, and kit comprise one or more probes capable of specifically hybridizing to the sequence of the copB gene, or complement thereof, of M. catarrhalis. There are provided, in some embodiments, probes or primers up to about 100 nucleotides in length which is capable of hybridizing to the copB gene of M. catarrhalis. In some embodiments, the probe or primer comprises: a sequence selected from the group consisting of SEQ ID NOs: 101-115, or sequence that exhibits at least about 85% identity, at least about 90% identity, or at least about 95% identity, to a sequence selected from the group consisting of SEQ ID NOs: 101-115. In some embodiments, said probe or primer consists of a sequence selected from the group consisting of SEQ ID NOs: 101-115, or sequence that exhibits at least about 85% identity, at least about 90% identity, or at least about 95% identity, to a sequence selected from the group consisting of SEQ ID NOs: 101-115. In some embodiments, said probe or primer comprises a sequence selected from the group consisting of SEQ ID NOs: 101-115. In some embodiments, said probe or primer consists of a sequence selected from the group consisting of SEQ ID NOs: 101-115.

In some embodiments, the composition, reaction mixture, and kit comprise one or more pairs of amplification primers capable of specifically hybridizing to the sequence of the gltA gene, or a complement thereof, of K. pneumoniae. In some embodiments, the composition, reaction mixture, and kit comprise one or more probes capable of specifically hybridizing to the sequence of the gltA gene, or complement thereof, of K. pneumoniae. There are provided, in some embodiments, probes or primers up to about 100 nucleotides in length which is capable of hybridizing to the gltA gene of K. pneumoniae. In some embodiments, the probe or primer comprises: a sequence selected from the group consisting of SEQ ID NOs: 116-129, or sequence that exhibits at least about 85% identity, at least about 90% identity, or at least about 95% identity, to a sequence selected from the group consisting of SEQ ID NOs: 116-129. In some embodiments, said probe or primer consists of a sequence selected from the group consisting of SEQ ID NOs: 116-129, or sequence that exhibits at least about 85% identity, at least about 90% identity, or at least about 95% identity, to a sequence selected from the group consisting of SEQ ID NOs: 116-129. In some embodiments, said probe or primer comprises a sequence selected from the group consisting of SEQ ID NOs: 116-129. In some embodiments, said probe or primer consists of a sequence selected from the group consisting of SEQ ID NOs: 116-129.

There are provided, in some embodiments, compositions comprising two or more of the oligonucleotide probes and/or primers disclosed herein.

Oligonucleotide probes can, in some embodiments, include a detectable moiety. For example, the oligonucleotide probes disclosed herein can comprise a radioactive label. Non-limiting examples of radioactive labels include ³H, ¹⁴C, ³²P, and ³⁵S. In some embodiments, oligonucleotide probes can include one or more non-radioactive detectable markers or moieties, including but not limited to ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe. For example, oligonucleotide probes labeled with one or more dyes, such that upon hybridization to a template nucleic acid, a detectable change in fluorescence is generated. While non-specific dyes may be desirable for some applications, sequence-specific probes can provide more accurate measurements of amplification. One configuration of sequence-specific probe can include one end of the probe tethered to a fluorophore, and the other end of the probe tethered to a quencher. When the probe is unhybridized, it can maintain a stem-loop configuration, in which the fluorophore is quenched by the quencher, thus preventing the fluorophore from fluorescing. When the probe is hybridized to a template nucleic sequence, it is linearized, distancing the fluorophore from the quencher, and thus permitting the fluorophore to fluoresce. Another configuration of sequence-specific probe can include a first probe tethered to a first fluorophore of a FRET pair, and a second probe tethered to a second fluorophore of a FRET pair. The first probe and second probe can be configured to hybridize to sequences of an amplicon that are within sufficient proximity to permit energy transfer by FRET when the first probe and second probe are hybridized to the same amplicon.

In some embodiments the probe is a TaqMan probe. TaqMan probes can comprise a fluorophore and a quencher. The quencher molecule can quench the fluorescence emitted by the fluorophore when excited by the cycler's light source via Förster resonance energy transfer (FRET). As long as the fluorophore and the quencher are in proximity, quenching can inhibit any detectable (e.g., fluorescence) signals. TaqMan probes provided herein can designed such that they anneal within a DNA region amplified by primers provided herein. Without being bound by any particular theory, in some embodiments, as a PCR polymerase (e.g., Taq) extends the primer and synthesizes a nascent strand on a single-strand template, the 5′ to 3′ exonuclease activity of the PCR polymerase degrades the probe that has annealed to the template. Degradation of the probe can release the fluorophore from it and break the proximity to the quencher, thereby relieving the quenching effect and allowing fluorescence of the fluorophore. Hence, fluorescence detected in the quantitative PCR thermal cycler can, in some embodiments, be directly proportional to the fluorophore released and the amount of DNA template present in the PCR.

In some embodiments, the sequence specific probe comprises an oligonucleotide as disclosed herein conjugated to a fluorophore. In some embodiments, the probe is conjugated to two or more fluorophores. Examples of fluorophores include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc.; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, CAL fluor orange, and the like. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Probes can comprise SpC6, or functional equivalents and derivatives thereof. Probes can comprise a spacer moiety. A spacer moiety can comprise an alkyl group of at least 2 carbons to about 12 carbons. A probe can comprise a spacer comprising an abasic unit. A probe can comprise a spacer selected from the group comprising of idSp, iSp9, iS18, iSpC3, iSpC6, iSpC12, or any combination thereof.

In some embodiments, the probe is conjugated to a quencher. A quencher can absorb electromagnetic radiation and dissipate it as heat, thus remaining dark. Example quenchers include Dabcyl, NFQ's, such as BHQ-1 or BHQ-2 (Biosearch), IOWA BLACK FQ (IDT), and IOWA BLACK RQ (IDT). In some embodiments, the quencher is selected to pair with a fluorophore so as to absorb electromagnetic radiation emitted by the fluorophore. Fluorophore/quencher pairs useful in the compositions and methods disclosed herein are well-known in the art, and can be found, e.g., described in Marras, “Selection of Fluorophore and Quencher Pairs for Fluorescent Nucleic Acid Hybridization Probes” available at www.molecular-beacons.org/download/marras,mmb06%28335%293.pdf. Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like. Examples of an ATTO quencher include, but are not limited to: ATTO 540Q, ATTO 580Q, and ATTO 612Q. Examples of a Black Hole Quencher® (BHQ®) include, but are not limited to: BHQ-0 (493 nm), BHQ-1 (534 nm), BHQ-2 (579 nm) and BHQ-3 (672 nm).

In some embodiments, a detectable label is a fluorescent label selected from: an Alexa Fluor® dye (e.g., Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, and Alexa Fluor® 790), an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rhol 1, ATTO Rhol2, ATTO Thiol 2, ATTO 590, ATTO 594, ATTO Rhol3, ATTO 610, ATTO 620, ATTO Rhol4, ATTO 633, ATTO 647, ATTO 647N, ATTO 655, ATTO Oxal2, ATTO 665, ATTO 680, ATTO 700, ATTO 725, and ATTO 740), a DyFight dye, a cyanine dye (e.g., Cy2, Cy3, Cy3.5, Cy3b, Cy5, Cy5.5, Cy7, and Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, Pacific Orange, a quantum dot, and a tethered fluorescent protein.

In some embodiments, a fluorophore is attached to a first end of the probe, and a quencher is attached to a second end of the probe. In some embodiments, a probe can comprise two or more fluorophores. In some embodiments, a probe can comprise two or more quencher moieties. In some embodiments, a probe can comprise one or more quencher moieties and/or one or more fluorophores. A quencher moiety or a fluorophore can be attached to any portion of a probe (e.g., on the 5′ end, on the 3′ end, and/or in the middle of the probe). Any probe nucleotide can comprise a fluorophore and/or a quencher moiety, such as, for example, iBHQ1dT. For example, with regards to F1-nuc-probe (TTTCGTAAATGCACTTGCTTCAGGACCA; SEQ ID NO: 84), any of the first, second, third, fourth, fifth, sixth, seventh, eighth, or ninth “T” can comprise a fluorophore or a quencher moiety (e.g., iBHQ1dT). Attachment can include covalent bonding, and can optionally include at least one linker molecule positioned between the probe and the fluorophore or quencher. In some embodiments, a fluorophore is attached to a 5′ end of a probe, and a quencher is attached to a 3′ end of a probe. In some embodiments, a fluorophore is attached to a 3′ end of a probe, and a quencher is attached to a 5′ end of a probe. Examples of probes that can be used in quantitative nucleic acid amplification include molecular beacons, SCORPION™ probes (Sigma), TAQMAN™ probes (Life Technologies) and the like. Other nucleic acid detection technologies that are useful in the embodiments disclosed herein include, but are not limited to nanoparticle probe technology (See, Elghanian, et al. (1997) Science 277:1078-1081.) and Amplifluor probe technology (See, U.S. Pat. Nos. 5,866,366; 6,090,592; 6,117,635; and 6,117,986).

There are provided, in some embodiments, compositions for detecting S. pneumoniae and N. meningitidis in a sample. In some embodiments, the composition comprises: at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity (e.g., 85%, 86%, 87%, 88%, 89%, 90%, 91%, 92%, 93%, 94%, 95%, 96%, 97% 98%, 99%, 100%, or a number or a range between any two of these values) to any one of the sequences of SEQ ID NOs: 1-10; at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 16-25; at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 31-39; and at least one pair of primers capable of hybridizing to the crtA gene of N. meningitidis, wherein each primer in said at least one pair of primers comprises any one of the sequences of SEQ ID NOs: 45-54, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 45-54. The composition can comprise: at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 60-69 and 130-132.

In some embodiments, the at least one pair of primers capable of hybridizing to the lytA gene of S. pneumoniae comprises a primer comprising the sequence of SEQ ID NOs: 1, 3, 5, 7, or 9 and a primer comprising the sequence of SEQ ID NOs: 2, 4, 6, 8, or 10; the at least one pair of primers capable of hybridizing to the cpsA gene of S. pneumoniae comprises a primer comprising the sequence of SEQ ID NOs: 16, 18, 20, 22, or 24 and a primer comprising the sequence of SEQ ID NOs: 17, 19, 21, 23, or 25; the at least one pair of primers capable of hybridizing to the sodC gene of N. meningitidis comprises a primer comprising the sequence of SEQ ID NOs: 31, 33, 35, 36, or 38 and a primer comprising the sequence of SEQ ID NOs: 32, 34, 37, or 39; and the at least one pair of primers capable of hybridizing to the crtA gene of N. meningitidis comprises a primer comprising the sequence of SEQ ID NOs: 45, 47, 49, 51, or 53 and a primer comprising the sequence of SEQ ID NOs: 46, 48, 50, 52, or 54. In some embodiments, the at least one pair of control primers capable of hybridizing to the IAC comprises a primer comprising the sequence of SEQ ID NOs: 60, 62, 64, 66, 68, or 131 and a primer comprising the sequence of SEQ ID NOs: 61, 63, 65, 67, 69, 130, or 132.

The composition can comprise: a plurality of oligonucleotide probes, wherein each of the plurality of oligonucleotide probes comprises a sequence selected from the group consisting of SEQ ID NOs: 11-15, 26-30, 40-44, 55-59, 70-74, and 133-134, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 11-15, 26-30, 40-44, 55-59, 70-74, and 133-134. Each of the plurality of oligonucleotide probes can comprise a sequence selected from the group consisting of SEQ ID NOs: 11-15, 26-30, 40-44, 55-59, 70-74, and 133-134. Each of the plurality of oligonucleotide probes can consist of a sequence selected from the group consisting of SEQ ID NOs: 11-15, 26-30, 40-44, 55-59, 70-74, and 133-134. At least one of the plurality of probes can comprise a fluorescence emitter moiety and a fluorescence quencher moiety.

There are provided, in some embodiments, compositions for detecting S. aureus, H. influenzae, M. catarrhalis, and K. pneumoniae in a sample. In some embodiments, the composition comprises: at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 75-83; at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 88-95; at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 101-110; and at least one pair of primers capable of hybridizing to the gltA gene of K. pneumoniae, wherein each primer in said at least one pair of primers comprises any one of the sequences of SEQ ID NOs: 116-124, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 116-124. The composition can comprise: at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity (e.g., 85%, 86%, 87%, 88%, 89%, 90%, 91%, 92%, 93%, 94%, 95%, 96%, 97%, 98%, 99% 100%, or a number or a range between any two of these values) to any one of the sequences of SEQ ID NOs: 60-69 and 130-132.

In some embodiments, the at least one pair of primers capable of hybridizing to the nuc gene of S. aureus comprises a primer comprising the sequence of SEQ ID NOs: 75, 77, 79, 81, or 83 and a primer comprising the sequence of SEQ ID NOs: 76, 78, 80, or 82; the at least one pair of primers capable of hybridizing to the fucK gene of H. influenzae comprises a primer comprising the sequence of SEQ ID NOs: 88, 90, 92, 93, or 94 and a primer comprising the sequence of SEQ ID NOs: 89, 91, or 95; the at least one pair of primers capable of hybridizing to the copB gene of M. catarrhalis comprises a primer comprising the sequence of SEQ ID NOs: 101, 103, 105, 107, or 109 and a primer comprising the sequence of SEQ ID NOs: 102, 104, 106, 108, or 110; and the at least one pair of primers capable of hybridizing to the gltA gene of K. pneumoniae comprises a primer comprising the sequence of SEQ ID NOs: 116, 118, 120, or 122 and a primer comprising the sequence of SEQ ID NOs: 117, 119, 121, 123, or 124. In some embodiments, the at least one pair of control primers capable of hybridizing to the IAC comprises a primer comprising the sequence of SEQ ID NOs: 60, 62, 64, 66, 68, or 131 and a primer comprising the sequence of SEQ ID NOs: 61, 63, 65, 67, 69, 130, or 132.

There are provided, in some embodiments, a plurality of oligonucleotide probes, wherein each of the plurality of oligonucleotide probes comprises a sequence selected from the group consisting of SEQ ID NOs: 84-87, 96-100, 111-115, 125-129, 70-74, and 133-134, or a sequence that exhibits at least about 85% identity (e.g., 85%, 86%, 87%, 88%, 89%, 90%, 91% 92%, 93%, 94%, 95%, 96% 97%, 98%, 99%, 100%, or a number or a range between any two of these values) to a sequence selected from the group consisting of SEQ ID NOs: 84-87, 96-100, 111-115, 125-129, 70-74, and 133-134. In some embodiments, each of the plurality of oligonucleotide probes comprises a sequence selected from the group consisting of SEQ ID NOs: 84-87, 96-100, 111-115, 125-129, 70-74, and 133-134. In some embodiments, each of the plurality of oligonucleotide probes consists of a sequence selected from the group consisting of SEQ ID NOs: 84-87, 96-100, 111-115, 125-129, 70-74, and 133-134. In some embodiments, at least one of the plurality of probes comprises a fluorescence emitter moiety and a fluorescence quencher moiety.

Any probes described herein can comprise a fluorescence emitter moiety, a fluorescence quencher moiety, or both.

As disclosed herein, a reaction mixture can comprise one or more of the primers disclosed herein, one or more of the probes disclosed herein (e.g., the fluorophore-containing probes), or any combination thereof. In some embodiments, the reaction mixture comprises one or more of the primer and/or probe-containing composition disclosed herein. The reaction mixture can also comprise various additional components. Examples of the additional components in the reaction mixture include, but are not limited to, template DNA, DNA polymerase (e.g., Taq DNA polymerase), deoxynucleotides (dNTPs), buffer solution, biovalent cations, monovalent cation potassium ions, and any combination thereof. In some embodiments, the reaction mixture is a master mix for real-time PCR.

### Samples

The methods and compositions disclosed herein are suitable for detecting bacterial respiratory pathogens, such as Streptococcus pneumoniae, Haemophilus influenzae, Staphylococcus aureus, Moraxella (Branhamella) catarrhalis, Neisseria meningitides, and Klebsiella pneumoniae, in a wide variety of samples. As used herein, a “sample” can refer to any type of material of biological origin taken from one or more number of subjects that are suspected of suffering from a respiratory tract infection. The sample can comprise, for example, fluid, tissue or cell. The sample can comprise a biological material taken directly from a subject, or cultured call or tissues, or any fraction or products produced from or derived from biological materials. A sample can be purified, partially purified, unpurified, enriched, or amplified.

The sample can be a biological sample, for example a clinical sample. In some embodiments, the sample is taken from a biological source, such as blood, vagina, urethra, penis, anus, throat, cervix, fermentation broths, cell cultures, and the like. The sample can comprise, for example, fluid and cells from blood and/or respiratory samples. The biological sample can be used (i) directly as obtained from the subject or source, or (ii) following a pre-treatment to modify the character of the sample. Thus, the test sample can be pre-treated prior to use, for example, by disrupting cells or viral particles, preparing liquids from solid materials, diluting viscous fluids, filtering liquids, concentrating liquids, inactivating interfering components, adding reagents, purifying nucleic acids, and the like. Accordingly, a “biological sample” as used herein includes nucleic acids (DNA, RNA or total nucleic acids) extracted from a clinical or biological specimen. Sample preparation can also include using a solution that contains buffers, salts, detergents, and/or the like which are used to prepare the sample for analysis. In some embodiments, the sample is processed before molecular testing. In some embodiments, the sample is analyzed directly, and is not pre-processed prior to testing. The sample can be, for example, a blood sample or respiratory sample. In some embodiments, the sample is a blood sample or respiratory sample from a patient with clinical symptoms of a respiratory tract infection.

Blood or respiratory samples are often infected with multiple organisms. The disclosed primers and probes are tolerant to mixed infections of the blood or respiratory matrix.

In some embodiments, a sample to be tested is processed prior to performing the methods disclosed herein. For example, in some embodiments, the sample can be isolated, concentrated, or subjected to various other processing steps prior to performing the methods disclosed herein. For example, in some embodiments, the sample can be processed to isolate nucleic acids from the sample prior to contacting the sample with the oligonucleotides, as disclosed herein. In some embodiments, the methods disclosed herein are performed on the sample without culturing the sample in vitro. In some embodiments, the methods disclosed herein are performed on the sample without isolating nucleic acids from the sample prior to contacting the sample with oligonucleotides as disclosed herein.

A sample can comprise one or more nucleic acids (e.g., a plurality of nucleic acids). The term “plurality” as used herein can refer two or more. Thus, in some embodiments, a sample includes two or more (e.g., 3 or more, 5 or more, 10 or more, 20 or more, 50 or more, 100 or more, 500 or more, 1,000 or more, or 5,000 or more) nucleic acids (e.g., gDNA, mRNA). A disclosed method can be used as a very sensitive way to detect a target nucleic acid (e.g., nuc gene of S. aureus) present in a sample (e.g., in a complex mixture of nucleic acids such as gDNAs). In some embodiments, the sample includes 5 or more nucleic acids (e.g., 10 or more, 20 or more, 50 or more, 100 or more, 500 or more, 1,000 or more, or 5,000 or more nucleic acids) that differ from one another in sequence. In some embodiments, the sample includes 10 or more, 20 or more, 50 or more, 100 or more, 500 or more, 10³ or more, 5×10³ or more, 10⁴ or more, 5×10⁴ or more, 10⁵ or more, 5×10⁵ or more, 10⁶ or more 5×10⁶ or more, or 10⁷ or more, nucleic acids.

In some embodiments, the sample comprises from 10 to 20, from 20 to 50, from 50 to 100, from 100 to 500, from 500 to 10³, from 10³ to 5×10³, from 5×10³ to 10⁴, from 10⁴ to 5×10⁴, from 5×10⁴ to 10⁵, from 10⁵ to 5×10⁵, from 5×10⁵ to 10⁶, from 10⁶ to 5×10⁶, or from 5×10⁶ to 10⁷, or more than 10⁷, nucleic acids. In some embodiments, the sample comprises from 5 to 10⁷ nucleic acids (e.g., that differ from one another in sequence)(e.g., from 5 to 10⁶, from 5 to 10⁵, from 5 to 50,000, from 5 to 30,000, from 10 to 10⁶, from 10 to 10⁵, from 10 to 50,000, from 10 to 30,000, from 20 to 10⁶, from 20 to 105, from 20 to 50,000, or from 20 to 30,000 nucleic acids, or a number or a range between any two of these values). In some embodiments, the sample includes 20 or more nucleic acids that differ from one another in sequence.

The term “sample” as used herein can mean any sample that includes nucleic acid (e.g., in order to determine whether a target nucleic acid is present among a population of nucleic acids). The sample can be derived from any source, e.g., the sample can be a synthetic combination of purified nucleic acids; the sample can be a cell lysate, an DNA-enriched cell lysate, or nucleic acids isolated and/or purified from a cell lysate. The sample can be from a patient (e.g., for the purpose of diagnosis). The sample can be from permeabilized cells. The sample can be from crosslinked cells. The sample can be in tissue sections. The sample can be from tissues prepared by crosslinking followed by delipidation and adjustment to make a uniform refractive index.

A “sample” can include a target nucleic acid (e.g., nuc gene of S. aureus) and a plurality of non-target nucleic acids. In some embodiments, the target nucleic acid is present in the sample at one copy per 10 non-target nucleic acids, one copy per 20 non-target nucleic acids, one copy per 25 non-target nucleic acids, one copy per 50 non-target nucleic acids, one copy per 100 non-target nucleic acids, one copy per 500 non-target nucleic acids, one copy per 10³ non-target nucleic acids, one copy per 5×10³ non-target nucleic acids, one copy per 10⁴ non-target nucleic acids, one copy per 5×10⁴ non-target nucleic acids, one copy per 10⁵ non-target nucleic acids, one copy per 5×10⁵ non-target nucleic acids, one copy per 10⁶ non-target nucleic acids, less than one copy per 10⁶ non-target nucleic acids, or a number or a range between any two of these values. In some embodiments, the target nucleic acid is present in the sample at from one copy per 10 non-target nucleic acids to 1 copy per 20 non-target nucleic acids, from 1 copy per 20 non-target nucleic acids to 1 copy per 50 non target nucleic acids, from 1 copy per 50 non-target nucleic acids to 1 copy per 100 non-target nucleic acids, from 1 copy per 100 non-target nucleic acids to 1 copy per 500 non-target nucleic acids, from 1 copy per 500 non target nucleic acids to 1 copy per 10³ non-target nucleic acids, from 1 copy per 10³ non-target nucleic acids to 1 copy per 5×10³ non-target nucleic acids, from 1 copy per 5×10³ non-target nucleic acids to 1 copy per 10⁴ non target nucleic acids, from 1 copy per 10⁴ non-target nucleic acids to 1 copy per 10⁵ non-target nucleic acids, from 1 copy per 10⁵ non-target nucleic acids to 1 copy per 10⁶ non-target nucleic acids, or from 1 copy per 10⁶ non target nucleic acids to 1 copy per 10⁷ non-target nucleic acids, or a number or a range between any two of these values.

Suitable samples include but are not limited to saliva, blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising nucleic acids that is obtained from such cells (e.g., a cell lysate or other cell extract comprising nucleic acids).

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available in the art.

In some embodiments, a sample may be an environmental sample, such as water, soil, or a surface such as industrial or medical surface.

Owing to the increased sensitivity of the embodiments disclosed herein, in certain example embodiments, the assays and methods may be run on crude samples or samples where the target molecules to be detected are not further fractionated or purified from the sample.

### Sample Extraction

In sample extractions, cells can be lysed by mechanical shearing with glass beads as described in U.S. Pat. No. 7,494,771, incorporated by reference in its entirety herein, to lyse the target organisms. As disclosed in WO03/008636, such a generic method of cell lysis is efficient for a wide variety of target organisms and specimen matrices. There are also other lysis methods that are designed specifically to target a certain species or group of organisms, or which exploit specific enzymatic or chemical activities. For example, ACP enzyme is commonly used to lyse of Gram-positive organisms (Ezaki et al., J. Clin. Microbiol., 16(5):844-846 (1982); Paule et al., J. Mol. Diagn., 6(3):191-196 (2004); U.S. Pat. No. 3,649,454; all incorporated by reference in their entirety herein) and mycobacteria (U.S. Pat. No. 5,185,242, incorporated by reference in its entirety) but is generally considered to be less efficacious with respect to lysis of Gram-negative species such as E. coli and Pseudomonas aeruginosa (U.S. Pat. No. 3,649,454, incorporated by reference in its entirety).

### Nucleic Acid Testing

The methods described herein can include, for example, nucleic acid testing. For example, the test can include testing for target nucleic acid sequences in a sample. Various forms of nucleic acid testing can be used in the embodiments disclosed herein, including but not limited to, testing that involves nucleic acid amplification. A target nucleic acid (e.g., gDNA, mRNA) can be single-stranded or double-stranded. The source of the target nucleic acid can be any source (e.g., any sample). In some embodiments, the target nucleic acid is a bacterial nucleic acid (e.g., a genomic DNA (gDNA) or an mRNA of a bacterium). As such, the compositions and methods provided herein can be employed for detecting the presence of a bacterial nucleic acid amongst a population of nucleic acids (e.g., in a sample).

Provided herein are compositions and methods for detecting a target nucleic acid (e.g., nuc gene of S. aureus) in a sample that can detect said target nucleic acid with a high degree of sensitivity. In some embodiments, the compositions and methods provided herein can be used to detect a target nucleic acid present in a sample comprising a plurality of nucleic acids (including the target nucleic acid and a plurality of non-target nucleic acids), wherein the target nucleic acid is present at one or more copies per 10⁷ non-target nucleic acids (e.g., one or more copies per 10⁶ non-target nucleic acids, one or more copies per 10⁵ non-target nucleic acids, one or more copies per 10⁴ non-target nucleic acids, one or more copies per 10³ non-target nucleic acids, one or more copies per 10² non-target nucleic acids, one or more copies per 50 non-target nucleic acids, one or more copies per 20 non-target nucleic acids, one or more copies per 10 non-target nucleic acids, or one or more copies per 5 non-target nucleic acids). In some embodiments, the disclosed methods can be used to detect a target nucleic acid present in a sample comprising a plurality of nucleic acids (including the target nucleic acid and a plurality of non-target nucleic acids), wherein the target nucleic acid is present at one or more copies per 10¹⁸ non-target nucleic acids (e.g., one or more copies per 10¹⁵ non-target nucleic acids, one or more copies per 10¹² non-target nucleic acids, one or more copies per 10⁹ non-target nucleic acids, one or more copies per 10⁶ non-target nucleic acids, one or more copies per 10⁵ non-target nucleic acids, one or more copies per 10⁴ non-target nucleic acids, one or more copies per 10³ non-target nucleic acids, one or more copies per 10² non-target nucleic acids, one or more copies per 50 non-target nucleic acids, one or more copies per 20 non-target nucleic acids, one or more copies per 10 non-target nucleic acids, or one or more copies per 5 non-target nucleic acids).

In some embodiments, the threshold of detection, for a disclosed methods of detecting a target nucleic acid (e.g., nuc gene of S. aureus) in a sample, is 10 nM or less. The term “threshold of detection” as used herein can describe the minimal amount of target nucleic acid that must be present in a sample in order for detection to occur. Thus, as an illustrative example, when a threshold of detection is 10 nM, then a signal can be detected when a target nucleic acid is present in the sample at a concentration of 10 nM or more. In some embodiments, the threshold of detection (for detecting the target nucleic acid in a disclosed method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM, or a number or a range between any two of these values) (where the concentration refers to the threshold concentration of target nucleic acid at which the target nucleic acid can be detected). In some embodiments, a disclosed method has a threshold of detection in a range of from 800 fM to 100 pM. In some embodiments, a disclosed method has a threshold of detection in a range of from 1 pM to 10 pM. In some embodiments, a disclosed method has a threshold of detection in a range of from 10 fM to 500 fM, e.g., from 10 fM to 50 fM, from 50 fM to 100 fM, from 100 fM to 250 fM, or from 250 fM to 500 fM, or a number or a range between any two of these values.

In some embodiments, the minimum concentration at which a target nucleic acid (e.g., nuc gene of S. aureus) can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM, or a number or a range between any two of these values). In some embodiments, the minimum concentration at which a target nucleic acid can be detected in a sample is in a range of from 800 fM to 100 pM. In some embodiments, the minimum concentration at which a target nucleic acid can be detected in a sample is in a range of from 1 pM to 10 pM.

In some embodiments, the threshold of detection (for detecting the target nucleic acid in a disclosed method), is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM, or a number or a range between any two of these values) (where the concentration refers to the threshold concentration of target nucleic acid at which the target nucleic acid can be detected). In some embodiments, a disclosed method has a threshold of detection in a range of from 1 aM to 800 aM. In some embodiments, a disclosed method has a threshold of detection in a range of from 50 aM to 1 pM. In some embodiments, a disclosed method has a threshold of detection in a range of from 50 aM to 500 fM.

In some embodiments, the minimum concentration at which a target nucleic acid (e.g., nuc gene of S. aureus) can be detected in a sample is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, or from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM, or a number or a range between any two of these values). In some embodiments, the minimum concentration at which a target nucleic acid can be detected in a sample is in a range of from 1 aM to 500 pM. In some embodiments, the minimum concentration at which a target nucleic acid can be detected in a sample is in a range of from 100 aM to 500 pM. In some embodiments, a composition or method provided herein exhibits an attomolar (aM) sensitivity of detection. In some embodiments, a subject composition or method exhibits a femtomolar (fM) sensitivity of detection. In some embodiments, a subject composition or method exhibits a picomolar (pM) sensitivity of detection. In some embodiments, a subject composition or method exhibits a nanomolar (nM) sensitivity of detection.

As used herein, nucleic acid amplification can refer to any known procedure for obtaining multiple copies of a target nucleic acid sequence or its complement or fragments thereof, using sequence-specific methods. Examples of known amplification methods include, but are not limited to, polymerase chain reaction (PCR), ligase chain reaction (LCR), loop-mediated isothermal amplification (LAMP), strand displacement amplification (SDA) (e.g., multiple displacement amplification (MDA)), replicase-mediated amplification, immuno-amplification, nucleic acid sequence based amplification (NASBA), self-sustained sequence replication (3SR), rolling circle amplification, and transcription-mediated amplification (TMA). See, e.g., Mullis, “Process for Amplifying, Detecting, and/or Cloning Nucleic Acid Sequences,” U.S. Pat. No. 4,683,195; Walker, “Strand Displacement Amplification,” U.S. Pat. No. 5,455,166; Dean et al., “Multiple displacement amplification,” U.S. Pat. No. 6,977,148; Notomi et al., “Process for Synthesizing Nucleic Acid,” U.S. Pat. No. 6,410,278; Landegren et al., U.S. Pat. No. 4,988,617 “Method of detecting a nucleotide change in nucleic acids”; Birkenmeyer, “Amplification of Target Nucleic Acids Using Gap Filling Ligase Chain Reaction,” U.S. Pat. No. 5,427,930; Cashman, “Blocked-Polymerase Polynucleotide Immunoassay Method and Kit,” U.S. Pat. No. 5,849,478; Kacian et al., “Nucleic Acid Sequence Amplification Methods,” U.S. Pat. No. 5,399,491; Malek et al., “Enhanced Nucleic Acid Amplification Process,” U.S. Pat. No. 5,130,238; Lizardi et al., BioTechnology, 6:1197 (1988); Lizardi et al., U.S. Pat. No. 5,854,033 “Rolling circle replication reporter systems.” In some embodiments, two or more of the aforementioned nucleic acid amplification methods can be performed, for example sequentially.

For example, LCR amplification uses at least four separate oligonucleotides to amplify a target and its complementary strand by using multiple cycles of hybridization, ligation, and denaturation (e.g., as described in EP Patent No. 0 320 308). SDA amplifies by using a primer that contains a recognition site for a restriction endonuclease which nicks one strand of a hemimodified DNA duplex that includes the target sequence, followed by amplification in a series of primer extension and strand displacement steps (e.g., as described in U.S. Pat. No. 5,422,252).

PCR is a method well-known in the art for amplification of nucleic acids. PCR involves amplification of a target sequence using two or more extendable sequence-specific oligonucleotide primers that flank the target sequence. The nucleic acid containing the target sequence of interest is subjected to a program of multiple rounds of thermal cycling (denaturation, annealing and extension) in the presence of the primers, a thermostable DNA polymerase (e.g., Taq polymerase) and various dNTPs, resulting in amplification of the target sequence. PCR uses multiple rounds of primer extension reactions in which complementary strands of a defined region of a DNA molecule are simultaneously synthesized by a thermostable DNA polymerase. At the end of each cycle, each newly synthesized DNA molecule acts as a template for the next cycle. During repeated rounds of these reactions, the number of newly synthesized DNA strands increases exponentially such that after 20 to 30 reaction cycles, the initial template DNA will have been replicated several thousand-fold or million-fold. Methods for carrying out different types and modes of PCR are thoroughly described in the literature, for example in “PCR Primer: A Laboratory Manual” Dieffenbach and Dveksler, eds. Cold Spring Harbor Laboratory Press, 1995, and by Mullis et al. in patents (e.g., U.S. Pat. Nos. 4,683,195, 4,683,202 and 4,800,159) and scientific publications (e.g., Mullis et al. 1987, Methods in Enzymology, 155:335-350) where the contents of each reference are hereby incorporated by reference in their entireties.

PCR can generate double-stranded amplification products suitable for post-amplification processing. If desired, amplification products can be detected by visualization with agarose gel electrophoresis, by an enzyme immunoassay format using probe-based colorimetric detection, by fluorescence emission technology, or by other detection means known to one of skill in the art.

A wide variety of PCR methods have been described in many sources, for example, Ausubel et al. (eds.), Current Protocols in Molecular Biology, Section 15, John Wiley & Sons, Inc., New York (1994). Examples of PCR method include, but not limited to, Real-Time PCR, End-Point PCR, Amplified fragment length polymorphism PCR (AFLP-PCR), Alu-PCR, Asymmetric PCR, Colony PCR, DD-PCR, Degenerate PCR, Hot-start PCR, In situ PCR, Inverse PCR Long-PCR, Multiplex PCR, Nested PCR, PCR-ELISA, PCR-RFLP, PCR-single strand conformation polymorphism (PCR-SSCP), quantitative competitive PCR (QC-PCR), rapid amplification of cDNA ends-PCR (RACE-PCR), Random Amplification of Polymorphic DNA-PCR (RAPD-PCR), Real-Time PCR, Repetitive extragenic palindromic-PCR (Rep-PCR), reverse transcriptase PCR (RT-PCR), TAIL-PCR, Touchdown PCR and Vectorette PCR.

Real-time PCR, also called quantitative real time polymerase chain reaction (QRT-PCR), can be used to simultaneously quantify and amplify a specific part of a given nucleic acid molecule. It can be used to determine whether a specific sequence is present in the sample; and if it is present, the number of copies of the sequence that are present. The term “real-time” can refer to periodic monitoring during PCR. Certain systems such as the ABI 7700 and 7900HT Sequence Detection Systems (Applied Biosystems, Foster City, Calif.) conduct monitoring during each thermal cycle at a pre-determined or user-defined point. Real-time analysis of PCR with fluorescence resonance energy transfer (FRET) probes measures fluorescent dye signal changes from cycle-to-cycle, preferably minus any internal control signals. The real-time procedure follows the general pattern of PCR, but the nucleic acid is quantified after each round of amplification. Two examples of method of quantification are the use of fluorescent dyes (e.g., SYBR Green) that intercalate into double-stranded DNA, and modified DNA oligonucleotide probes that fluoresce when hybridized with a complementary DNA. Intercalating agents have a relatively low fluorescence when unbound, and a relatively high fluorescence upon binding to double-stranded nucleic acids. As such, intercalating agents can be used to monitor the accumulation of double strained nucleic acids during a nucleic acid amplification reaction. Examples of such non-specific dyes useful in the embodiments disclosed herein include intercalating agents such as SYBR Green I (Molecular Probes), propidium iodide, ethidium bromide, and the like.

Blood and respiratory samples are often infected with multiple organisms. The disclosed primers and probes are tolerant to mixed infections of the blood and respiratory matrix. Because of the specific target sequences, primers and probes, the methods and compositions disclosed herein can be used to detect the presence/absence or level of Streptococcus pneumoniae, Haemophilus influenzae, Staphylococcus aureus, Moraxella (Branhamella) catarrhalis, Neisseria meningitides, and/or Klebsiella pneumoniae in a sample with high sensitivity, specificity and accuracy.

The primers disclosed herein can be paired with additional PCR systems using a uniform chemistry and thermal PCR profile to provide a panel of assays for the detection of bacterial respiratory pathogens, to improve overall assay sensitivity and robustness.

In some embodiments, multiplex PCR is performed to amplify and detect, e.g., by direct or indirect means, the presence or absence of one or more of Streptococcus pneumoniae, Haemophilus influenzae, Staphylococcus aureus, Moraxella (Branhamella) catarrhalis, Neisseria meningitides, and Klebsiella pneumoniae to allow diagnosis of respiratory tract infections using one or two tests.

Accordingly, some embodiments for the detection and/or identification of S. pneumoniae and N. meningitidis in a sample include the steps of providing a test sample; and contacting the sample with oligonucleotide primers that can specifically hybridize and amplify (1) lytA gene of S. pneumoniae, (2) cpsA gene of S. pneumoniae, (3) sodC gene of N. meningitidis, and (4) crtA gene of N. meningitidis, and oligonucleotide probes that can specifically hybridizes to (1) lytA gene of S. pneumoniae, (2) cpsA gene of S. pneumoniae, (3) sodC gene of N. meningitidis, and (4) crtA gene of N. meningitidis, under standard nucleic acid amplification conditions and/or stringent hybridization conditions. Some embodiments for the detection and/or identification of S. aureus, H. influenzae, M. catarrhalis, and K. pneumoniae in a sample include the steps of providing a test sample; and contacting the sample with oligonucleotide primers that can specifically hybridize and amplify (1) nuc gene of S. aureus, (2) fucK gene of H. influenzae, (3) copB gene of M. catarrhalis, and (4) gltA gene of K. pneumoniae, and oligonucleotide probes that can specifically hybridizes to (1) nuc gene of S. aureus, (2) fucK gene of H. influenzae, (3) copB gene of M. catarrhalis, and (4) gltA gene of K. pneumoniae, under standard nucleic acid amplification conditions and/or stringent hybridization conditions. As described herein, the sample can be contacted with all of the primers and probes at once, or can be contacted with some of the primers and probes first and subsequently contacted by the remainder of the primers and probes.

The oligonucleotide probe can be, for example, between about 10 and about 45 nucleotides in length, and comprises a detectable moiety (e.g., a signal moiety, a detectable label). In some embodiments, the contacting is performed under conditions allowing for the specific hybridization of the primers to the corresponding targeted gene region if the target organism is present in the sample. The presence and/or amount of probe that is specifically bound to the corresponding targeted gene region (if present in the sample being tested) can be determined, wherein bound probe is indicative of the presence of the corresponding target organism in the sample. In some embodiments, the amount of bound probe is used to determine the amount of the corresponding target organism in the sample.

The determining step can be achieved using any methods known to those skilled in the art, including but not limited to, in situ hybridization, following the contacting step. The detection of hybrid duplexes (i.e., of a probe specifically bound to the targeted gene region) can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample. Those of skill in the art will appreciate that wash steps may be employed to wash away excess sample/target nucleic acids or oligonucleotide probe (as well as unbound conjugate, where applicable). Further, standard heterogeneous assay formats are suitable for detecting the hybrids using the labels present on the oligonucleotide primers and probes. Determining the presence or amount of one or more amplicons can comprise contacting said amplicons with a plurality of oligonucleotide probes. At least one of the plurality of oligonucleotide probes comprises a fluorescence emitter moiety and a fluorescence quencher moiety. In some embodiments, determining the presence or amount of one or more amplicons comprises measuring a detectable signal, such as, for example, a detectable signal from a probe.

In some embodiments, determining the presence or amount of one or more amplicons comprises measuring a detectable signal, such as, for example, a detectable signal from a probe (e.g., after cleavage of the probe by the 5′-3′ exonuclease activity of a PCR polymerase (e.g., Taq)). Determining the presence or amount of one or more amplicons can comprise measuring a detectable signal, such as, for example, a detectable signal from a probe. The measuring can in some embodiments be quantitative, e.g., in the sense that the amount of signal detected can be used to determine the amount of target nucleic acid (e.g., nuc gene of S. aureus) present in the sample. The measuring can in some embodiments be qualitative, e.g., in the sense that the presence or absence of detectable signal can indicate the presence or absence of targeted DNA (e.g., virus, SNP, etc.). In some embodiments, a detectable signal will not be present (e.g., above a given threshold level) unless the targeted DNA(s) (e.g., virus, SNP, etc.) is present above a particular threshold concentration. In some embodiments, a disclosed method can be used to determine the amount of a target nucleic acid (e.g., nuc gene of S. aureus) in a sample (e.g., a sample comprising the target nucleic acid and a plurality of non-target nucleic acids). Determining the amount of a target nucleic acid in a sample can comprise comparing the amount of detectable signal generated from a test sample to the amount of detectable signal generated from a reference sample. Determining the amount of a target nucleic acid in a sample can comprise: measuring the detectable signal to generate a test measurement; measuring a detectable signal produced by a reference sample to generate a reference measurement; and comparing the test measurement to the reference measurement to determine an amount of target nucleic acid present in the sample. Determining the amount of a target nucleic acid in a sample can be used to derive the presence and/or amount of an organism comprising said target nucleic acid in a sample.

In some embodiments, a detectable signal is measured is produced by the fluorescence-emitting dye pair of a probe. For example, in some embodiments, a disclosed method includes contacting amplicons with a probe comprising a fluorescence resonance energy transfer (FRET) pair or a quencher/fluor pair, or both. In some embodiments, a disclosed method includes contacting amplicons with a probe comprising a FRET pair. In some embodiments, a disclosed method includes contacting amplicons with a probe comprising a fluor/quencher pair.

Fluorescence-emitting dye pairs comprise a FRET pair or a quencher/fluor pair. In both embodiments of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

In some embodiments (e.g., when the probe includes a FRET pair) the probe produces an amount of detectable signal prior to being cleaved, and the amount of detectable signal that is measured is reduced when the probe is cleaved. In some embodiments, the probe produces a first detectable signal prior to being cleaved (e.g., from a FRET pair) and a second detectable signal when the probe is cleaved (e.g., from a quencher/fluor pair). As such, in some embodiments, the probe comprises a FRET pair and a quencher/fluor pair.

In some embodiments, the probe comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Forster resonance energy transfer”) can refer to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”.

The donor-acceptor pair (a FRET donor moiety and a FRET acceptor moiety) is referred to herein as a “FRET pair” or a “signal FRET pair.” Thus, in some embodiments, a probe includes two signal partners (a signal pair), when one signal partner is a FRET donor moiety and the other signal partner is a FRET acceptor moiety. A probe that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated (e.g., after cleavage of the probe by the 5′-3′ exonuclease activity of a PCR polymerase (e.g., Taq)). FRET donor and acceptor moieties (FRET pairs) will be known to one of ordinary skill in the art and any convenient FRET pair (e.g., any convenient donor and acceptor moiety pair) can be used.

In some embodiments, one signal partner of a signal quenching pair produces a detectable signal and the other signal partner is a quencher moiety that quenches the detectable signal of the first signal partner (e.g., the quencher moiety quenches the signal of the signal moiety such that the signal from the signal moiety is reduced (quenched) when the signal partners are in proximity to one another, e.g., when the signal partners of the signal pair are in close proximity).

For example, in some embodiments, an amount of detectable signal increases when the probe is cleaved. For example, in some embodiments, the signal exhibited by one signal partner (a signal moiety, a fluorescence emitter moiety) is quenched by the other signal partner (a quencher signal moiety, a fluorescence quencher moiety), e.g., when both are present on the same ssDNA molecule prior to cleavage by the 5′-3′ exonuclease activity of a PCR polymerase (e.g., Taq). Such a signal pair is referred to herein as a “quencher/fluor pair”, “quenching pair”, or “signal quenching pair.” For example, in some embodiments, one signal partner (e.g., the first signal partner) is a signal moiety that produces a detectable signal that is quenched by the second signal partner (e.g., a quencher moiety). The signal partners of such a quencher/fluor pair will thus produce a detectable signal when the partners are separated (e.g., after cleavage of the probe by the 5′-3′ exonuclease activity of a PCR polymerase (e.g., Taq)), but the signal will be quenched when the partners are in close proximity (e.g., prior to cleavage of the probe by the 5′-3′ exonuclease activity of a PCR polymerase (e.g., Taq)).

A quencher moiety can quench a signal from the signal moiety (e.g., prior to cleavage of the probe by the 5′-3′ exonuclease activity of a PCR polymerase (e.g., Taq)) to various degrees. In some embodiments, a quencher moiety quenches the signal from the signal moiety where the signal detected in the presence of the quencher moiety (when the signal partners are in proximity to one another) is 95% or less of the signal detected in the absence of the quencher moiety (when the signal partners are separated). For example, in some embodiments, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. In some embodiments, no signal (e.g., above background) is detected in the presence of the quencher moiety.

In some embodiments, the signal detected in the absence of the quencher moiety (when the signal partners are separated) is at least 1.2 fold greater (e.g., at least 1.3 fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater, or a number or a range between any two of these values) than the signal detected in the presence of the quencher moiety (when the signal partners are in proximity to one another).

In some embodiments, the signal moiety is a fluorescent label. In some such embodiments, the quencher moiety quenches the signal (e.g., the light signal) from the fluorescent label (e.g., by absorbing energy in the emission spectra of the label). Thus, when the quencher moiety is not in proximity with the signal moiety, the emission (the signal) from the fluorescent label can be detectable because the signal is not absorbed by the quencher moiety. Any convenient donor acceptor pair (signal moiety/quencher moiety pair) can be used and many suitable pairs are known in the art.

In some embodiments, the quencher moiety absorbs energy from the signal moiety (also referred to herein as a “detectable label” or a “detectable moiety”) and then emits a signal (e.g., light at a different wavelength). Thus, in some embodiments, the quencher moiety is itself a signal moiety (e.g., a signal moiety can be 6-carboxyfluorescein while the quencher moiety can be 6-carboxy-tetramethylrhodamine), and in some such embodiments, the pair could also be a FRET pair. In some embodiments, a quencher moiety is a dark quencher. A dark quencher can absorb excitation energy and dissipate the energy in a different way (e.g., as heat). Thus, a dark quencher has minimal to no fluorescence of its own (does not emit fluorescence).

In some embodiments, cleavage of a probe can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some embodiments, cleavage of a probe can be detected by a color-shift. Such a shift can be expressed as a loss of an amount of signal of one color (wavelength), a gain in the amount of another color, a change in the ration of one color to another, and the like.

There are provided, in some embodiments, methods of detecting S. pneumoniae and N. meningitidis in a sample. In some embodiments, the method comprises: contacting said sample with a plurality of pairs of primers, wherein the plurality of pairs of primers comprises: at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 1-10; at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 16-25; at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 31-39; and at least one pair of primers capable of hybridizing to the crtA gene of N. meningitidis, wherein each primer in said at least one pair of primers comprises any one of the sequences of SEQ ID NOs: 45-54, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 45-54. The method can comprise: generating amplicons of the lytA gene sequence of S. pneumoniae, amplicons of the cpsA gene sequence of S. pneumoniae, amplicons of the sodC gene sequence of N. meningitidis, amplicons of the crtA gene sequence of N. meningitidis from said sample, or any combination thereof, if said sample comprises one or both of S. pneumoniae and N. meningitidis. The method can comprise: determining the presence or amount of one or more amplicons as an indication of the presence of one or both of S. pneumoniae and N. meningitidis in said sample. The method can comprise: contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 60-69 and 130-132, and generating amplicons of the IAC from said sample; and determining the presence or amount of the amplicons of the IAC as an indication of the performance of the assay with the sample. In some embodiments, the sample is contacted with a composition comprising the plurality of pairs of primers and the at least one pair of control primers capable of hybridizing to the IAC.

The type of sample can vary, for example, the sample can be, or derived from, a biological sample or an environmental sample. The environmental sample can be obtained from a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. The biological sample can be obtained from a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof. The biological sample can comprise a blood sample, a respiratory sample, and/or cultures thereof.

The plurality of pairs of primers can, in some embodiments, comprise a first primer comprising the sequence of SEQ ID NOs: 1, 3, 5, 7, or 9, a second primer comprising the sequence of SEQ ID NOs: 2, 4, 6, 8, or 10, a third primer comprising the sequence of SEQ ID NOs: 16, 18, 20, 22, or 24, a fourth primer comprising the sequence of SEQ ID NOs: 17, 19, 21, 23, or 25, a fifth primer comprising the sequence of SEQ ID NOs: 31, 33, 35, 36, or 38, a sixth primer comprising the sequence of SEQ ID NOs: 32, 34, 37, or 39, a seventh primer comprising the sequence of SEQ ID NOs: 45, 47, 49, 51, or 53, and an eighth primer comprising the sequence of SEQ ID NOs: 46, 48, 50, 52, or 54. The plurality of pairs of primers can comprise an ninth primer comprising the sequence of SEQ ID NOs: 60, 62, 64, 66, 68, or 131, and a tenth primer comprising the sequence of SEQ ID NOs: 61, 63, 65, 67, 69, 130, or 132.

In some embodiments, the pair of primers capable of hybridizing to the lytA gene of S. pneumoniae is SEQ ID NOs: 1 and 2, SEQ ID NOs: 3 and 4, SEQ ID NOs: 5 and 6, SEQ ID NOs: 7 and 8, or SEQ ID NOs: 9 and 10; the pair of primers capable of hybridizing to the cpsA gene of S. pneumoniae is SEQ ID NOs: 16 and 17, SEQ ID NOs: 18 and 19, SEQ ID NOs: 20 and 21, SEQ ID NOs: 22 and 23, or SEQ ID NOs: 24 and 25; the pair of primers capable of hybridizing to the sodC gene of N. meningitidis is SEQ ID NOs: 31 and 32, SEQ ID NOs: 33 and 34, SEQ ID NOs: 35 and 32, SEQ ID NOs: 36 and 37, or SEQ ID NOs: 38 and 39; and the pair of primers capable of hybridizing to the crtA gene of N. meningitidisis is SEQ ID NOs: 45 and 46, SEQ ID NOs: 47 and 48, SEQ ID NOs: 49 and 50, SEQ ID NOs: 51 and 52, or SEQ ID NOs: 53 and 54. In some embodiments, the pair of control primers capable of hybridizing to the IAC is SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 61 and 130, or SEQ ID NOs: 131 and 132.

The amplification can be carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA). The PCR can be real-time PCR. The PCR can be quantitative real-time PCR (QRT-PCR). Each primer can comprise exogenous nucleotide sequence.

In some embodiments, determining the presence or amount of one or more amplicons comprises contacting the amplicons with a plurality of oligonucleotide probes, wherein each of the plurality of oligonucleotide probes comprises a sequence selected from the group consisting of SEQ ID NOs: 11-15, 26-30, 40-44, 55-59, 70-74, and 133-134, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 11-15, 26-30, 40-44, 55-59, 70-74, and 133-134. Each of the plurality of oligonucleotide probes can comprise a sequence selected from the group consisting of SEQ ID NOs: 11-15, 26-30, 40-44, 55-59, 70-74, and 133-134. Each of the plurality of oligonucleotide probes can consist of a sequence selected from the group consisting of SEQ ID NOs: 11-15, 26-30, 40-44, 55-59, 70-74, and 133-134. Each probe can be flanked by complementary sequences at the 5′ end and 3′ end. In some embodiments, one of the complementary sequences comprises a fluorescence emitter moiety and the other complementary sequence comprises a fluorescence quencher moiety. In some embodiments, at least one of the plurality of oligonucleotide probes comprises a fluorescence emitter moiety and a fluorescence quencher moiety.

There are provided, in some embodiments, methods of detecting S. aureus, H. influenzae, M. catarrhalis, and K. pneumoniae in a sample. In some embodiments, the method comprises: contacting said sample with a plurality of pairs of primers, wherein the plurality of pairs of primers comprises: at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 75-83; at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 88-95; at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 101-110; and at least one pair of primers capable of hybridizing to the gltA gene of K. pneumoniae, wherein each primer in said at least one pair of primers comprises any one of the sequences of SEQ ID NOs: 116-124, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 116-124. The method can comprise: generating amplicons of the nuc gene sequence of S. aureus, amplicons of the fucK gene sequence of H. influenzae, amplicons of the gene sequence of the copB gene sequence of M. catarrhalis, amplicons of the gltA gene sequence of K. pneumoniae from said sample, or any combination thereof, if said sample comprises one or more of S. aureus, H. influenzae, M catarrhalis, and K. pneumoniae. The method can comprise: determining the presence or amount of one or more amplicons as an indication of the presence of one or more of S. aureus, H. influenzae, M. catarrhalis, and K. pneumoniae in said sample. The method can comprise: contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 60-69 and 130-132, and generating amplicons of the IAC from said sample; and determining the presence or amount the amplicons of the IAC as an indication of the performance of the assay with the sample. In some embodiments, the sample is contacted with a composition comprising the plurality of pairs of primers and the at least one pair of control primers capable of hybridizing to the IAC.

The plurality of pairs of primers can comprise a first primer comprising the sequence of SEQ ID NOs: 75, 77, 79, 81, or 83, a second primer comprising the sequence of SEQ ID NOs: 76, 78, 80, or 82, a third primer comprising the sequence of SEQ ID NOs: 88, 90, 92, 93, or 94, a fourth primer comprising the sequence of SEQ ID NOs: 89, 91, or 95, a fifth primer comprising the sequence of SEQ ID NOs: 101, 103, 105, 107, or 109, a sixth primer comprising the sequence of SEQ ID NOs: 102, 104, 106, 108, or 110, a seventh primer comprising the sequence of SEQ ID NOs: 116, 118, 120, or 122, and an eighth primer comprising the sequence of SEQ ID NOs: 117, 119, 121, 123, or 124. The plurality of pairs of primers can comprise an ninth primer comprising the sequence of SEQ ID NOs: 60, 62, 64, 66, 68, or 131, and a tenth primer comprising the sequence of SEQ ID NOs: 61, 63, 65, 67, 69, 130, or 132.

In some embodiments, the pair of primers capable of hybridizing to the nuc gene of S. aureus is SEQ ID NOs: 75 and 76, SEQ ID NOs: 77 and 78, SEQ ID NOs: 79 and 80, SEQ ID NOs: 81 and 82, or SEQ ID NOs: 83 and 78; the pair of primers capable of hybridizing to the fucK gene of H. influenzae is SEQ ID NOs: 88 and 89, SEQ ID NOs: 90 and 91, SEQ ID NOs: 92 and 89, SEQ ID NOs: 93 and 89, or SEQ ID NOs: 94 and 95; the pair of primers capable of hybridizing to the copB gene of M. catarrhalis is SEQ ID NOs: 101 and 102, SEQ ID NOs: 103 and 104, SEQ ID NOs: 105 and 106, SEQ ID NOs: 107 and 108, or SEQ ID NOs: 109 and 110; and the pair of primers capable of hybridizing to the gltA gene of K. pneumoniae is SEQ ID NOs: 116 and 117, SEQ ID NOs: 118 and 119, SEQ ID NOs: 120 and 121, SEQ ID NOs: 122 and 123, or SEQ ID NOs: 116 and 124. In some embodiments, the pair of control primers capable of hybridizing to the IAC is SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 61 and 130, or SEQ ID NOs: 131 and 132.

In some embodiments, determining the presence or amount of one or more amplicons comprises contacting the amplicons with a plurality of oligonucleotide probes, wherein each of the plurality of oligonucleotide probes comprises a sequence selected from the group consisting of SEQ ID NOs: 84-87, 96-100, 111-115, 125-129, 70-74, and 133-134, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 84-87, 96-100, 111-115, 125-129, 70-74, and 133-134. Each of the plurality of oligonucleotide probes can comprise a sequence selected from the group consisting of SEQ ID NOs: 84-87, 96-100, 111-115, 125-129, 70-74, and 133-134. Each of the plurality of oligonucleotide probes can consist of a sequence selected from the group consisting of SEQ ID NOs: 84-87, 96-100, 111-115, 125-129, 70-74, and 133-134. Each probe can be flanked by complementary sequences at the 5′ end and 3′ end. In some embodiments, one of the complementary sequences comprises a fluorescence emitter moiety and the other complementary sequence comprises a fluorescence quencher moiety. At least one of the plurality of oligonucleotide probes can comprise a fluorescence emitter moiety and a fluorescence quencher moiety.

As described herein, the amplification can be carried out by real-time PCR, for example, quantitative real-time PCR (QRT-PCR). The primers suitable for use in the methods and compositions described herein can comprise exogenous nucleotide sequence which allows post-amplification manipulation of amplification products without a significant effect on amplification itself. In some embodiments, the primer and/or probe can be flanked by complementary sequences comprising a fluorophore at the 5′ end, and a fluorescence quencher at the 3′ end.

Any of the oligonucleotide probes disclosed herein can comprise a fluorescence emitter moiety, a fluorescence quencher moiety, or both.

The methods disclosed herein are amendable to automation, thereby providing a high-throughput option for the detection and/or quantification of S. pneumoniae and N. meningitidis and/or for the detection and/or quantification of S. aureus, H. influenzae, M. catarrhalis, and K. pneumoniae in a sample. Various multiplex PCR platforms, e.g., BD MAX™, Viper™, or Viper™ LT platforms, can be used to perform one or more steps of the disclosed methods. The methods can be performed in a multiplex fashion. For example, the nucleic acid amplification and/or detection, in some embodiments, comprise performing multiplex PCR.

### EXAMPLES

The following examples are provided to demonstrate particular situations and settings in which this technology may be applied and are not intended to restrict the scope of the invention and the claims included in this disclosure.

### Example 1

Detection of S. pneumoniae and N. meningitidis and Detection of S. aureus, H. influenzae, M. catarrhalis, and K. pneumoniae

The study described in this example describes an implementation case of the compositions and methods provided herein on a BD MAX fully automated system. The compositions and methods disclosed herein can also be implemented on other real-time PCR instruments, such as, for example, ABI 7500.

Materials and Methods

A total of 28 strains were used for the validation of the multiplex PCR, and these are presented in Table 8. These isolates included 15 Gram-negative bacterial strains and 13 Gram-positive bacterial strains. The six control strains used in this study were Streptococcus pneumoniae ATCC 49619, Neisseria meningitides ATCC 13090, Moraxella catarrhalis ATCC 25238, Klebsiella pneumoniae ATCC 13439 and Haemophilus influenzae ATCC 49247. EDTA blood was obtained from healthy human volunteers.

TABLE 8
Bacterial Strains Used for Validation of Multiplex PCR

| | | Multiplex PCR Detection | | | | | | | |
|---|---|---|---|---|---|---|---|---|---|
| | Source | Sp- | Sp- | Nm- | Nm- | Sa- | H.flu- | Mc- | Kp- |
| Species | Strain (no.) | lytA | cpsA | sodC | ctrA | nuc | fucK | copB | gltA |
| Gram-negative | | | | | | | | | |
| aerobic bacteria | | | | | | | | | |
| Enterobacter | NCTC | − | − | − | − | − | − | − | − |
| cloacae | 13922 | | | | | | | | |
| Proteus vulgaris | ATCC | − | − | − | − | − | − | − | − |
| | 13315 | | | | | | | | |
| Acinetobacter | NCTC | − | − | − | − | − | − | − | − |
| baumannii | 13302 | | | | | | | | |
| Klebsiella oxytoca | ATCC | − | − | − | − | − | − | − | − |
| | 13182 | | | | | | | | |
| Pseudomonas | ATCC | − | − | − | − | − | − | − | − |
| aeruginosa | 27853 | | | | | | | | |
| Serratia | NCTC | − | − | − | − | − | − | − | − |
| marcescens | 13920 | | | | | | | | |
| Escherichia coli | ATCC | − | − | − | − | − | − | − | − |
| | 25922 | | | | | | | | |
| Listeria | ATCC | − | − | − | − | − | − | − | − |
| monocytogenes | 19111 | | | | | | | | |
| Klebsiella | NCTC | − | − | − | − | − | − | − | + |
| pneumoniae | 13438 | | | | | | | | |
| | NCTC | − | − | − | − | − | − | − | + |
| | 13439 | | | | | | | | |
| | ATCC | − | − | − | − | − | − | − | + |
| | 700603 | | | | | | | | |
| | Clinical | − | − | − | − | − | − | − | + |
| | isolate | | | | | | | | |
| | AH10067 | | | | | | | | |
| Haemophilus | ATCC | − | − | − | − | − | + | − | − |
| influenzae | 49247 | | | | | | | | |
| Neisseria | ATCC | − | − | + | + | − | − | − | − |
| meningitidis | 13090 | | | | | | | | |
| Moraxella | ATCC | − | − | − | − | − | − | + | − |
| catarrhalis | 25238 | | | | | | | | |
| Gram-positive | | | | | | | | | |
| bacteria | | | | | | | | | |
| Enterococcus | ATCC | − | − | − | − | − | − | − | − |
| faecium | 2278 | | | | | | | | |
| Enterococcus | ATCC | − | − | − | − | − | − | − | − |
| faecalis | 29212 | | | | | | | | |
| Staphylococcus | NCTC | − | − | − | − | − | − | − | − |
| epidermidis | 13924 | | | | | | | | |
| Staphylococcus | ATCC | − | − | − | − | + | − | − | − |
| aureus | 29213 | | | | | | | | |
| | Clinical | − | − | − | − | + | − | − | − |
| | isolate | | | | | | | | |
| | BJ-2 | | | | | | | | |
| | ATCC | − | − | − | − | + | − | − | − |
| | 43300 | | | | | | | | |
| Streptococcus | ATCC | + | + | − | − | − | − | − | − |
| pneumoniae | 49619 | | | | | | | | |
| | ATCC | + | + | − | − | − | − | − | − |
| | 6305 | | | | | | | | |
| | Clinical | + | + | − | − | − | − | − | − |
| | isolate | | | | | | | | |
| | YLL149027 | | | | | | | | |
| | Clinical | + | + | − | − | − | − | − | − |
| | isolate | | | | | | | | |
| | CWSP-8 | | | | | | | | |
| | Clinical | + | + | − | − | − | − | − | − |
| | isolate | | | | | | | | |
| | QZ-9218 | | | | | | | | |
| Streptococcus | ATCC | − | − | − | − | − | − | − | − |
| pyogenes | 19615 | | | | | | | | |
| Streptococcus | ATCC | − | − | − | − | − | − | − | − |
| agalactiae | 13813 | | | | | | | | |

A BD MAX™ ExK™ TNA-2 Extraction Kit, 5× qPCR Mastermix, and Lysis Buffer were employed in this study.

All the target gene sequences were based on alignments of available sequences deposited in the nr database of NCBI (https://www.ncbi.nlm.nih.gov/nucleotide/). All primers and probes were designed using Beacon Designer V8.20, and all were synthesized by Sangon Biotech (Shanghai, China). The NCBI BLASTn was used to check the in silico specificity and sensitivity.

For DNA extraction from blood sample, 1 ml EDTA blood samples (spiked with bacteria) were pretreated with an optimized lysis buffer and then added into the BD MAX sample buffer tube. DNA automated extraction on BD MAX using BD MAX ExK TNA-2 Extraction Kit was conducted following kit instructions.

The multiplex PCR reaction comprising the primer/probe combinations disclosed herein was carried out in a total volume of 12.5 μl, comprising 5 μl of Multiplex PCR master mix (commercial), 2.5 μl of nuclease-free water, 0.3 μM final concentration of the primers disclosed herein, and 0.2 μM final concentration of hybridization probes disclosed herein. The conical tubes containing 12.5 μl mixture were snapped into the BD MAX TNA extraction strip. The final PCR reaction mixture was prepared by BD MAX by automatically adding 12.5 μl purified DNA prepared as described above into the conical tube and mixed.

The PCR thermocycling profile was as follows: 95° C. denaturation for 5 min; and 95° C. denaturation 15s, 60° C. annealing and extension 43s, 40 cycles.

Amplification Efficiency Testing

10 ul the six above-mentioned control strain culture suspensions was spiked into 1 ml negative EDTA blood specimen and pretreated before extraction. These were tested at six different bacterial concentrations in duplicate per run starting from McFarland standard of 2.5, for six independent runs. Colony counts were performed using the standard plate counting procedure (Tables 9-11). Robust amplification efficiencies were observed (Table 12) using the primer/probe combinations provided herein for the two assays (Table 13).

TABLE 9
Colony Counting Results

| | Colony Counting Result (CFU/ml) | | | | | |
|---|---|---|---|---|---|---|
| Sample | Original Conc. | Dilution-1 | Dilution-2 | Dilution-3 | Dilution-4 | Dilution-5 |
| SP-49619 | 1.65E+08 | 1.65E+07 | 1.65E+06 | 1.65E+05 | 1.65E+04 | 1.65E+03 |
| NM13090 | 3.32E+08 | 3.32E+07 | 3.32E+06 | 3.32E+05 | 3.32E+04 | 3.32E+03 |
| H.flu- | 5.06E+08 | 5.06E+07 | 5.06E+06 | 5.06E+05 | 5.06E+04 | 5.06E+03 |
| 49247 | | | | | | |
| MC-25238 | 2.00E+08 | 2.00E+07 | 2.00E+06 | 2.00E+05 | 2.00E+04 | 2.00E+03 |
| KP-13439 | 4.20E+08 | 4.20E+07 | 4.20E+06 | 4.20E+05 | 4.20E+04 | 4.20E+03 |

TABLE 10
Colony Counting Results

| | CFU in SBT CFU/ml) | | | | | |
|---|---|---|---|---|---|---|
| Sample | Original Conc. | Dilution-1 | Dilution-2 | Dilution-3 | Dilution-4 | Dilution-5 |
| SP-49619 | 1.10E+06 | 1.10E+05 | 1.10E+04 | 1.10E+03 | 1.10E+02 | 1.10E+01 |
| NM13090 | 2.21E+06 | 2.21E+05 | 2.21E+04 | 2.21E+03 | 2.21E+02 | 2.21E+01 |
| H.flu- | 3.37E+06 | 3.37E+05 | 3.37E+04 | 3.37E+03 | 3.37E+02 | 3.37E+01 |
| 49247 | | | | | | |
| MC-25238 | 1.33E+06 | 1.33E+05 | 1.33E+04 | 1.33E+03 | 1.33E+02 | 1.33E+01 |
| KP-13439 | 2.80E+06 | 2.80E+05 | 2.80E+04 | 2.80E+03 | 2.80E+02 | 2.80E+01 |

TABLE 11
Colony Counting Results

| | CFU in Blood Sample (CFU/ml) | | | | | |
|---|---|---|---|---|---|---|
| Sample | Original Conc. | Dilution-1 | Dilution-2 | Dilution-3 | Dilution-4 | Dilution-5 |
| SP-49619 | 1.65E+06 | 1.65E+05 | 1.65E+04 | 1.65E+03 | 1.65E+02 | 1.65E+01 |
| NM13090 | 3.32E+06 | 3.32E+05 | 3.32E+04 | 3.32E+03 | 3.32E+02 | 3.32E+01 |
| H.flu- | 5.06E+06 | 5.06E+05 | 5.06E+04 | 5.06E+03 | 5.06E+02 | 5.06E+01 |
| 49247 | | | | | | |
| MC-25238 | 2.00E+06 | 2.00E+05 | 2.00E+04 | 2.00E+03 | 2.00E+02 | 2.00E+01 |
| KP-13439 | 4.20E+06 | 4.20E+05 | 4.20E+04 | 4.20E+03 | 4.20E+02 | 4.20E+01 |

TABLE 12
Amplification Efficiency Testing Results

| | | | | Amplification |
|---|---|---|---|---|
| Method | Strain spiked in blood | Target | R² | Efficiency |
| Assay 1 | Streptococcus pneumoniae | lytA | 0.99 | 123.38% |
| | ATCC 49619 | CpsA | 0.99 | 125.42% |
| | Neisseria meningitides | SodC | 1.00 | 114.12% |
| | ATCC 13090 | CtrA | 1.00 | 108.44% |
| Assay 2 | Haemophilus influenzae | fuck | 0.98 | 90.06% |
| | ATCC 49247 | | | |
| | Moraxella catarrhalis | copB | 1.00 | 111.56% |
| | ATCC 25238 | | | |
| | Klebsiella pneumoniae | gltA | 1.00 | 110.17% |
| | ATCC 13439 | | | |

TABLE 13
Primers and Probes

| Primer/ | Reaction | | |
|---|---|---|---|
| probe ID | conc. (nM) | Fluorescence Label | Assay |
| LytA-FP | 300 | | 1 |
| LytA-RP | 300 | | 1 |
| LytA-probe | 100 | 5′6-FAM, 3′BHQ1 | 1 |
| CpsA-FP | 300 | | 1 |
| CpsA-RP | 300 | | 1 |
| CpsA-probe | 100 | 5′VIC, 3′BHQ1 | 1 |
| sodC-FP | 300 | | 1 |
| sodC-RP | 300 | | 1 |
| sodC-probe | 100 | 5′ROX, 3′BHQ2 | 1 |
| ctrA-FP | 300 | | 1 |
| ctrA-RP | 300 | | 1 |
| ctrA-probe | 100 | 5′CY5, 3′BHQ-3 | 1 |
| IAC-FP | 300 | | 1/2 |
| IAC-RP | 300 | | 1/2 |
| IAC-probe | 100 | 5′CY5.5, 3′BHQ3 | 1/2 |
| nuc-FP | 300 | | 2 |
| nuc-RP | 300 | | 2 |
| nuc-Probe | 100 | 5′ FAM, BHQ1 on “T”, 3′ SpC6 | 2 |
| fucK-FP | 300 | | 2 |
| fucK-RP | 300 | | 2 |
| fucK-probe | 100 | 5′VIC, 3′BHQ1 | 2 |
| copB-FP | 300 | | 2 |
| copB-RP | 300 | | 2 |
| copB-Probe | 100 | 5′ROX, 3′BHQ2 | 2 |
| gltA-FP | 300 | | 2 |
| gltA-RP | 300 | | 2 |
| gltA-probe | 100 | 5′CY5, 3′BHQ-3 | 2 |

Analytical Sensitivity Testing

For estimation of the limit of detection of the optimized multiplex PCR assays provided herein (Table 13), we spiked 10 ul the six above-mentioned control strain culture suspensions with 1 ml negative EDTA blood specimen and pretreated before extraction. These were tested at six different bacterial concentrations in duplicate per run starting from McFarland standard of 2.5, for six independent runs. Colony counts were performed using the standard plate counting procedure (Tables 14-16). The highest 10-fold dilution for which a threshold cycle CT value was observed was diluted further in a series of three 2-fold dilutions (1:2, 1:4, and 1:8) to find the lowest concentration at which a CT value was detected. The lowest concentration that produced a CT value was tested in 6 replicates to determine the limit of detection (LoD) of the assay (Table 17). Robust analytical sensitivity was observed for each target using the primer/probe combinations provided herein.

TABLE 14
Colony Counting Results

| | Colony counts Result (CFU/ml) | | | |
|---|---|---|---|---|
| Sample | Original Conc. | Dilution-1 | Dilution-2 | Dilution-3 |
| SP-49619 | 1.65E+04 | 8.25E+03 | 4.13E+03 | 2.06E+03 |
| NM13090 | 3.32E+04 | 1.66E+04 | 8.30E+03 | 4.15E+03 |
| H. flu- | 5.06E+04 | 2.53E+04 | 1.27E+04 | 6.33E+03 |
| 49247 | | | | |
| MC-25238 | 2.00E+04 | 1.00E+04 | 5.00E+03 | 2.50E+03 |
| KP-13439 | 4.20E+04 | 2.10E+04 | 1.05E+04 | 5.25E+03 |

TABLE 15
Colony Counting Results

| | CFU in SBT (CFU/ml) | | | |
|---|---|---|---|---|
| Sample | Original Conc. | Dilution-1 | Dilution-2 | Dilution-3 |
| SP-49619 | 110.00 | 55.00 | 27.50 | 13.75 |
| NM13090 | 221.33 | 110.67 | 55.33 | 27.67 |
| H. flu-49247 | 337.33 | 168.67 | 84.33 | 42.17 |
| MC-25238 | 133.33 | 66.67 | 33.33 | 16.67 |
| KP-13439 | 280.00 | 140.00 | 70.00 | 35.00 |

TABLE 16
Colony Counting Results

| | CFU in Blood Sample (CFU/ml) | | | |
|---|---|---|---|---|
| Sample | Original Conc. | Dilution-1 | Dilution-2 | Dilution-3 |
| SP-49619 | 165.00 | 82.50 | 41.25 | 20.63 |
| NM13090 | 332.00 | 166.00 | 83.00 | 41.50 |
| H. flu-49247 | 506.00 | 253.00 | 126.50 | 63.25 |
| MC-25238 | 200.00 | 100.00 | 50.00 | 25.00 |
| KP-13439 | 420.00 | 210.00 | 105.00 | 52.50 |

TABLE 17
Analytical Sensitivity Testing Results

| | LoD (CFU/mL | LoD (CFU/mL |
|---|---|---|
| | in PCR) | in Blood) |
| Streptococcus pneumoniae ATCC | 11(83.3%) | 82.5(83.3%) |
| 49619 | | |
| Neisseria meningitides ATCC | 11.07(83.3%) | 83(83.3%) |
| 13090 | | |
| Moraxella catarrhalis ATCC | 6.67(83.3%) | 50(83.3%) |
| 25238 | | |
| Klebsiella pneumoniae ATCC | 28(100%) | 210(100%) |
| 13439 | | |
| Haemophilus influenzae ATCC | 33.73(100%) | 253(100%) |
| 49247 | | |

Analytical Specificity Testing

Analytical specificity was measured by testing DNA extracted from the panel of positive- and negative-control isolates shown in Table 8. This panel consisted of 28 control isolates that were either closely related to the target species or represented a wide range of pathogenic isolates which are commonly found in respiratory samples of fever patients and identified using the cultural method. As seen in Tables 18-21, the assay correctly detected all of the target isolates (S. pneumoniae, H. influenzae, S. aureus, M. catarrhalis, N. meningitides, and K. pneumoniae), and there was no cross-reaction with the non-target isolates listed in Table 8 using the primer/probe combinations provided herein.

TABLE 18
Analytical Specificity Results for Gram-negative Aerobic Bacteria

| | Strain tested | Specificity |
|---|---|---|
| Species | n = 15 | (100%) |
| Gram-negative aerobic | | |
| bacteria | | |
| Enterobacter cloacae | NCTC 13922 | 100 |
| Proteus vulgaris | ATCC 13315 | 100 |
| Acinetobacter baumannii | NCTC 13302 | 100 |
| Klebsiella oxytoca | ATCC 13182 | 100 |
| Pseudomonas aeruginosa | ATCC 27853 | 100 |
| Serratia marcescens | NCTC 13920 | 100 |
| Escherichia coli | ATCC 25922 | 100 |
| Listeria monocytogenes | ATCC 19111 | 100 |
| Klebsiella pneumoniae | NCTC 13438 | 100 |
| | NCTC 13439 | 100 |
| | ATCC 700603 | 100 |
| | Clinical isolate AH10067 | 100 |
| Haemophilus influenzae | ATCC 49247 | 100 |
| Neisseria meningitidis | ATCC 13090 | 100 |
| Moraxella catarrhalis | ATCC 25238 | 100 |

TABLE 19
Analytical Specificity Results for Gram-positive Aerobic Bacteria

| | Strain tested | Specificity |
|---|---|---|
| Species | n = 13 | (100%) |
| Gram-positive bacteria | | 100 |
| Enterococcus faecium | ATCC 2278 | 100 |
| Enterococcus faecalis | ATCC 29212 | 100 |
| Staphylococcus epidermidis | NCTC 13924 | 100 |
| Staphylococcus aureus | ATCC 29213 | 100 |
| | Clinical isolate BJ-2 | 100 |
| | ATCC 43300 | 100 |
| Streptococcus pneumoniae | ATCC 49619 | 100 |
| | ATCC 6305 | 100 |
| | Clinical isolate YLL149027 | 100 |
| | Clinical isolate CWSP-8 | 100 |
| | Clinical isolate QZ-9218 | 100 |
| Streptococcus pyogenes | ATCC 19615 | 100 |
| Streptococcus agalactiae | ATCC 13813 | 100 |

TABLE 20
Analytical Specificity Testing Results for Assay 1

| | NM- | NM- | SP- | SP- | |
|---|---|---|---|---|---|
| Accession Number | SodC | CtrA | lytA | CpsA | SPC |
| Enterobacter cloacae-13922 | −1 | −1 | −1 | −1 | 29.5 |
| Enterococcus Faecium-2278 | −1 | −1 | −1 | −1 | 27.8 |
| Klebsiella oxytoca-13182 | −1 | −1 | −1 | −1 | 26.7 |
| Serratia marcescens-13920 | −1 | −1 | −1 | −1 | 28.5 |
| Acinetobacter baumannii-13302 | −1 | −1 | −1 | −1 | 29.5 |
| Proteus vulgaris-13315 | −1 | −1 | −1 | −1 | 28.8 |
| Streptococcus pyogenes-19615 | −1 | −1 | −1 | −1 | 29.5 |
| Streptococcus agalactiae-13813 | −1 | −1 | −1 | −1 | 28.2 |
| Staphylococcus aureus-29213 | −1 | −1 | −1 | −1 | 29.4 |
| Pseudomonas aeruginosa-27853 | −1 | −1 | −1 | −1 | 29.3 |
| Staphylococcus epidermidis-13924 | −1 | −1 | −1 | −1 | 29.5 |
| Klebsiella pneumoniae-13439 | −1 | −1 | −1 | −1 | 24.5 |
| Enterococcus faecalis-29212 | −1 | −1 | −1 | −1 | 26.7 |
| Streptococcus pneumoniae-49619 | −1 | −1 | 22.3 | 22.4 | 28.4 |
| Listeria monocytogenes-19111 | −1 | −1 | −1 | −1 | 30 |
| Streptococcus pneumoniae-6305 | 26.4 | 27.2 | −1 | −1 | 26.7 |
| Streptococcus pneumoniae | 30 | 29.6 | −1 | −1 | 31.3 |
| YLL149027 | | | | | |
| Streptococcus pneumoniae CWSP- | 27.4 | 28.1 | −1 | −1 | 28.7 |
| 8 | | | | | |
| Streptococcus pneumoniae QZ- | 27.3 | 28 | −1 | −1 | 26.5 |
| 9218 | | | | | |
| Neisseria meningitidis-13090 | 17.9 | 17.1 | −1 | −1 | 28 |
| Haemophilus influenzae-49247 | −1 | −1 | −1 | −1 | 29.3 |
| Staphylococcus aureus-BJ-2 | −1 | −1 | −1 | −1 | 30.1 |
| Moraxella catarrhalis-25238 | −1 | −1 | −1 | −1 | 29.8 |
| Klebsiella pneumoniae-13438 | −1 | −1 | −1 | −1 | 29.8 |
| Klebsiella pneumoniae-700603 | −1 | −1 | −1 | −1 | 29.7 |
| Staphylococcus aureus-43300 | −1 | −1 | −1 | −1 | 29.6 |
| Klebsiella pneumoniae- | −1 | −1 | −1 | −1 | 29.4 |
| KPAH10067 | | | | | |
| Escherichia coli-25922 | −1 | −1 | −1 | −1 | 29.4 |

TABLE 21
Analytical Specificity Testing Results for Assay 2

| | | H. | | | |
|---|---|---|---|---|---|
| | SA- | FLU- | MC- | KP- | |
| Sample number | nuc | fuck | copB | gltA | SPC |
| Enterobacter cloacae 13922 | −1 | −1 | −1 | −1 | 29.4 |
| Enterococcus faecium-2278 | −1 | −1 | −1 | −1 | 29.1 |
| Klebsiella oxytoca-13182 | −1 | −1 | −1 | −1 | 28.9 |
| Serratia marcescens-13920 | −1 | −1 | −1 | −1 | 28.7 |
| Acinetobacter baumannii-13302 | −1 | −1 | −1 | −1 | 29.2 |
| Proteus vulgaris-13315 | −1 | −1 | −1 | −1 | 29.4 |
| Streptococcus pyogenes-19615 | −1 | −1 | −1 | −1 | 28.9 |
| Streptococcus agalactiae-13813 | −1 | −1 | −1 | −1 | 29.1 |
| Staphylococcus aureus-29213 | 24.3 | −1 | −1 | −1 | 28.2 |
| Pseudomonas aeruginosa-27853 | −1 | −1 | −1 | −1 | −1 |
| Staphylococcus epidermidis-13924 | −1 | −1 | −1 | −1 | 29 |
| Klebsiella pneumoniae-13439 | −1 | −1 | −1 | 21.6 | 28 |
| Enterococcus faecalis-29212 | −1 | −1 | −1 | −1 | 30.1 |
| Streptococcus pneumoniae-49619 | −1 | −1 | −1 | −1 | 29.5 |
| Listeria monocytogenes-19111 | −1 | −1 | −1 | −1 | 30.1 |
| Streptococcus pneumoniae-6305 | −1 | −1 | −1 | −1 | 29.8 |
| Streptococcus pneumoniae | −1 | −1 | −1 | −1 | 29.7 |
| YLL149027 | | | | | |
| Streptococcus pneumoniae | −1 | −1 | −1 | −1 | 29.9 |
| CWSP-8 | | | | | |
| Streptococcus pneumoniae | −1 | −1 | −1 | −1 | 29.6 |
| QZ-9218 | | | | | |
| Neisseria meningitidis-13090 | −1 | −1 | 25.9 | 26 | 26.5 |
| Haemophilus influenzae-49247 | −1 | 29 | −1 | −1 | 29.2 |
| Staphylococcus aureus-BJ-2 | 27.3 | −1 | −1 | −1 | 29 |
| Moraxella catarrhalis-25238 | −1 | −1 | 28.1 | −1 | 28.4 |
| Klebsiella pneumoniae-13438 | −1 | −1 | −1 | 27.4 | 27.6 |
| Klebsiella pneumoniae-700603 | −1 | −1 | −1 | 27 | 27.7 |
| Staphylococcus aureus-43300 | 26.1 | −1 | −1 | −1 | 29 |
| Klebsiella pneumoniae - | −1 | −1 | −1 | 23.5 | 28.6 |
| KPAH10067 | | | | | |
| Escherichia coli-25922 | −1 | −1 | −1 | −1 | 29.5 |

Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of detecting S. pneumoniae and N. meningitidis in a sample, comprising:
contacting said sample with a plurality of pairs of primers, wherein the plurality of pairs of primers comprises:
at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity to any one of the sequences of SEQ ID NOs: 1-10;
at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity to any one of the sequences of SEQ ID NOs: 16-25;
at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity to any one of the sequences of SEQ ID NOs: 31-39; and
at least one pair of primers capable of hybridizing to the crtA gene of N. meningitidis, wherein each primer in said at least one pair of primers comprises any one of the sequences of SEQ ID NOs: 45-54, or a sequence that exhibits at least about 85% identity to any one of the sequences of SEQ ID NOs: 45-54;
generating amplicons of the lytA gene sequence of S. pneumoniae, amplicons of the cpsA gene sequence of S. pneumoniae, amplicons of the sodC gene sequence of N. meningitidis, amplicons of the crtA gene sequence of N. meningitidis from said sample, or any combination thereof, if said sample comprises one or both of S. pneumoniae and N. meningitidis; and
determining the presence or amount of one or more amplicons as an indication of the presence of one or both of S. pneumoniae and N. meningitidis in said sample.

2. The method of claim 1, further comprising contacting the sample with at least one pair of control primers capable of hybridizing to an internal amplification control (IAC) added to the sample, wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132, and
generating amplicons of the IAC from said sample; and
determining the presence or amount of the amplicons of the IAC as an indication of the performance of the assay with the sample.

3. The method of claim 2, wherein the sample is contacted with a composition comprising the plurality of pairs of primers and the at least one pair of control primers capable of hybridizing to the IAC.

4. The method of claim 1, wherein the sample is a biological sample or an environmental sample.

5.-6. (canceled)

7. The method of claim 4, wherein the biological sample comprises a blood sample, a respiratory sample, and/or cultures thereof.

8. The method of claim 1, wherein the plurality of pairs of primers comprises a first primer comprising the sequence of SEQ ID NOs: 1, 3, 5, 7, or 9, a second primer comprising the sequence of SEQ ID NOs: 2, 4, 6, 8, or 10, a third primer comprising the sequence of SEQ ID NOs: 16, 18, 20, 22, or 24, a fourth primer comprising the sequence of SEQ ID NOs: 17, 19, 21, 23, or 25, a fifth primer comprising the sequence of SEQ ID NOs: 31, 33, 35, 36, or 38, a sixth primer comprising the sequence of SEQ ID NOs: 32, 34, 37, or 39, a seventh primer comprising the sequence of SEQ ID NOs: 45, 47, 49, 51, or 53, and an eighth primer comprising the sequence of SEQ ID NOs: 46, 48, 50, 52, or 54.

9. The method of claim 1, wherein the plurality of pairs of primers comprises a ninth primer comprising the sequence of SEQ ID NOs: 60, 62, 64, 66, 68, or 131, and a tenth primer comprising the sequence of SEQ ID NOs: 61, 63, 65, 67, 69, 130, or 132.

10. The method of claim 1, wherein
the pair of primers capable of hybridizing to the lytA gene of S. pneumoniae is SEQ ID NOs: 1 and 2, SEQ ID NOs: 3 and 4, SEQ ID NOs: 5 and 6, SEQ ID NOs: 7 and 8, or SEQ ID NOs: 9 and 10;
the pair of primers capable of hybridizing to the cpsA gene of S. pneumoniae is SEQ ID NOs: 16 and 17, SEQ ID NOs: 18 and 19, SEQ ID NOs: 20 and 21, SEQ ID NOs: 22 and 23, or SEQ ID NOs: 24 and 25;
the pair of primers capable of hybridizing to the sodC gene of N. meningitidis is SEQ ID NOs: 31 and 32, SEQ ID NOs: 33 and 34, SEQ ID NOs: 35 and 32, SEQ ID NOs: 36 and 37, or SEQ ID NOs: 38 and 39; and
the pair of primers capable of hybridizing to the crtA gene of N. meningitidsis is SEQ ID NOs: 45 and 46, SEQ ID NOs: 47 and 48, SEQ ID NOs: 49 and 50, SEQ ID NOs: 51 and 52, or SEQ ID NOs: 53 and 54.

11. The method of claim 1, wherein the pair of control primers capable of hybridizing to the IAC is SEQ ID NOs: 60 and 61, SEQ ID NOs: 62 and 63, SEQ ID NOs: 64 and 65, SEQ ID NOs: 66 and 67, SEQ ID NOs: 68 and 69, SEQ ID NOs: 61 and 130, or SEQ ID NOs: 131 and 132.

12. The method of claim 1, wherein generating amplicons is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA).

13.-14. (canceled)

15. The method of claim 1, wherein each primer comprises exogenous nucleotide sequence.

16. The method of claim 1, wherein determining the presence or amount of one or more amplicons comprises contacting the amplicons with a plurality of oligonucleotide probes, wherein each of the plurality of oligonucleotide probes comprises a sequence selected from the group consisting of SEQ ID NOs: 11-15, 26-30, 40-44, 55-59, 70-74, and 133-134, or a sequence that exhibits at least about 85% identity to a sequence selected from the group consisting of SEQ ID NOs: 11-15, 26-30, 40-44, 55-59, 70-74, and 133-134.

17. The method of claim 16, wherein each of the plurality of oligonucleotide probes comprises a sequence selected from the group consisting of SEQ ID NOs: 11-15, 26-30, 40-44, 55-59, 70-74, and 133-134.

18. The method of claim 17, wherein each of the plurality of oligonucleotide probes consists of a sequence selected from the group consisting of SEQ ID NOs: 11-15, 26-30, 40-44, 55-59, 70-74, and 133-134.

19. The method of claim 16, wherein each probe is flanked by complementary sequences at the 5′ end and 3′ end.

20. The method of claim 19, wherein one of the complementary sequences comprises a fluorescence emitter moiety and the other complementary sequence comprises a fluorescence quencher moiety.

21. The method of claim 16, wherein at least one of the plurality of oligonucleotide probes comprises a fluorescence emitter moiety and a fluorescence quencher moiety.

22. A composition for the detection of S. pneumoniae and N. meningitidis in a sample, comprising:
at least one pair of primers capable of hybridizing to the lytA gene of S. pneumoniae, wherein each primer in said at least one pair of primers comprises any one of the sequences of SEQ ID NOs: 1-10, or a sequence that exhibits at least about 85% identity to any one of the sequences of SEQ ID NOs: 1-10;
at least one pair of primers capable of hybridizing to the cpsA gene of S. pneumoniae, wherein each primer in said at least one pair of primers comprises any one of the sequences of SEQ ID NOs: 16-25, or a sequence that exhibits at least about 85% identity to any one of the sequences of SEQ ID NOs: 16-25;
at least one pair of primers capable of hybridizing to the sodC gene of N. meningitidis, wherein each primer in said at least one pair of primers comprises any one of the sequences of SEQ ID NOs: 31-39, or a sequence that exhibits at least about 85% identity to any one of the sequences of SEQ ID NOs: 31-39; and
at least one pair of primers capable of hybridizing to the crtA gene of N. meningitidis, wherein each primer in said at least one pair of primers comprises any one of the sequences of SEQ ID NOs: 45-54, or sequence that exhibits at least about 85% identity to any one of the sequences of SEQ ID NOs: 45-54.

23. The composition of claim 22, further comprising at least one pair of control primers capable of hybridizing to an internal amplification control (IAC), wherein each primer in said at least one pair of control primers comprises any one of the sequences of SEQ ID NOs: 60-69 and 130-132, or a sequence that exhibits at least about 85% identity to any one of the sequences of SEQ ID NOs: 60-69 and 130-132.

24.-29. (canceled)

30. A method of detecting S. aureus, H. influenzae, M. catarrhalis, and K. pneumoniae in a sample, comprising:
contacting said sample with a plurality of pairs of primers, wherein the plurality of pairs of primers comprises:
at least one pair of primers capable of hybridizing to the nuc gene of S. aureus, wherein each primer in said at least one pair of primers comprises any one of the sequences of SEQ ID NOs: 75-83, or a sequence that exhibits at least about 85% identity to any one of the sequences of SEQ ID NOs: 75-83;
at least one pair of primers capable of hybridizing to the fucK gene of H. influenzae, wherein each primer in said at least one pair of primers comprises any one of the sequences of SEQ ID NOs: 88-95, or a sequence that exhibits at least about 85% identity to any one of the sequences of SEQ ID NOs: 88-95;
at least one pair of primers capable of hybridizing to the copB gene of M. catarrhalis, wherein each primer in said at least one pair of primers comprises any one of the sequences of SEQ ID NOs: 101-110, or a sequence that exhibits at least about 85% identity to any one of the sequences of SEQ ID NOs: 101-110; and
at least one pair of primers capable of hybridizing to the gltA gene of K. pneumoniae, wherein each primer in said at least one pair of primers comprises any one of the sequences of SEQ ID NOs: 116-124, or a sequence that exhibits at least about 85% identity to any one of the sequences of SEQ ID NOs: 116-124;
generating amplicons of the nuc gene sequence of S. aureus, amplicons of the fucK gene sequence of H. influenzae, amplicons of the gene sequence of the copB gene sequence of M. catarrhalis, amplicons of the gltA gene sequence of K. pneumoniae from said sample, or any combination thereof, if said sample comprises one or more of S. aureus, H. influenzae, M. catarrhalis, and K. pneumoniae; and
determining the presence or amount of one or more amplicons as an indication of the presence of one or more of S. aureus, H. influenzae, M. catarrhalis, and K. pneumoniae in said sample.

31.-95. (canceled)


SEQUENCE LISTING (139 sequences)

SEQ ID NO: 1 - DNA, 25 bp, Artificial Sequence
  Synthetic Oligonucleotide
cgtgagcagtttaagcatgatattg

SEQ ID NO: 2 - DNA, 24 bp, Artificial Sequence
  Synthetic Oligonucleotide
aagtagtaccaagtgccattgatt

SEQ ID NO: 3 - DNA, 22 bp, Artificial Sequence
  Synthetic Oligonucleotide
ggcattagccgtgagcagttta

SEQ ID NO: 4 - DNA, 23 bp, Artificial Sequence
  Synthetic Oligonucleotide
cgtaccagtagccagtgtcattc

SEQ ID NO: 5 - DNA, 21 bp, Artificial Sequence
  Synthetic Oligonucleotide
cggattatcactggcggaaag

SEQ ID NO: 6 - DNA, 23 bp, Artificial Sequence
  Synthetic Oligonucleotide
gcaccattatcaacaggtcctac

SEQ ID NO: 7 - DNA, 21 bp, Artificial Sequence
  Synthetic Oligonucleotide
aggctatatgcttgcagaccg

SEQ ID NO: 8 - DNA, 21 bp, Artificial Sequence
  Synthetic Oligonucleotide
gtagccatttcgcctgagttg

SEQ ID NO: 9 - DNA, 20 bp, Artificial Sequence
  Synthetic Oligonucleotide
ggctatatgcttgcagaccg

SEQ ID NO: 10 - DNA, 20 bp, Artificial Sequence
  Synthetic Oligonucleotide
tccagcctgtagccatttcg

SEQ ID NO: 11 - DNA, 30 bp, Artificial Sequence
  Synthetic Oligonucleotide
ctcaaacttgtcttttggataagagccgtc

SEQ ID NO: 12 - DNA, 33 bp, Artificial Sequence
  Synthetic Oligonucleotide
tctgccagcctgtttcaatcgtcaagccgttct

SEQ ID NO: 13 - DNA, 32 bp, Artificial Sequence
  Synthetic Oligonucleotide
catgatgcaaccgttcccaacaatgtgcgaga

SEQ ID NO: 14 - DNA, 33 bp, Artificial Sequence
  Synthetic Oligonucleotide
accagtaccagttgccgtctgtgtgcttcctcc

SEQ ID NO: 15 - DNA, 32 bp, Artificial Sequence
  Synthetic Oligonucleotide
ctgagttgtcgaaccagtaccagttgccgtct

SEQ ID NO: 16 - DNA, 22 bp, Artificial Sequence
  Synthetic Oligonucleotide
ggtgttctctatccttgtcagc

SEQ ID NO: 17 - DNA, 22 bp, Artificial Sequence
  Synthetic Oligonucleotide
agtcgcatttaaacgattggtc

SEQ ID NO: 18 - DNA, 20 bp, Artificial Sequence
  Synthetic Oligonucleotide
gcaccgactgggactgataa

SEQ ID NO: 19 - DNA, 21 bp, Artificial Sequence
  Synthetic Oligonucleotide
gctgccaagtaagacgaactc

SEQ ID NO: 20 - DNA, 23 bp, Artificial Sequence
  Synthetic Oligonucleotide
tggtgttctctatccttgtcagc

SEQ ID NO: 21 - DNA, 24 bp, Artificial Sequence
  Synthetic Oligonucleotide
cgcatttaaacgattggtcagtcc

SEQ ID NO: 22 - DNA, 22 bp, Artificial Sequence
  Synthetic Oligonucleotide
cccagtagggaatgtccatcta

SEQ ID NO: 23 - DNA, 21 bp, Artificial Sequence
  Synthetic Oligonucleotide
ggtcacggtctccatcggcta

SEQ ID NO: 24 - DNA, 23 bp, Artificial Sequence
  Synthetic Oligonucleotide
cctagtggtaactgcgttagtcc

SEQ ID NO: 25 - DNA, 24 bp, Artificial Sequence
  Synthetic Oligonucleotide
ccaacaaactgctgtactgcaaag

SEQ ID NO: 26 - DNA, 27 bp, Artificial Sequence
  Synthetic Oligonucleotide
ctgtgtcgctctttgcagtacagcagt

SEQ ID NO: 27 - DNA, 34 bp, Artificial Sequence
  Synthetic Oligonucleotide
tcgaccgtcaaatcggtattctgacttgacttaa

SEQ ID NO: 28 - DNA, 30 bp, Artificial Sequence
  Synthetic Oligonucleotide
acaaactgctgtactgcaaagagcgacaca

SEQ ID NO: 29 - DNA, 32 bp, Artificial Sequence
  Synthetic Oligonucleotide
agtagcgttcacgtacaaaacctagagcctgc

SEQ ID NO: 30 - DNA, 34 bp, Artificial Sequence
  Synthetic Oligonucleotide
cgacacagagctgacaaggatagagaacaccaac

SEQ ID NO: 31 - DNA, 23 bp, Artificial Sequence
  Synthetic Oligonucleotide
gtgcagtatgttcagttggtgtt

SEQ ID NO: 32 - DNA, 24 bp, Artificial Sequence
  Synthetic Oligonucleotide
ctggatcaagttgttgcactttca

SEQ ID NO: 33 - DNA, 23 bp, Artificial Sequence
  Synthetic Oligonucleotide
agcacacgagcataatacgatac

SEQ ID NO: 34 - DNA, 23 bp, Artificial Sequence
  Synthetic Oligonucleotide
gtacccacatctttgttaccgtt

SEQ ID NO: 35 - DNA, 22 bp, Artificial Sequence
  Synthetic Oligonucleotide
agtgcagtatgttcagttggtg

SEQ ID NO: 36 - DNA, 23 bp, Artificial Sequence
  Synthetic Oligonucleotide
ggtgatttacctgcattaactgt

SEQ ID NO: 37 - DNA, 23 bp, Artificial Sequence
  Synthetic Oligonucleotide
tgtggatcataatagagtgaccg

SEQ ID NO: 38 - DNA, 18 bp, Artificial Sequence
  Synthetic Oligonucleotide
gtgcagtatgttcagttg

SEQ ID NO: 39 - DNA, 24 bp, Artificial Sequence
  Synthetic Oligonucleotide
gaccatagttagattcagtaatag

SEQ ID NO: 40 - DNA, 29 bp, Artificial Sequence
  Synthetic Oligonucleotide
cgcaagcacacgagcataatacgatacct

SEQ ID NO: 41 - DNA, 30 bp, Artificial Sequence
  Synthetic Oligonucleotide
actggatcaagttgttgcactttcacttca

SEQ ID NO: 42 - DNA, 29 bp, Artificial Sequence
  Synthetic Oligonucleotide
tgcgcaagcacacgagcataatacgatac

SEQ ID NO: 43 - DNA, 27 bp, Artificial Sequence
  Synthetic Oligonucleotide
tgcatgatggcacagcaacaaatcctg

SEQ ID NO: 44 - DNA, 24 bp, Artificial Sequence
  Synthetic Oligonucleotide
cgcaagcacacgagcataatacga

SEQ ID NO: 45 - DNA, 19 bp, Artificial Sequence
  Synthetic Oligonucleotide
gaacgtcaggataaatgga

SEQ ID NO: 46 - DNA, 18 bp, Artificial Sequence
  Synthetic Oligonucleotide
gcatcagccatattcaca

SEQ ID NO: 47 - DNA, 20 bp, Artificial Sequence
  Synthetic Oligonucleotide
gccagaggcttatcgctttc

SEQ ID NO: 48 - DNA, 20 bp, Artificial Sequence
  Synthetic Oligonucleotide
tggcgtatagcggaacacaa

SEQ ID NO: 49 - DNA, 19 bp, Artificial Sequence
  Synthetic Oligonucleotide
cgcgtggtgtgtttgtgtt

SEQ ID NO: 50 - DNA, 20 bp, Artificial Sequence
  Synthetic Oligonucleotide
tgcctcactgccataacctt

SEQ ID NO: 51 - DNA, 21 bp, Artificial Sequence
  Synthetic Oligonucleotide
tgtgttccgctatacgccatt

SEQ ID NO: 52 - DNA, 20 bp, Artificial Sequence
  Synthetic Oligonucleotide
ctgcctcactgccataacct

SEQ ID NO: 53 - DNA, 20 bp, Artificial Sequence
  Synthetic Oligonucleotide
gcagtgaggcagagattcca

SEQ ID NO: 54 - DNA, 21 bp, Artificial Sequence
  Synthetic Oligonucleotide
ggcgagaacacaaacgacaaa

SEQ ID NO: 55 - DNA, 24 bp, Artificial Sequence
  Synthetic Oligonucleotide
taccgttggaatctctgcctcact

SEQ ID NO: 56 - DNA, 23 bp, Artificial Sequence
  Synthetic Oligonucleotide
cacaccacgcgcatcagaacggc

SEQ ID NO: 57 - DNA, 29 bp, Artificial Sequence
  Synthetic Oligonucleotide
tccgctatacgccattggtggaattgccg

SEQ ID NO: 58 - DNA, 29 bp, Artificial Sequence
  Synthetic Oligonucleotide
agcaatccatttatcctgacgttctgccg

SEQ ID NO: 59 - DNA, 27 bp, Artificial Sequence
  Synthetic Oligonucleotide
ttctgcacttcagccaacggcgcattc

SEQ ID NO: 60 - DNA, 19 bp, Artificial Sequence
  Synthetic Oligonucleotide
cagatcgatatgggtgccg

SEQ ID NO: 61 - DNA, 19 bp, Artificial Sequence
  Synthetic Oligonucleotide
cgagacgatgcagccattc

SEQ ID NO: 62 - DNA, 20 bp, Artificial Sequence
  Synthetic Oligonucleotide
cctaccgtctgctgtccaaa

SEQ ID NO: 63 - DNA, 20 bp, Artificial Sequence
  Synthetic Oligonucleotide
gtcgttgcgcggataaacaa

SEQ ID NO: 64 - DNA, 21 bp, Artificial Sequence
  Synthetic Oligonucleotide
agtgccacctaaattgtaagc

SEQ ID NO: 65 - DNA, 20 bp, Artificial Sequence
  Synthetic Oligonucleotide
actcaaccctatctcggtct

SEQ ID NO: 66 - DNA, 20 bp, Artificial Sequence
  Synthetic Oligonucleotide
ggatgaagttggtggtgagg

SEQ ID NO: 67 - DNA, 22 bp, Artificial Sequence
  Synthetic Oligonucleotide
gcctatcagaaacccaagagtc

SEQ ID NO: 68 - DNA, 21 bp, Artificial Sequence
  Synthetic Oligonucleotide
atggcaagaaagtgctcggtg

SEQ ID NO: 69 - DNA, 20 bp, Artificial Sequence
  Synthetic Oligonucleotide
gtgcagctcactcagtgtgg

SEQ ID NO: 70 - DNA, 26 bp, Artificial Sequence
  Synthetic Oligonucleotide
tctcatgcgtctccctggtgaatgtg

SEQ ID NO: 71 - DNA, 27 bp, Artificial Sequence
  Synthetic Oligonucleotide
tgccaaccatggcagccatgtgttaca

SEQ ID NO: 72 - DNA, 28 bp, Artificial Sequence
  Synthetic Oligonucleotide
ccaataggccgaaatcggcaaaatccct

SEQ ID NO: 73 - DNA, 26 bp, Artificial Sequence
  Synthetic Oligonucleotide
tctctgtctccacatgcccagtttct

SEQ ID NO: 74 - DNA, 28 bp, Artificial Sequence
  Synthetic Oligonucleotide
agtgatggcctggctcacctggacaacc

SEQ ID NO: 75 - DNA, 23 bp, Artificial Sequence
  Synthetic Oligonucleotide
caaccaatgacattcagactatt

SEQ ID NO: 76 - DNA, 23 bp, Artificial Sequence
  Synthetic Oligonucleotide
cgacttcaattttctttgcattt

SEQ ID NO: 77 - DNA, 18 bp, Artificial Sequence
  Synthetic Oligonucleotide
gtggttctgaagatccaa

SEQ ID NO: 78 - DNA, 19 bp, Artificial Sequence
  Synthetic Oligonucleotide
ggttgacctttgtacatta

SEQ ID NO: 79 - DNA, 24 bp, Artificial Sequence
  Synthetic Oligonucleotide
tgcgactttaattaaagcgattga

SEQ ID NO: 80 - DNA, 23 bp, Artificial Sequence
  Synthetic Oligonucleotide
actcgacttcaattttctttgca

SEQ ID NO: 81 - DNA, 21 bp, Artificial Sequence
  Synthetic Oligonucleotide
caaccaatgacattcagacta

SEQ ID NO: 82 - DNA, 21 bp, Artificial Sequence
  Synthetic Oligonucleotide
cttgcttcaggaccatatttc

SEQ ID NO: 83 - DNA, 18 bp, Artificial Sequence
  Synthetic Oligonucleotide
agtgcaacttcaactaaa

SEQ ID NO: 84 - DNA, 28 bp, Artificial Sequence
  Synthetic Oligonucleotide
tttcgtaaatgcacttgcttcaggacca

SEQ ID NO: 85 - DNA, 23 bp, Artificial Sequence
  Synthetic Oligonucleotide
aaccgtatcaccatcaatcgctt

SEQ ID NO: 86 - DNA, 27 bp, Artificial Sequence
  Synthetic Oligonucleotide
ttcgtaaatgcacttgcttcaggacca

SEQ ID NO: 87 - DNA, 27 bp, Artificial Sequence
  Synthetic Oligonucleotide
tggttgatacacctgaaacaaagcatc

SEQ ID NO: 88 - DNA, 23 bp, Artificial Sequence
  Synthetic Oligonucleotide
cgtcaatgctcactcaacgctta

SEQ ID NO: 89 - DNA, 23 bp, Artificial Sequence
  Synthetic Oligonucleotide
ctgcataacgcataggagggaaa

SEQ ID NO: 90 - DNA, 21 bp, Artificial Sequence
  Synthetic Oligonucleotide
gtttgctactcgtggagaaat

SEQ ID NO: 91 - DNA, 22 bp, Artificial Sequence
  Synthetic Oligonucleotide
tggaactgactgacttgctgta

SEQ ID NO: 92 - DNA, 24 bp, Artificial Sequence
  Synthetic Oligonucleotide
ctcactcaacgcttaactggtcaa

SEQ ID NO: 93 - DNA, 22 bp, Artificial Sequence
  Synthetic Oligonucleotide
tgctcactcaacgcttaactgg

SEQ ID NO: 94 - DNA, 23 bp, Artificial Sequence
  Synthetic Oligonucleotide
ccgtacctgtcatttcttgtgga

SEQ ID NO: 95 - DNA, 23 bp, Artificial Sequence
  Synthetic Oligonucleotide
cgtgctgggttctagccattaag

SEQ ID NO: 96 - DNA, 34 bp, Artificial Sequence
  Synthetic Oligonucleotide
tggtcaattcactacagatcacacaatggcggga

SEQ ID NO: 97 - DNA, 33 bp, Artificial Sequence
  Synthetic Oligonucleotide
atgtcagcgcattacaacatatggcaaatcaac

SEQ ID NO: 98 - DNA, 34 bp, Artificial Sequence
  Synthetic Oligonucleotide
cactacagatcacacaatggcgggaacatcaatg

SEQ ID NO: 99 - DNA, 35 bp, Artificial Sequence
  Synthetic Oligonucleotide
caattcactacagatcacacaatggcgggaacatc

SEQ ID NO: 100 - DNA, 33 bp, Artificial Sequence
  Synthetic Oligonucleotide
ccaggtgccagaacttaacacaggctgattcag

SEQ ID NO: 101 - DNA, 25 bp, Artificial Sequence
  Synthetic Oligonucleotide
gtcaggtagatgcccttgtctctta

SEQ ID NO: 102 - DNA, 21 bp, Artificial Sequence
  Synthetic Oligonucleotide
accacatcattgcccaacaga

SEQ ID NO: 103 - DNA, 20 bp, Artificial Sequence
  Synthetic Oligonucleotide
cattcgtggcgtgcgtgaag

SEQ ID NO: 104 - DNA, 24 bp, Artificial Sequence
  Synthetic Oligonucleotide
ctgaagctgttcgtcagtacgttt

SEQ ID NO: 105 - DNA, 20 bp, Artificial Sequence
  Synthetic Oligonucleotide
gcgtgcgtgaagagtttgac

SEQ ID NO: 106 - DNA, 21 bp, Artificial Sequence
  Synthetic Oligonucleotide
cccttgcctgcatatttgtta

SEQ ID NO: 107 - DNA, 23 bp, Artificial Sequence
  Synthetic Oligonucleotide
tgtggtcagccatctaaatgaag

SEQ ID NO: 108 - DNA, 19 bp, Artificial Sequence
  Synthetic Oligonucleotide
tagcgtcaaggcacgattg

SEQ ID NO: 109 - DNA, 20 bp, Artificial Sequence
  Synthetic Oligonucleotide
acaggtcgtacatggacagc

SEQ ID NO: 110 - DNA, 21 bp, Artificial Sequence
  Synthetic Oligonucleotide
ggcatctaccccttcaaccaa

SEQ ID NO: 111 - DNA, 33 bp, Artificial Sequence
  Synthetic Oligonucleotide
tggtgtaccctttaccgcctttatagtcgctgt

SEQ ID NO: 112 - DNA, 32 bp, Artificial Sequence
  Synthetic Oligonucleotide
tttgacttcgccaatcgtgccttgacgctaga

SEQ ID NO: 113 - DNA, 32 bp, Artificial Sequence
  Synthetic Oligonucleotide
tcgccaatcgtgccttgacgctagatatagaa

SEQ ID NO: 114 - DNA, 26 bp, Artificial Sequence
  Synthetic Oligonucleotide
agtcaaactcttcacgcacgccacga

SEQ ID NO: 115 - DNA, 28 bp, Artificial Sequence
  Synthetic Oligonucleotide
atgtcgcctatcgcctgccaaacccaag

SEQ ID NO: 116 - DNA, 20 bp, Artificial Sequence
  Synthetic Oligonucleotide
gcgatgaatctgccttcgtc

SEQ ID NO: 117 - DNA, 18 bp, Artificial Sequence
  Synthetic Oligonucleotide
gcctggtgcatctcgttc

SEQ ID NO: 118 - DNA, 18 bp, Artificial Sequence
  Synthetic Oligonucleotide
tcgagcaggcgatgaacc

SEQ ID NO: 119 - DNA, 18 bp, Artificial Sequence
  Synthetic Oligonucleotide
ggcgacgcaggcgaataa

SEQ ID NO: 120 - DNA, 22 bp, Artificial Sequence
  Synthetic Oligonucleotide
tgcgaatgctggaggagattga

SEQ ID NO: 121 - DNA, 19 bp, Artificial Sequence
  Synthetic Oligonucleotide
cccgatggctggtttcacg

SEQ ID NO: 122 - DNA, 17 bp, Artificial Sequence
  Synthetic Oligonucleotide
tgctgcaggtggcgatg

SEQ ID NO: 123 - DNA, 19 bp, Artificial Sequence
  Synthetic Oligonucleotide
cgacaggccattgtccaca

SEQ ID NO: 124 - DNA, 20 bp, Artificial Sequence
  Synthetic Oligonucleotide
tggtgcatctcgttccagtg

SEQ ID NO: 125 - DNA, 21 bp, Artificial Sequence
  Synthetic Oligonucleotide
agtgcgccacccacccgaccg

SEQ ID NO: 126 - DNA, 25 bp, Artificial Sequence
  Synthetic Oligonucleotide
atcctggtgctgcatgccgatcacg

SEQ ID NO: 127 - DNA, 26 bp, Artificial Sequence
  Synthetic Oligonucleotide
cgtcgatcaggttccggcgtttctgc

SEQ ID NO: 128 - DNA, 23 bp, Artificial Sequence
  Synthetic Oligonucleotide
cccttgaggacgtggcgctgacc

SEQ ID NO: 129 - DNA, 24 bp, Artificial Sequence
  Synthetic Oligonucleotide
ccacccacccgaccgtccgaccga

SEQ ID NO: 130 - DNA, 19 bp, Artificial Sequence
  Synthetic Oligonucleotide
cgagacgaagcagccattc

SEQ ID NO: 131 - DNA, 20 bp, Artificial Sequence
  Synthetic Oligonucleotide
gcttcctcgctcactgactc

SEQ ID NO: 132 - DNA, 23 bp, Artificial Sequence
  Synthetic Oligonucleotide
aaccgtattaccgcctttgagtg

SEQ ID NO: 133 - DNA, 26 bp, Artificial Sequence
  Synthetic Oligonucleotide
tgtcaagcgtctccctggtgaatgtg

SEQ ID NO: 134 - DNA, 26 bp, Artificial Sequence
  Synthetic Oligonucleotide
tgataccgctcgccgcagccgaacga

SEQ ID NO: 135 - DNA, 141 bp, Artificial Sequence
  Synthetic Oligonucleotide
cagatcgatatgggtgccgttcgagcagtttagccctaaatcaccctaccggcagacgtatgtcacattcaccagggagacgcttgacattggatgctgttgtgcgccctcaacaatgtaacgaatggctgcttcgtctcg

SEQ ID NO: 136 - DNA, 83 bp, Artificial Sequence
  Synthetic Oligonucleotide
gcttcctcgctcactgactcgctgcgctcggtcgttcggctgcggcgagcggtatcagctcactcaaaggcggtaatacggtt

SEQ ID NO: 137 - DNA, 141 bp, Artificial Sequence
  Synthetic Oligonucleotide
agtgccacctaaattgtaagcgttaatattttgttaaaattcgcgttaaatttttgttaaatcagctcattttttaaccaataggccgaaatcggcaaaatcccttataaatcaaaagaatagaccgagatagggttgagt

SEQ ID NO: 138 - DNA, 119 bp, Artificial Sequence
  Synthetic Oligonucleotide
ggatgaagttggtggtgaggccctgggcaggttggtatcaaggttacaagacaggtttaaggagaccaatagaaactgggcatgtggagacagagaagactcttgggtttctgataggc

SEQ ID NO: 139 - DNA, 89 bp, Artificial Sequence
  Synthetic Oligonucleotide
atggcaagaaagtgctcggtgcctttagtgatggcctggctcacctggacaacctcaagggcacctttgccacactgagtgagctgcac